(19) United States
(12) Patent Application Publication
(10) Pub. No.: US 2022/0170110 A1
(43) Pub. Date: Jun. 2, 2022

(54) CANCER DIAGNOSTIC MARKER USING TRANSPOSASE-ACCESSIBLE CHROMATIN SEQUENCING INFORMATION ABOUT INDIVIDUAL, AND USE THEREOF

(71) Applicant: KOREA ADVANCED INSTITUTE OF SCIENCE AND TECHNOLOGY, Daejeon (KR)

(72) Inventors: Daeyoup LEE, Daejeon (KR); Taemook KIM, Daejeon (KR); Sungwook HAN, Daejeon (KR)

(21) Appl. No.: 17/601,332

(22) PCT Filed: Nov. 19, 2019
(86) PCT No.: PCT/KR2019/015856

§ 371 Date: Oct. 4, 2021

(30) Foreign Application Priority Data
Apr. 5, 2019 (KR) 10-2019-0040056
Nov. 18, 2019 (KR) 10-2019-0147570

(51) Int. Cl. C12Q 1/6886

(52) U.S. Cl. CPC C12Q 1/6886; C12Q 2600/16

(57) ABSTRACT

The present invention relates to a cancer diagnostic marker screened using assay for transposase-accessible chromatin using sequencing (ATAC sequencing), and the use thereof. The open chromatin structural variation marker according to the present invention is useful as a cancer diagnostic marker because it can confirm the structural variation of chromatin with high accuracy. In addition, the open chromatin structural variation marker may be used as a new cancer diagnostic marker when detecting chromatin structural variation using a composition for detecting the marker.

### TECHNICAL FIELD

The present invention relates to a cancer diagnostic marker screened using assay for transposase-accessible chromatin using sequencing (ATAC sequencing), and the use thereof, and more particularly to an open chromatin structural variation marker obtained by treating a biological sample with transposase, extracting DNA therefrom, obtaining reads of the DNA, dividing the genome region into bins, and comparing the distribution of the number of reads in each bin with a reference population, and a method for diagnosing cancer using the same.

### BACKGROUND ART

Cancer deaths have increased not only in Korea but also worldwide. In Korea, there are patients with various cancers such as gastric cancer, breast cancer, thyroid cancer, lung cancer, and colorectal cancer. The causes of cancer are divided into congenital genetic mutations, and acquired factors, and cancer is not caused by mutation of a part of a specific gene, but is caused by a combination of various factors. Methods that are used to treat cancer include surgical transplantation and removal methods, chemotherapy and radiotherapy. Recently, the recurrence rate of cancer has been gradually decreasing through these methods, but studies have been steadily conducted to find the root cause of cancer and predict the prognosis thereof.

Next-generation sequencing (NGS) is a sequencing method that divides the genome into small segments and analyzes the genetic information of each segment in parallel. With the development of gene analysis technology, NGS has been used for genetic mutation detection, because it requires relatively short testing time and low cost and is capable of detecting even single nucleotide polymorphisms (SNPs) and insertions/deletions (INDELs) with high resolution. However, due to the principal nature of NGS that analyzes the genome divided into small segments, NGS has technical limitations in detecting large-scale structural variations or CNVs in the genome (Yoke S, Thyagarajan B. 2017, Arch Pathol Lab Med. Vol. 141(11), pp. 1544-1557).

To date, genome analysis and whole-genome analysis related to specific risk factors have been performed for research on specific genes related to various cancers. Although there are genetic risk factors for specific genes in relation to various cancers, most of these factors exist in the non-coding region, not the coding region, and it takes a lot of time to analyze these factors. For this reason, a new approach has been needed.

To solve this problem, epigenomic analysis techniques have been applied to interpret the function of genetic factors in the non-coding region. Histone modification studies using ChIP-Seq (Chromatin ImmunoPrecipitation Sequencing), one of the representative epigenomic analysis techniques, indicate the activity of the non-coding region of chromatin, and thus have been used as a method of elucidating the molecular mechanisms of cancer-causing genetic mutations through epigenomic mapping in cancer-related cell lines or tissues (Nevedomskaya et al., Genomics data vol. 2 195-8. 8 Jul. 2014).

However, this method is excessively dependent on an antibody used to precipitate a specific protein, and has difficulty in achieving more precise predictions because about 150 markers are used in epigenomic analysis. In addition, studies have reported that gene regulatory elements in the non-coding regions often regulate other distal genes rather than the nearest gene. Even though the gene regulatory elements and the distal genes are far apart from each other on the DNA due to the three-dimensional structure of chromatin, they can become close to each other in space through DNA folding. For this reason, it is difficult to clearly identify the root cause of cancer and the role of risk factors for prognosis prediction only by epigenomic mapping (Mishra et al., Genome medicine vol. 9, 1 87. 30 Sep. 2017).

Thus, in order to solve this problem, studies based on the three-dimensional structure of chromatin are needed to understand cancer-specific gene regulatory mechanisms, and a new study technique is needed for this purpose.

Techniques for studying the structure of chromatin include ATAC-Seq (Assay for Transposase-Accessible Chromatin using Sequencing) and Hi-C using NGS. Hi-C is a representative technique of studying the structure of chromatin at high resolution based on 3C (Chromosome Conformation Capture), and is a technique of capturing the physical association of chromatin in the genome (Belton et al., Methods (San Diego, Calif.) vol. 58, 3 (2012)). ATAC-Seq is a technique of detecting open regions of chromatin using transposons, and has advantages in that it may be sufficiently performed even with a small amount of a sample, may be used for rare cell lines or patients, and is cost-effective compared to Hi-C (Buenrostro et al., Nature methods vol. 10, 12, 2013).

Accordingly, the present inventors have made extensive efforts to develop an open chromatin structural variation marker based on ATAC-Seq, and as a result, have found that cancer can be diagnosed with high accuracy by dividing the genome into highly enriched bins using ATAC-Seq results, selecting marker candidates through comparison of the number of reads with that in a reference population, selecting a marker that is statistically significant compared to the reference population, and analyzing the structure of chromatin in the marker. Based on this finding, the present invention has been completed.

The above information disclosed in this Background section is only for enhancement of understanding of the background of the present invention. Therefore, it may not contain information that forms the conventional art that is already known in the art to which the present invention pertains.

### SUMMARY OF THE INVENTION

An object of the present invention is to provide a composition for diagnosing breast cancer, which is capable of detecting a chromatin structural variation marker.

Another object of the present invention is to provide a method of diagnosing breast cancer using the composition for diagnosing breast cancer.

To achieve the above objects, the present invention provides a composition for diagnosing breast cancer containing: transposase; and a primer pair specific to any one nucleic acid selected from the group consisting of SEQ ID NOs: 1 to 100.

The present invention also provides a method for diagnosing breast cancer comprising steps of: obtaining a nucleic acid fragment by treating a nucleic acid, isolated from a biological sample, with transposase; and detecting the chromatin structure of the nucleic acid by amplifying the obtained nucleic acid fragment using primer pairs specific to any one or more nucleic acids selected from the group consisting of SEQ ID NOs: 1 to 100.

### BRIEF DESCRIPTION OF DRAWINGS

FIG. 1 is an overall flowchart showing a method for screening an open chromatin structural variation marker according to the present invention.

FIG. 2 is a graph showing the distribution of chromatin structure variation candidate markers for normal and triple-negative breast cancer samples, detected according to one example of the present invention.

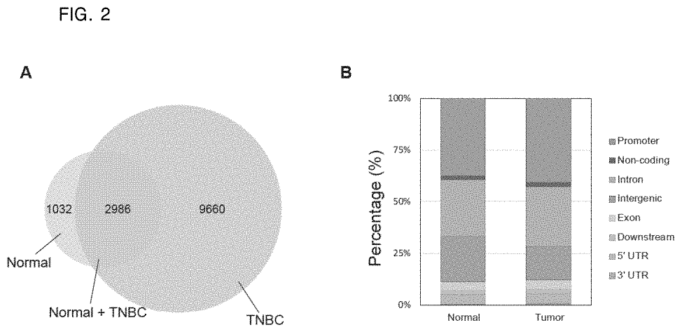

FIG. 3 is a graph showing a flowchart for detecting a region having a large structural difference between normal and triple negative breast cancer samples, among triple-negative breast cancer-specific genetic structural variation markers detected according to one example of the present invention.

FIG. 4 is a graph showing differences in structural variation markers between normal and triple-negative breast cancer samples, determined using a heat map according to one example of the present invention.

FIG. 5 is a genome-wide graph showing examples of triple-negative breast cancer-specific genetic structural variation markers detected according to one example of the present invention.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

In the present invention, it was attempted to determine whether cancer could be diagnosed using an open chromatin structural variation marker screened using ATAC-seq.

In the present invention, it has been found that, when an open chromatin structural mutation marker is screened by ATAC-seq through comparison with a normal reference population and the possibility of cancer in a sample is detected using the marker, cancer can be diagnosed using the open chromatin structural mutation marker with high accuracy.

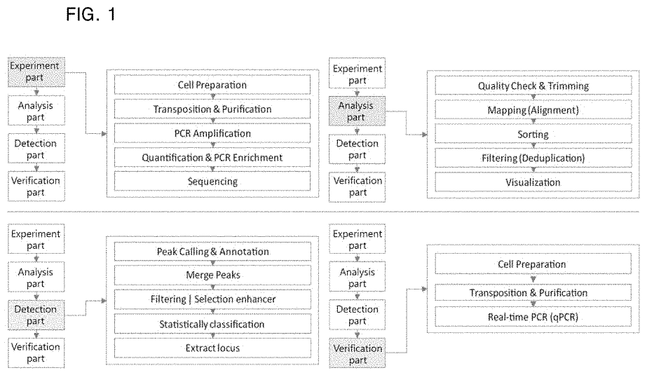

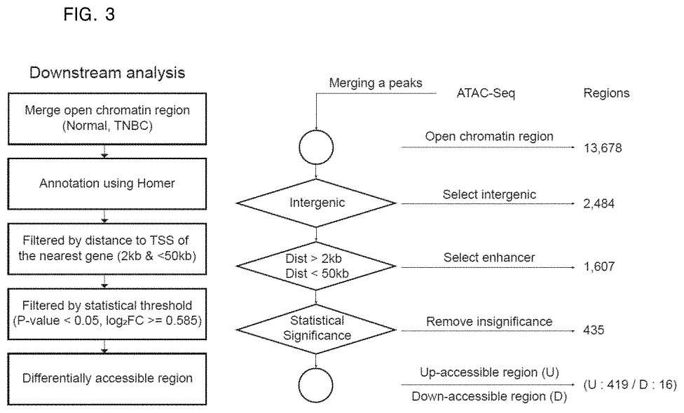

That is, in one example of the present invention, DNA was extracted from transposase-treated cells and subjected to NGS. Then, the sequence was aligned based on the reference genome Hg19 sequence, and the quality thereof was evaluated. Then, the genome was divided into highly enriched bins, and the number of matched reads for each bin was graphically expressed. Then, a bin having a value equal to or higher than a reference value was selected, and the selected bin was selected as an open chromatin structural variation marker when the read peak value thereof was different from that of a reference population. Another sample was treated with transposase, and then the selected marker was detected by real-time PCR using primers capable of amplifying the marker. As a result, it was confirmed that cancer diagnosis could be performed with high accuracy based on the three-dimensional structure of chromatin (FIGS. 1 and 3).

Therefore, in one aspect, the present invention is directed to a composition for diagnosing breast cancer containing: transposase and

a primer pair specific to any one nucleic acid selected from the group consisting of SEQ ID NOs: 1 to 100.

In the present invention, the primer pair that binds specifically to each of the nucleic acids may be a primer pair that binds specifically to each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleic acids selected from the group consisting of SEQ ID NOs: 1 to 100. Preferably, the primer pair may comprise a primer pair specific to each of the nucleic acids represented by the sequences of SEQ ID NOs: 1 to 20.

In the present invention, the primer pair may comprise a primer pair specific to each of the nucleic acids represented by the sequences of SEQ ID NOs: 41 to 60.

In the present invention, the primer pair may comprise a primer pair specific to each of the nucleic acids represented by the sequences of SEQ ID NOs: 61 to 80.

In the present invention, the primer pair may comprise a primer pair specific to each of the nucleic acids represented by the sequences of SEQ ID NOs: 81 to 100.

In the present invention, the term “breast cancer” refers to cancer occurring in the breast, and may be used interchangeably with “mammary gland cancer”. The breast cancer may include mammary gland breast cancer, lobule breast cancer, or a combination thereof. According to the site of occurrence, breast cancer may be broadly classified into two types: cancer occurring in the ductal and lobular epithelium, and cancer occurring in the stroma. The breast cancer may include a type of complex carcinoma (CC) or ductal carcinoma (DC). The ductal carcinoma is a type of breast cancer that exists primarily in the ducts of an individual.

In the present invention, the term “diagnosis” refers to diagnosing a disease, and may include the name, state, stage, etiology, presence or absence of complications, prognosis, and recurrence of breast cancer.

In the present invention, the term “transposase” refers to an enzyme that binds to the end of a transposon and catalyzes the movement of the transposon to another part of the genome by cut and paste or replicative transposition. The transposase may be an enzyme classified as EC number EC 2.7.

In the present invention, the transposase may be Tn5 transposase. The Tn5 transposase is a member of the RNase superfamily including retroviral integrases. Tn5 transposase catalyzes “cut and paste” transposition. Tn5 transposase may be used in a genome sequencing method using DNA fragmentation, the so-called ATAC-seq technique.

In the present invention, the term “amplification” refers to a reaction for amplifying a nucleic acid molecule. A number of amplification reactions have been reported in the art, including, but not limited to, polymerase chain reaction (hereinafter referred to as PCR) (U.S. Pat. Nos. 4,683,195, 4,683,202, and 4,800,159), reverse transcription-polymerase chain reaction (hereinafter referred to as RT-PCR) (Sambrook, J. et al., Molecular Cloning. A Laboratory Manual, 3rd ed. Cold Spring Harbor Press (2001)), the methods of WO 89/06700 and EP 329,822, ligase chain reaction (LCR), repair chain reaction (EP 439,182), transcription-mediated amplification (TMA; WO 88/10315), self-sustained sequence replication (WO 90/06995), selective amplification of target polynucleotide sequences (U.S. Pat. No. 6,410,276), consensus sequence primed polymerase chain reaction (CP-PCR; U.S. Pat. No. 4,437,975), arbitrarily primed polymerase chain reaction (AP-PCR; U.S. Pat. Nos. 5,413,909 and 5,861,245), nucleic acid sequence based amplification (NASBA; U.S. Pat. Nos. 5,130,238, 5,409,818, 5,554,517 and 6,063,603), strand displacement amplification, and loop-mediated isothermal amplification (LAMP).

Other amplification methods that may be used are described in U.S. Pat. Nos. 5,242,794, 5,494,810, 4,988,617 and in U.S. Ser. No. 09/854,317.

PCR is one of the most predominant processes for nucleic acid amplification, and many variations and applications thereof have been developed. For example, touchdown PCR, hot start PCR, nested PCR and booster PCR have been developed by modifying traditional PCR procedures to improve PCR specificity or sensitivity. In addition, real-time PCR, differential display PCR (DD-PCR), rapid amplification of cDNA ends (RACE), multiplex PCR, inverse polymerase chain reaction (IPCR), vectorette PCR and thermal asymmetric interlaced PCR (TAIL-PCR) have been developed for certain applications. Details on PCR are described in McPherson, M. J., and Moller, S. G. PCR. BIOS Scientific Publishers, Springer-Verlag New York Berlin Heidelberg, N.Y. (2000), the teachings of which are incorporated herein by reference.

In the present invention, multiplex amplification is multiplex PCR (polymerase chain reaction) amplification. According to one embodiment of the present invention, the multiplex PCR amplification has an annealing temperature condition of 57 to 61° C. According to another embodiment of the present invention, the multiplex PCR amplification has an annealing temperature condition of 58 to 60° C. According to a specific embodiment of the present invention, the multiplex PCR amplification has an annealing temperature condition of 58.5 to 59.5° C.

The multiplex PCR amplification requires an appropriate number of cycles to perform PCR. According to one embodiment of the present invention, the multiplex PCR amplification is performed for 27 to 30 cycles. When the multiplex PCR amplification of the present invention was performed for 26 cycles or less, peaks of 500 RFU or less were formed, and when the multiplex PCR amplification was performed for 31 cycles, a peak of 2,000 RFU or more was formed, but noise increased and incomplete A insertion undesirably occurred.

In the present invention, the composition may contain at least one adaptor. The adaptor refers to a short, synthesized oligonucleotide which is used in genetic engineering. The transferase may be a transposase complex having one or two adaptors conjugated thereto. The adapter may be inserted into either or both ends of the nucleic acid fragment by cut and paste of the transposase. The adapter may comprise a sequence identical to or complementary to a primer for nucleic acid amplification.

In the present invention, the nucleic acid comprises genomic DNA, chromatin, and fragments thereof. The nucleic acid may comprise an open reading frame (ORF) and control regions. The control regions include a promoter, an enhancer, a silencer, and an untranslated region (UTR).

In the present invention, the term “primer” refers to a single-stranded oligonucleotide that may act as the starting point of template-directed DNA synthesis under suitable conditions (that is, four different nucleoside triphosphates and polymerase) in a suitable buffer solution at a suitable temperature. The suitable length of the primer may vary depending on various factors, for example, a temperature and the intended use of the primer, but is typically 15 to 30 nucleotides. A short primer may generally require a lower temperature to form a sufficiently stable hybrid complex with a template. The terms “forward primer” and “reverse primer” refer to primers that bind to the 3′ and 5′ ends, respectively, of a specific region of a template which is amplified by polymerase chain reaction. The sequence of the primer does not need to have a sequence perfectly complementary to a partial sequence of the template, and is sufficient if it has sufficient complementarity within the range within which it may hybridize with the template and is capable of performing the intrinsic action of the primer. Thus, it is believed that the primer set according to one embodiment does not need to have a sequence perfectly complementary to the template nucleotide sequence and is sufficient if it has sufficient complementarity within the range in which it may hybridize with this sequence and act as a primer. The design of this primer may be easily performed by those skilled in the art with reference to the nucleotide sequence of the polynucleotide as a template, and may be performed, for example, using a primer design program (e.g., PRIMER 3, VectorNTI program).

In the present invention, the primer pair may be used without limitation as long as it is a primer pair capable of amplifying any one marker selected from among SEQ ID NOs: 1 to 100. Preferably, the primer pair may be any one primer pair selected from the group consisting of SEQ ID NOs: 101 to 300.

For example, the forward primer for amplifying the BC3M_102 marker sequence represented by SEQ ID NO: 1 according to the present invention is represented by SEQ ID NO: 101, and the reverse primer is represented by SEQ ID NO: 102. The forward primer for amplifying the BC3M_11 marker sequence represented by SEQ ID NO: 2 according to the present invention may represented by SEQ ID NO: 103, and the reverse primer may be represented by SEQ ID NO: 104.

In the present invention, the primer pair may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 primer pairs, among the primer pairs represented by SEQ ID NOs: 101 to 300. Preferably, the primer pair may be the primer pairs represented by SEQ ID NOs: 101 to 140.

In the present invention, the primer pair may be the primer pairs represented by SEQ ID NOs: 141 to 180.

In the present invention, the primer pair may be the primer pairs represented by SEQ ID NOs: 181 to 220.

In the present invention, the primer pair may be the primer pairs represented by SEQ ID NOs: 221 to 260.

In the present invention, the primer pair may be the primer pairs represented by SEQ ID NOs: 261 to 300.

All the marker sequences that are used in the present invention are shown in Table 2 below, and all the primer sequences that are used in the present invention are shown in Table 3 below.

In the present invention, the marker sequence may be screened by a method comprising steps of:

(a) treating a nucleic acid, isolated from a biological sample, with transposase, and obtaining DNA reads;

(b) aligning the reads to a reference genome database of a reference population;

(c) calculating sequencing quality scores for the aligned reads and selecting reads;

(d) dividing the open bin of the reference genome into highly enriched bins, calculating the number of reads in each bin for the selected reads, and excluding bins having an RPKM value of less than 5 as calculated by the following equation 1:

$$RPKM of a region = \frac{Number of read mapped to a region \times {10}^{3} \times {10}^{6}}{Total number of mapped reads \times Region length} \qquad [ Equation 1 ]$$

(e) performing comparison with the quantified value of the reference population, and selecting bins, which have a statistically significant difference, as open chromatin structural variation markers; and

(f) analyzing the selected markers by real-time PCR, and selecting a candidate, which shows an open chromatin structure different from the reference population, as an open chromatin structural variation marker.

In the present invention, the term “reads” refers to a single nucleic acid fragment obtained by analyzing sequence information using various methods known in the art. Thus, in the present specification, the terms “sequence information” and “reads” have the same meaning in that they are sequence information obtained through a sequencing process.

In the present invention, the term “bin” is used in the same sense as a specific region or a region, and refers to a part of the entire genome sequence.

In the present invention, the term “reference population” refers to a reference group that may be used for comparison, such as a reference nucleotide sequence database, and refers to a population of people who do not currently have a specific disease or condition. In the present invention, the reference nucleotide sequence in the reference genome sequence database of the reference population may be a reference genome generated using normal tissues of breast cancer patients, provided by Seoul National University Hospital.

In the present invention, the term “RPKM” is an abbreviation for reads per kilobase of transcript per million mapped reads, and refers to a normalized peak value.

This means the normalized peak value for open chromatin region. It is a value obtained by quantifying an open chromatin region based on the total number of mapped reads of the entire genome for reads mapped to the open chromatin region.

In the present invention, the chromatin includes euchromatin and heterochromatin. The chromatin may include nucleosomes, each composed of about two turns of DNA wrapped around eight histone protein cores. DNA regions between nucleosomes may have an “open chromatin” structure. Transcription factors, polymerases, etc. may attach to open chromatin to initiate transcription. The DNA region wrapped around histone protein cores may have a “closed chromatin” structure. Closed chromatin may bind DNA and histone proteins, and thus transcription factors and polymerases may not attach thereto. The structure of the chromatin may be changed depending on intracellular signaling and the like.

In the present invention, step (a) may be performed by a method comprising steps of:
(a-i) obtaining a cellular nucleus from a biological sample;
(a-ii) adding a transposase complex comprising a transposase and an adaptor to the obtained cellular nucleus to produce a nucleic acid fragment labeled with the adaptor at either or both ends;
(a-iii) obtaining a purified nucleic acid by removing protein, fat and other residues from the produced nucleic acid fragment using a salting-out method, a column chromatography method or a beads method;
(a-iv) constructing a single-end sequencing or pair-end sequencing library for the purified nucleic acid;
(a-v) reacting the constructed library with a next-generation sequencer; and
(a-vi) obtaining reads of the nucleic acid from the next-generation sequencer.

In the present invention, step (a) may be performed by the method further comprising, between steps (a-iii) and (a-iv), a step of constructing a single-end sequencing or pair-end sequencing library by randomly fragmenting the nucleic acid, in step (a-ii), by an enzymatic cleavage, atomization or Hydroshear method.

In the present invention, the next-generation sequencer may be, but is not limited, Illumina Company's Hiseq system, Illumina Company's Miseq system, Illumina Company's genomic analyzer (GA) system, Roche Company's 454 FLX from, Applied Biosystems Company's SOLiD system, or Life Technologies Company's Ion Torrent system.

In the present invention, the aligning step may be performed using, but not limited to, the BWA algorithm and the Hg19 sequence.

In the present invention, the BWA algorithm may include, but is not limited to, BWA-mem, BWA-ALN, BWA-SW or Bowtie2.

In the present invention, the term “selection of reads” in step (c) means a procedure of determining whether additional analysis based on the corresponding data is performed or ended, by checking whether quality scores, for example, sequencing quality scores, satisfy a certain requirement.

In the present invention, step (c) may comprise steps of:

(c-i) specifying the region of each aligned nucleic acid sequence; and

(c-ii) selecting a region having a sequencing quality score of 30 or more and exceeding 80% of the entire nucleic acid sequence region.

In the present invention, step (c) may further comprise a step of selecting a sequence, which satisfies a reference value of a mapping quality score, from the selected region.

In the present invention, in step (c-i) of specifying the region of the nucleic acid sequence, the region of the nucleic acid sequence may be, but is not limited to, 1 kb to 1 MB.

In the present invention, in step (c-ii), the sequencing quality score within the region may vary depending on a desired criterion, but is specifically 30 or more, and this step is a step of selecting a region having a sequencing quality score of 30 or more and exceeding 70%, more specifically 75%, most preferably 80% of the entire nucleic acid sequence region.

In the present invention, in step (c-iii), the reference value of the mapping quality score may vary depending on a desired criterion, but is specifically 15 to 70, more specifically 30 to 65, most preferably 60.

In the present invention, the highly enriched bin in step (d) may be 15 kb to 50 kb. That is, in the present invention, the bin may be, but is not limited to, kb to 1 MB, specifically 1 kb to 500 kb, more specifically 15 kb to 100 kb, even more specifically 15 kb to 50 kb, most preferably 15 kb.

In the present invention, the statistically significant difference in step (e) may be a p-value of less than 0.05 as calculated by the following equation 2, and may be a fold change of 1.5 or more as calculated by the following equation 3:

$$t = \frac{\overline{{X}_{1}} - \overline{{X}_{2}}}{{s}_{( \overline{{X}_{1}} - \overline{{X}_{2}} )}} \\ Where \\ {s}_{( \overline{{X}_{1}} - \overline{{X}_{2}} )} = \sqrt{\frac{{s}_{1}^{2}}{{n}_{1}} + \frac{{s}_{2}^{2}}{{n}_{2}}} \qquad [ Equation 2 ]$$

wherein X1 and X2 represent RPKM average values for groups (1: control group, and 2: comparison group), and n1 and n2 represent the number of samples corresponding to each group.

For example, when two groups (Normal and Cancer) are compared, if there are 10 normal samples and 10 cancer samples, X1 means the average value for 10 normal samples, and X2 means the average value for 10 cancer samples.

$${Log}_{2} Fold Change ( 2 FC ) = {Log}_{2} ( \frac{Treatment}{Control} ) \qquad [ Equation 3 ]$$

wherein control means a control group, and treatment means a comparison group.

In the present invention, the control group is preferably a normal cell group or a cell group having a disease other than a target disease, and the comparison group may be a target disease cell group, preferably a specific cancer cell group.

In the present invention, step (f) may comprise steps of:

(f-i) obtaining a nucleic acid fragment by treating a nucleic acid, isolated from a biological sample, with transposase; and

(f-ii) detecting the chromatin structure of the nucleic acid by amplifying the nucleic acid fragment using primers capable of amplifying the nucleic acid fragment.

The term “reference genome” in the present invention is a combination of genetic information from multiple donors determined to be genetically normal, and may be, for example, GRCh37(Hg19) data provided by NCBI.

In another aspect, the present invention is directed to a method for diagnosing breast cancer comprising steps of:

obtaining a nucleic acid fragment by treating a nucleic acid, isolated from a biological sample, with transposase; and

detecting the chromatin structure of the nucleic acid by amplifying the obtained nucleic acid using primer pairs specific to any one or more nucleic acids selected from the group consisting of SEQ ID NOs: 1 to 100.

In the present invention, the primer pairs may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 primer pairs, among the primer pairs represented by SEQ ID NOs: 101 to 300. Preferably, the primer pairs may be the primer pairs represented by SEQ ID NOs: 101 to 140.

In the present invention, the primer pairs may further comprise the primer pairs represented by SEQ ID NOs: 141 to 180.

In the present invention, the primer pairs may further comprise the primer pairs represented by SEQ ID NOs: 181 to 220.

In the present invention, the primer pairs may further comprise the primer pairs represented by SEQ ID NOs: 221 to 260.

In the present invention, the primer pairs may further comprise the primer pairs represented by SEQ ID NOs: 261 to 300.

In the present invention, the biological sample may be blood, bone marrow aspirate, lymphatic fluid, saliva, lacrima, mucosal fluid, amniotic fluid, or cells isolated therefrom. The biological sample may be cells isolated from blood. For example, the cells are peripheral blood mononuclear cells (PBMCs).

In the present invention, the method of obtaining a cellular nucleus from the biological sample may be performed using a method commonly used in the art. For example, the nucleus may be isolated using a cell membrane degradation solution.

In the present invention, the method comprises a step of producing a nucleic acid fragment by adding transposase to the obtained cellular nucleus.

The transposase may bind to open chromatin. The transposase may bind non-specifically to open chromatin, so that it may cut the open chromatin between nucleosomes in the cellular nucleus.

The method comprises a step of detecting the chromatin structure of the nucleic acid by amplifying the nucleic acid fragment in the presence of a primer set specific to any one nucleic acid selected from the group consisting of SEQ ID NOs: 1 to 100.

When any one nucleic acid selected from the group consisting of SEQ ID NOs: 1 to 100 has an open chromatin structure, the nucleic acid nucleic acid may be produced by binding of transposase to the chromatin. When the produced nucleic acid fragment is amplified using a primer set specific to any one nucleic acid selected from the group consisting of SEQ ID NOs: 1 to 100, an amplification product may be produced from the nucleic acid. When any one nucleic acid selected from the group consisting of 1 to 100 has a closed chromatin structure, the transposase cannot bind to the nucleic acid and the nucleic acid fragment cannot be produced. When the reaction product is amplified using a primer pair specific to any one nucleic acid selected from the group consisting of SEQ ID NOs: 1 to 100, an amplification product may not be produced or may be less produced, because the nucleic acid fragment is not present.

That is, when the amount of amplification of any one nucleic acid selected from the group consisting of SEQ ID NOs: 1 to 100 is statistically significantly larger than that of the reference population, this means that the subject from whom the biological sample was isolated has a high probability of developing breast cancer.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### EXAMPLES

Hereinafter, the present invention will be described in more detail with reference to examples. It will be obvious to those skilled in the art that these examples serve only to illustrate the present invention, and the scope of the present invention is not limited by these examples.

### Example 1: Construction and Sequencing of ATAC Library for Each Carcinoma

About 20 mg of frozen tissue was disrupted, and nuclei were isolated therefrom using nuclei isolation buffer (NIB), and then a large tissue mass was removed therefrom by filtration. Tagmentation was performed using TD buffer and Tn5 transposase (Addgene, pTXB1-Tn5 vector). Thereafter, Nextera PCR primers were attached using a HiFi Hotstart ReadyMix (KAPA: KK2601) kit, and then PCR amplification was performed. An ATAC library was constructed using the PCR amplified DNA, and then purified using a Qiagen PCR purification kit. Sequences were read using a next-generation sequencer which is an Illumina Hiseq4000 system.

TABLE 1
Primer sequences

| SEQ | | |
|---|---|---|
| ID | Tagmentation | |
| NO | index primer | Sequence |
| | Ad1_noMX | |
| 301 | Ad2.1 | TAAGGCGA |
| 302 | Ad2.2 | CGTACTAG |
| 303 | Ad2.3 | AGGCAGAA |
| 304 | Ad2.4 | TCCTGAGC |
| 305 | Ad2.5 | GGACTCCT |
| 306 | Ad2.6 | TAGGCATG |
| 307 | Ad2.7 | CTCTCTAC |
| 308 | Ad2.8 | CAGAGAGG |
| 309 | Ad2.9 | GCTACGCT |
| 310 | Ad2.10 | CGAGGCTG |
| 311 | Ad2.11 | AAGAGGCA |
| 312 | Ad2.12 | GTAGAGGA |
| 313 | Ad2.13 | GTCGTGAT |
| 314 | Ad2.14 | ACCACTGT |
| 315 | Ad2.15 | TGGATCTG |
| 316 | Ad2.16 | CCGTTTGT |
| 317 | Ad2.17 | TGCTGGGT |
| 318 | Ad2.18 | GAGGGGTT |
| 319 | Ad2.19 | AGGTTGGG |
| 320 | Ad2.20 | GTGTGGTG |
| 321 | Ad2.21 | TGGGTTTC |
| 322 | Ad2.22 | TGGTCACA |
| 323 | Ad2.23 | TTGACCCT |
| 324 | Ad2.24 | CCACTCCT |

### Example 2: Pre-Processing Analysis

Before open chromatin regions were found using reads, sequence quality checking was performed using FastQC, a representative sequence checking program, in order to confirm whether the DNA sequences were accurately read using Illumina Hiseq4000. When adaptors and primers were read in some sequences or when the quality of the sequences was low, the misread sequences and low-quality sequences (Q20 or less) were removed using a removal program such as Trim galore or Trimmomatic.

In order to check where the short sequences that have been quality-checked originated from the already known human reference genome sequence, a mapping (alignment) process was performed using Bowtie2, a representative mapping program.

Thereafter, for downstream analysis, sorting and indexing were performed using the Samtools program. Since biased data generated during the experimental process (PCR) were present in the mapped sequences, the duplicated sequences generated during PCR were removed using Picard (MarkDuplicates) in order to remove the biased data.

### Example 3: Peak Calling and Classification

To detect open chromatin regions for each carcinoma, Genrich tool was used to detect open chromatin regions. More accurate information about each open chromatin region was described through annotation of the open chromatin regions extracted as described above.

To confirm the change in the chromatin structure of the enhancer region, the peaks present in the intergenic region were extracted, and thereamong, targets located at more than 2 kb and less than 50 kb from the transcription start site (TSS) were used. Homer (MergePeak) was used to classify specific and common chromatin structural changes for normal and breast cancer tissues. To solve the problem of recognizing some bias as a peak, an operation of removing peaks that do not exceed a reference value (threshold value: RPKM <5, equation 1) was performed, followed by a process of reclassifying the parts where a statistically significant difference between the two groups (p-value <0.05 Equation 2; fold change: 1.5 times or more, equation 3) occurred.

$$\begin{matrix}RPKM of a region = \frac{Number of read mapped to a region \times {10}^{3} \times {10}^{6}}{Total number of mapped reads \times Region length} & [ Equation 1 ] \\ t = \frac{\overline{{X}_{1}} - \overline{{X}_{2}}}{{s}_{( \overline{{X}_{1}} - \overline{{X}_{2}} )}} \\ Where \\ {s}_{( \overline{{X}_{1}} - \overline{{X}_{2}} )} = \sqrt{\frac{{s}_{1}^{2}}{{n}_{1}} + \frac{{s}_{2}^{2}}{{n}_{2}}} & [ Equation 2 ]\end{matrix}$$

wherein X1 and X2 represent RPKM average values for groups (1: control group, and 2: comparison group), and n1 and n2 represent the number of samples corresponding to each group.

$${Log}_{2} Fold Change ( 2 FC ) = {Log}_{2} ( \frac{Treatment}{Control} ) \qquad [ Equation 3 ]$$

wherein control means a control group, and treatment means a comparison group.

As a result, open chromatin structural variation markers specific for breast cancer were identified (FIGS. 3, 4 and 5).

TABLE 2
Open chromatin structural variation markers specific to breast cancer

| SEQ | | |
|---|---|---|
| ID | | |
| NO | Name | Sequence |
| 1 | BC3M_102 | GGGATCCCTCAGCAGCTCCGGACCTCATCTGCCCCACTTCGGCATCCCGCGCGGGA |
| | | ATATGACCATGTAGGAGTAACCCGGGGCTCTCAAGGACTCTACGGTTTGTCACGGT |
| | | TTGAACGCAAGCGCAGGGCCTGGGGCGGGTGCAGGTGGAGGGTCGGCCTCTTTCTG |
| | | CCCTTGGGAACGCCCCTTTCTGGATGTGGACCGGCGAGGCGGTCTCTCCTTTCTGC |
| | | CCTCGCCTGGTGAAATGTGGGCACTGCTGCCAGGAGAAAAAAAACTGAAGCTGTGA |
| | | ATTCAGTTCATCACCCTTCC |
| 2 | BC3M_11 | AGCGGGGCTAGACGGAGTCAGGGGCGGACCGCCACAGCCTGCACCAATCAGGACCC |
| | | GGTTGATAGGCAGAGCCTGGCGACTTCGAAGACTCGCCCCCAGTCAAAGAGCCCCG |
| | | GGGATTCGTTTCCGTACGCAGCCTGGAAACCAGCCTGGGCCTATCCTGCGCGCCGC |
| | | TGCGGGCTACTATTGGCTGCCAAGAAACCCCGCCCATCTTCCTGCTCATTGGCCGG |
| | | TGCGGTTTACGTAAGAGGAGCCTGTTGCTGAGCGAAAAGTCTGTTCTGCAATTTTC |
| | | GCTAAGGAGTTGTTAACGCT |
| 3 | BC3M_117 | GTACACTGACTTTGGAACAAATGCCACAGGCCCTAATTGCAGGCTCCAAGGAGTTG |
| | | AGATTCCATACTGGGGTTGCTGGAGGCAGAAGCCTTCCCACTTTCAGGACCCGGAC |
| | | CTGCCCTTCCCCCACGCGGTCCCGCCCAGCCAGCTACACCCTGGCCACAGAGCGCT |
| | | CACAAAGGCTCAGTGTGTGTATGCCGGGCTGACTCACAGTGGTTCTGGGCCCAGGC |
| | | GAGGACCTTCTCAGAGGGGCGGAAGGGGCCCTCTCCCTCCTGGCCATTTTCCATGG |
| | | GGAGCAGTCAGTAACCAGGA |
| 4 | BC3M_119 | CTAGGAGACAAGTACCCTGCTGAGCAGACAAATAGCCTGGACTTTGTAACAGCCAA |
| | | AGTGGCCCACATGGCACTCGCGGGGCTGTGCAGCATCCAGGCAGGGGACACTGCCT |
| | | GGCATTCTAAAGGCCTGTGCTGAGTCATCTTTCACAGGAACCAGCTTCTCAAGTCT |
| | | CTGGGATCCTGTTTTACAGGCTGTTACTAACCTTCCCTTGGCTTCCAGGCCAAGGA |
| | | AGAAGAAAGAATAAATATTAACCAAAGGTACGGCTGTGGCAGGGTGCCCAGGGCCC |
| | | CTCCCTTTCCTTCTCTCCCC |
| 5 | BC3M_125 | CCACCTCTAGACCAAGTGCCTGCCTGGAATGTCCTGTCCAACTTATCCACCAGCTC |
| | | ATCCTTCCGGGCCTAATTAAGGCCCCACTCCATCTCTGAAGCCACCCCATGCTCAT |
| | | GACTCTCCCTGACGCAGGTTCCCGACACACCGGGTGACTCAGCTGCAGTGTTTTTC |
| | | ACAGTCCGTGATGCGTCACAGCTATTTATAGGTGTGCTTAACTCCCTGTGAGGAAG |
| | | CACTTCAACCCCCAAACGCAAGTTCCAGAAATATGCTCATAAAGATAAAGATAGAG |
| | | AAAAGCTCTGGAAAAATACA |
| 6 | BC3M_132 | TCTGGAAGCAAGTTACCCACAGGTTTAGTTTGCCTGGAGAGAAACAGGCCGGAGAG |
| | | AGACTGCGGCCTCCCTAGGGTCTTCTGACGGCAAATTCCTCCAGCTCAGTGGCTGC |
| | | TGGGCAGCAGCACAGCCGGTTTCTCTCAAGGGCACACCCCACACACCGCGTCACTG |
| | | TGCACTAGCCTCAGATGACAGACAAGCCTTTCACAAGACTTTTGTGGCACTGTTCA |
| | | TTTCTGAGACCTTCTCTATGATGAGCTCAAACTGCTTACCTCAGAGAAGAAACTGC |
| | | GTGCACAGAAAGCTGCTGAG |
| 7 | BC3M_137 | TAATTTCTCCGAGGCCAGCCAGAGCAGGTTTGTTGGCAGCAGTACCCCTCCAGCAG |
| | | TCACGCGACCAGCCAATCTCCCGGCGGCGCTCGGGGAGGCGGCGCGCTCGGGAACG |
| | | AGGGGAGGTGGCGGAACCGCGCCGGGGCCACCTTAAGGCCGCGCTCGCCAGCCTCG |
| | | GCGGGGCGGCTCCCGCCGCCGCAACCAATGGATCTCCTCCTCTGTTTAAATAGACT |
| | | CGCCGTGTCAATCATTTTCTTCTTCGTCAGCCTCCCTTCCACCGCCATATTGGGCC |
| | | ACTAAAAAAAGGGGGCTCGT |
| 8 | BC3M_139 | CCTGACTGTGAAAGCCAGGCCCCAGCCCAAGAAGGCTTCACAGACCCCTAGGTGTG |
| | | CCCTCTGTGTGAGCCAAGTGTTGACCCTGGCGATGATGCCAACAGCCCGACTCTGC |
| | | CCAGCTTTCAGCCGCATGAGTGTGAACCAGCTGAGCGGCACCAGCTCAGGGCAAGG |
| | | CAGAAGGCCAGGTGCACTGTCTCTAGGCAGGCAGGATGAACAGCAGCACCTGATGT |
| | | CACAGCGGCCGGGGAACCACCCTGGTTGGGGCATGCTAACCCACCCTGCTAATATG |
| | | CTTTGGGTCCTAATTTCCTT |
| 9 | BC3M_142 | ggctcacaccctccaggggctaccctggtcactcagggtaaaagccacagcccttc |
| | | cagtggccttcaaggccctggtgatctgctcgcccctcccctttccactcacacct |
| | | tgcccccccactcctggcaacccgtctctgctccagccacacacttgcttcattgc |
| | | tgttcctggaaaatactgggcatgttctggcctcggggcctttgcctcttttgtgc |
| | | ctgctgccaggacatctgttcctccggaaagcagcctggatcattcccttctctcc |
| | | ttcagggctttattcaaaaa |
| 10 | BC3M_146 | GGATGAGTCACTGGATCCGTTTTCAGTTCGTTCCACCCACAGATCCGTCCTTTGCA |
| | | GGCGCCCCAGAAAAGATTGCTTCAGAGCTGGCACCAATGGAGAAGGGACAGAGGCC |
| | | CAGCAACAGGGCGGGATTGGCAGGCGGAAGGGAGCGTGTGATGAGCTGAGCTCACA |
| | | AAGGGCCGGGGTGCTGGGCTGCAGCTGGGGAGGGCGGGGTTGGATCAGCGCCTGCT |
| | | CCTCCGCCTTCGTTTTTCCCCTCCCCCTAAGGATTCAGTTCCCCCTTCTGAAATTC |
| | | ACCACCTTGTATGTGACTTA |
| 11 | BC3M_154 | cacctttcccaagatgacgacatacctaattttgcatagcacctgagattgtAACT |
| | | AAGGTGGTGGGAACCCTTGGTGACTTGCTGTGTTGTGTTGGCCAGTGTTAACACTC |
| | | ACTTCCCCTTAACAGCCCTCCAAACCCAAAAGGCTATGTCAAATCCAGTCCCAGTT |
| | | CCCAGTTCCTTGTGACTGAGCCCCTCACCCCGCTGGACATTCCTCTCCAAGCAGGC |
| | | AGTGCTTCCTTATACCCTCCCCACACGGGTAGGTGTTGAGAGGCCAGTACTGAGGT |
| | | AAATTTCTTTCTTATGGGCA |
| 12 | BC3M_168 | GAACTCATGAGTCAGGGTCAGTCAGCCCAGAGCTGCAATGTGTACGTGCTTCCCGG |
| | | CCCTGCTCTTCTGGCCCGCCCCCAAGCCTTCACGCATGCACCCCTGCAGGCACTTA |
| | | CCAGCCTCCTCATCCGTATATCCTGGAAAGGGTGCAAGCATGCCTGGCTTAGTCAT |
| | | CCATCCACAGGAAGTTTGCACAGCCCTACCTGAGTGCTAAGATCAGGCTGTAAACT |
| | | GCCAGAATGAAACAAAAGAGGGAAAATAAATATCAGCACTCTCCCATAAATTTTGC |
| | | AATAGTCAGCTGTAGTCTAG |
| 13 | BC3M_171 | CAGACAGAGGCCGCTGAATTAACCCGTGGAGGCGTCTCTCTGAGCAGAGCCCGCAA |
| | | TGCGCCTGCTTGGGGCTCCCTGCAGCCTCTGGGGGAGGCAGGGCGGCCCAGAGCAG |
| | | GCCTGTGCTGGAAAGGA\CGCGAAGCCCTGTAACCAAGCCTGTACCTCTGCAGTGC |
| | | TAGTCCCAAGGGGCCTCCGAGCTGTTTGTCACCATGTGATTGGCTCAGGAGAGGGG |
| | | TGGAGAAATGAAAACACTCTGCCCAGGATATATTTAGTTGAAGTGCAGCTGGGGAA |
| | | GTGCTTAAACAAGGGAGCTT |
| 14 | BC3M_172 | TCTTGGATTTCTGAATATGCAGTTCTGTTCCTAACCAGTGTGTCCCAACCAGAAAG |
| | | TCACTGTAATTTTTGGTTTTGTTCCCAATCTCCCTCCAAATGTCATTAGTCATATC |
| | | CTCCTTCCCATttctgccttgaataggcagtcattatgatgaagccaggcttgttt |
| | | cagaattccatgagaaccacagTGTCAGGCTGTGACaactctggggctggaaatgg |
| | | aaaaggctgtgatctggggttggctggcaccgtccccgtgagtcattatggaaaca |
| | | ctgtccccggattctgctga |
| 15 | BC3M_173 | aTTGGTGAGGCGCCGCGCCTCGGTGTCGCAGCGAATCCGCAGATCCTCAAGCCAGG |
| | | TGGGGGCGCCCACTGCGCGTGTGCAGCGCCTGATAGCCAGGCTAGCTGAGGGCGGG |
| | | GAGCAGCTGCGGCACCTGGGACACAGCGATTGGCTGGGACCAGGAGAGGGCGGGAA |
| | | GAAGAACTTGGCGGAGCGCGCTCATATCTCTGATTGGCTGCCAAGGGTAGCCCTTG |
| | | ACAGCTGCCGGGTGGGACCCGTAGACCGCGAGCGCACTGGCCCGTGATTGGTTGGG |
| | | GTGCGGCGGCGAGCATCTGC |
| 16 | BC3M_178 | agcacttcccgggcgccccgcctcagtttccccatctataaagtggagatgataat |
| | | aGCATTCAGAGTCACTGATCTAAGGGCTCAGGGACACCATTCAGTGTAAGCCCCAT |
| | | ACACTCCCTGCAAGAGGAAGCTGGTTCTGACTCAGCCTTGAGGCTGGCGTCTGAGG |
| | | CAACCACAAGCCCAACGTGCATGGTGGAAAGATGACTGTAAGTGGGGGCAACCTCA |
| | | GCTGGCCTTGGGTTTGACCATGGAATGCGAGGCACAAAGGGGCCCATTTTGCATAC |
| | | TTTCTCAGAGGCTGTAGGGC |
| 17 | BC3M_179 | CCCCCGACACCACCACCTCCTTCTTCGCCTTGCATCGGTACGATAAGGCACTTGCT |
| | | TGACGGGAAAGAGAAACTCAGCTGCCAGCTGGGGTTCATTTGCACTTTCCCCCGCC |
| | | TGGTCTGCGGTCTGGCTGTGCAGCTAGCCGCTCTGACGGGGAGGAGGGGCCCAAAG |
| | | CCACTGCCTGCCGCCTGGGCAGGGGAGAGGGGCACGTGAGGCTCATGGCAGAGGCA |
| | | CAGCCAGCTTCTTGCATGTGCCCTCCCCGGGGAATGTCTGCAGAGCCCAAGACTGC |
| | | CACGCCGTGGGCACAGCCCT |
| 18 | BC3M_182 | GGGAAACCTTGCAGACTGTGGGGTCCTGCACACCTAGACTTGCTCCTTTTAGAAGC |
| | | CATGGAGGAGGTTGATAATGGGAATaacatttattgtagcttatctctatgccttg |
| | | agcaatgtgctcacactggctggttccctcctcacatcagcctgatgagtcagatc |
| | | ctgttattacttctcactttacagatgaggaaGTAGCAGTAAATCCATTACCCTTT |
| | | TCAAGCGGAGGTTGCAAGAGGTTGCAAGCGGAGGCAGAATAAACACTTGAAACAGt |
| | | gagtcagatcctgttatcac |
| 19 | BC3M_199 | GGAACCCTAGGATCTGATTTAGGACATTTGGAATCTTTAAGGCACATTCGATCTAG |
| | | AAAGTGGAACTGAATTGCTTTGGGAAGGCAAGAGGATGATTTTACAGTATAGGGTT |
| | | TGTGTGGAAATCCCCTTCAGCAGTAATCAACCCAGGTGTCCAACCTGTTTGTTAAC |
| | | CATTTCCAAATGACTCAGAGGACCTAGAGGGAGGGCTTGAACACACTCCAGCACTG |
| | | TTTCTACAATTTAGCCTTTATTTGCATTGGAAACCACATTCCTGAATTCTTGAGGG |
| | | GGCAGGCTCTGGCTTATTCT |
| 20 | BC3M_20 | tcgTGTGGGCCTGGGCCGCTTGCTATTACTAATAAAACAGCAGCAACCACAGGACA |
| | | GCTTCACTTCCGGAAACTCCCTCTGTCACGTGCTTTGCATGAATCCTCACACCGTC |
| | | TCACTAGGGGCGCTCTCCCCGTTTCACCAGTGACTTGGTGACAACCAGCCTTGCTC |
| | | ACGAAGCGTCAGCCGTATCCTTTCTGTGTGCAGTGGGGTGTGGGTTGTGTGGAGCC |
| | | GCGGTGTCTGTGGAATTCACAGGCTGGGGCCGGAATCCATGGCCCCCGTCGCCGCT |
| | | GCCACCCCCCAGGTGCTGGG |
| 21 | BC3M_203 | AGGTGGTGCGCCGGCGGTTCGCAGCTGCTGTGCCCGCTGGCCTGGGCGCAGCCGGG |
| | | GACAGCGACGCGTTTCCTGCCCGGGAAGGGCCCGAGCGCAGGGCCGGCTATAGCGG |
| | | TCCCGCAGCTGCCTGCTTCGATTTTAGCACTGCTGCTCCCTAGAGGGAGCAACGCG |
| | | GCCCTCTGTCCCTCGTAGGGCTTGAAATGTAAATTATTCATATCAGGGGAATGTGT |
| | | GCTTCAAAAAGCAAGCTGGACAAGAACCGACGGGTAATCCTCGCCAAATTCTTCTA |
| | | TTTAACCCTCACCATTAAAA |
| 22 | BC3M_206 | TGAATGTCATGAGTCAGGAAAAAAGAATTTGAGCGCAGTCTGGAAATGAAATTTCC |
| | | TGCCTGTGGTTTGACTCACGTCTGTCTGTCTCGAAATCTACCCCAAGGACATTTAT |
| | | TCCACTGTGACAGGGCTCATCTCTGAGGAGCACCAGACTCCTGCGGTGGGGAGGGA |
| | | AGATTATCCGCGCTGCAGAGACTAGCTGGCCTCCGGAAGCCGCCTCCTGACCCCGC |
| | | GTCAAGCACCGCGGTGGATGGCGCAACCCAGCTTTGGGAATTAATTACCCAAGGCG |
| | | CGTTTCCGTGCAGTCTGGCC |
| 23 | BC3M_212 | AATGCCCTGCCCGATCCAGTTCCGGCCTCCCATCTCCCCTTCCCGCGTCTCCACGC |
| | | TCTTTCCTTCCCCGGTTCTGCCGTGAATGCTCCCAAGTCCTAGAGCACCGGAACTC |
| | | CCCGCGCGCCTTGGCTCCTGGGCCCCAGCTCCGTGCAGTCCTGGACTGGGGCTCCA |
| | | GGTCCACCAGGGGGCGCCCGCTGCCCAAGCTGGGTATCGCTGCGGAGAAAAGGGGC |
| | | CCAGAGTGATTGTTCCTCAGGGGAGGGAGGGGGAGGTCCCCAGAGGGAAGGGCCTG |
| | | AGTTTCCTCTTGGGGGATGG |
| 24 | BC3M_22 | aaactaacagggaatggtgttgccacctgtagccccagctacttgagagactgaag |
| | | caggaaaatcccttgaagccggcaggcaaagattgcTCACTACAGTCTAGTCTAAA |
| | | ACCCCACTTCCAAAAAAYTAAAAAACGCACACTCACACCATTACAACAGCCCAAAA |
| | | TAAATGTTCAAACAAAATGTTGTCTCACACCTCGCAACAAACACACAACTTTCTAT |
| | | CTGATTTTTAAACACCGTTGATGaaccccaccaacatagggcttcaaaaaatttgc |
| | | ttgaaactcaaaacggtttc |
| 25 | BC3M_221 | cccaaagtactgtgatgagctactacgcctggtcATTGTCCCTCTTTCTCATGACT |
| | | CTCTGGACATCCCTGGGGTGGAGGGTGGGGCAGGCACACACATCCCTCAACTTCCC |
| | | AGTGGTTCCACGATGACTAAGCCAGCCCTGTCCCTGAGGCTGGGAGTCTGGAGCTA |
| | | GGATCCACCCCCATGGCCTCATATCCCAACCTTGAGCCTGGGTTTCTGGTCAGACT |
| | | GGACGGGCTAGCTCGGTCTCCTTAACTCTCAGAGTTGCCTTGTCCAGGCCCAGCGG |
| | | GTCCCACACAGCCAGGCACA |
| 26 | BC3M_224 | ttacccaagatcaTTCGGTGCGGCCTCAGCGCTGGCGCTGAGTCCTCTTCTGCCCC |
| | | ACCCCTCAGGCTCCCAGTCCTGGTCTAGATCCCTAGCCACGTAGCGTAGAAGGGGG |
| | | CGTCGACGGGGGTTGGGCTAGAGTTGGAGCGGGGAGGAGATGAGCTAAAGCGGGGC |
| | | TGGCTGTGCGAGAGGCAGTAGCAGCGGCGTGTGTCCTGGGGCGCCCCCCGGTGGCC |
| | | TGTGCTGGGGTCGTCGGCCGGGATCCCCTGTTCGACGTACTCCGGGGCTGAATGGG |
| | | AAACAGACAGTCCCAGACCC |
| 27 | BC3M_226 | aaaaaagaCTAAGTGGAGATGAGGGTTCAGTGCACCCCCATCTCCTGGCCCTGCTG |
| | | CCCATGAGCCAGACCCTGAGCTGACAGATTGGTGCCCATTTCCTCTTATGGATTGA |
| | | TACGGGGCTCTTACCTCTGGGTTTGCTCAGCCCAGCAGCAGGCAGTCAGAGCCAGA |
| | | AGTTGTTTGCAAACCGAAACCGGTCTGCGGCTTGGGCCACCTACTTGTGAAACCAG |
| | | CTGTCGCTGTTTTTCCTCCCTGTGAGAAAGTCCCCCAGTAAAGCTGCGCGGGGGAG |
| | | GAGAAGGAGGGTGGAGGAGG |
| 28 | BC3M_230 | AGGGGCAGGGCCAGGGCGGTTGGTGGACTGGGCCTGGCTGTACGTAGGTGCTCTGA |
| | | GAAGCCCCCGGCGAGAGGGGCGGGGCCAGAGCAACAGTGGGCGGGGACAGGCTGTG |
| | | CGTCGGAGCTCCGCGGGGCCTGCGGCGGGGTGGGTGGGGCCAGGGCGGCGGTGGGC |
| | | GGGCCGTGCTGTGCGTAGGGGCGCTGAGAGGCCCGCAATGTGAGAGGGGCGGGGCC |
| | | GGAACAGCGGTGGACGGGGTCTGTAGTTCAACTGTGCCGTGGCGTCTTCTTCGCGG |
| | | CGAGATCTGAGTGCCTCGCA |
| 29 | BC3M_231 | GGAGCGGTGCAAAGGTTCTTATCCTATTTATCGGAGCCAGTGTCCAGAAAAGGAAG |
| | | CTTGTGGTTTGAGACATTCTGTAAATCCGGTTCCAAGAGCACGAGGTAGGACTCTG |
| | | AATCCGATGTGGTTTCTGTTCTCGGTGATGGTGCAGAGCTGTGAGCCAGTGGTAGG |
| | | GTGTCCTTTAAATTCCAGCTCAGTACACTAGTTAATGAACTTGGCTGACTGATAAA |
| | | AATGTTTTCAGGTTTAGCTCATGAACATATCAACATAGACCTAAATATAATTCCAG |
| | | TTTGTCATGAATGTTGATTT |
| 30 | BC3M_232 | GGCCTCTTGGGGGCGCGGTGAGTAGGTGGCCTCTCCAAGCACCACTCCCGATGTGC |
| | | GCATGAGCGCAGCCGCCCCTACGCAGCGCGTGCGCACGTGCACTCACCACGTCCAT |
| | | CCCAGACGTGCGGACCCGGGTGTCTGCAAGGTTCAGTCTCCACACCCCAGCGCCCG |
| | | ACCCTGCGCGGGGACATGCGCACAAGCGCGCGTCCTGACCACCCGGACGTGCTGGC |
| | | CCACACGCACACGCGTGCGCATTACCCCCGCCCCATCCGCGCCTGCGCTCAACCCC |
| | | GCCTACACCTGCTCCGTGGC |
| 31 | BC3M_235 | GGTCCTGGACCGGGACTTAGGTCCACACCCACGTGCTGACGTCGGGCAGGCTCAGC |
| | | GGCCTCCCGCGCCTGCGCAGCACCGCCCTTTTCGGGCGCGGCGCCCAGTCCCTACA |
| | | CCCCACAATCCCCCGCGCCGTTCCGGAGGCGCGCTAGGAGTGGGTGTGGCCTCTGC |
| | | CTCCACATTGGAACAAGGTGAGGCAGAGGGTGTCGCGTGGTCTTCTGGGAAATGTA |
| | | GTTCGTCTGCCAGGCCGGAACCACCGCTCAACCGGCTCGCGAGACTATGCACCCCA |
| | | CAATGCGCCGCGCGCGCAGC |
| 32 | BC3M_239 | TCTAAGTCTGTGCATGCATTTGTGGTCAGAGTCTGGGGAGCTGGGGGCGTGAATGG |
| | | GCTGCTTCAGACACTGCTTTGAGGGTGTGACCAGGACCTGAGGGTGTGGTTAAGGT |
| | | GTAGGGGTGGGGCTAGGCCCTTGGGGGTGGGACCACAGTCCCAGAGGCGTGGCCAG |
| | | GGCCTCGAAGGTATGGCCATAGTTTGAGGCGTGGCCGAGAAACTCCGTTCCCAAGG |
| | | GAGGTGGTAACTCTGTGCTCAGAGCGCCCTCTTGTGGCTATCCTCAGGTCTCCACT |
| | | TTTTATTCAATAGCTTTATT |
| 33 | BC3M_241 | GTGGCCCGCTGTAGCCCCGCCCCGTGGCCCGCCCGCAGTAGGCCCGATTCAAATCT |
| | | GGCCAATGATAGTGTGTAAACAAACCCAGGCCCCGCCTCCCGACGAATAATCCCCC |
| | | GACCGGCGAGAGGCCCATTTAACCCGATGGGGTTTGGGGTTGGGACGGTGATGGAG |
| | | TCGTGGCTCCGCCCCCAGACCTGGGCCAATAGGCGGCTGGGCTCCGCCCCCGGCAC |
| | | TTGCCGCGCTGAGGACCCGAGGCAGGGCTGGGCGCGCAGTTGCCTGATTTCGTGGC |
| | | GGCTCGCAGTCTGGGCGCTC |
| 34 | BC3M_245 | cctccaaaagtgctgggattactggcgtgagccaccgcgcccggccTCAGGGCGCG |
| | | CTTTTAAGGAGAGTTCCTGACATGACGGTGGGCTTTTCCTGCAGATGCACCTCTGG |
| | | GTAGCGCCCTCTTTACAGCCTTGAAACCTGGTCAACTACATTACTCAGAAAGCTCT |
| | | GCGTTGAATGAATGCCGTCAGAGCCAATGAGGGCTCGGAAAGAAGCATTTCCGTGT |
| | | GTGCGCCTAATGTAGGGCCGAGACTTCCGGGGTCCTCTTGTAGCGGCCACGTTGAT |
| | | CTGCGATACGCGTGTTTGCC |
| 35 | BC3M_247 | ACAGCCTTTTGGAAGTCGCGCTAACCTTGGCCTGAGACCTGCAAACTTGCCCAGGC |
| | | TGGGGCGTGTGAACCGGCGAGCGCGCAGCGGAAACGGGGCGGGGCACCTGAGGCTG |
| | | GGAATGCAGAGGAGCCTTCCGGGGGGCGGGGCGGGGCCTCCCGTGCAGACCAATGG |
| | | TGGAGTAGATGCAGATGTCAAAACGCGCGCTCAAgtggcttccgccaggaatcccg |
| | | acgcttagggaggcggagggaggatcgcttgagaccagcctgggcaaacaagcgag |
| | | accctcgtctgtttacttaa |
| 36 | BC3M_250 | AGGCTCCAAGGAGTTCAGCATAGCACGAGCTTTTAATTTGCGTGCAGACAAGCACA |
| | | AAAGGCACAACCGGATATACCTGTTATTTCCCAATGACCTGAGAGCCCGAAGTTTA |
| | | TGTTAAGCCTTGGGTTATGGCACAGCTTGCACGCAAGGCCCTGCAGCTCCTGCAGG |
| | | CAATTGAGAGGTGGTGGTGTACAGGACAGAGGAACAACTCTGAAGTGACAGCACAT |
| | | AATTTAATTCCCCCTAAGCTTTCCAAGCATGCAGACTGTTCCTTTTTTGTCAGCGT |
| | | ATAACCTAAGTGATTTGTTC |
| 37 | BC3M_252 | aacacacacaacacacacacacacacTCTTTCAAGGTCTAGCAAAACCCATCAGGA |
| | | GAGGTTGGGCCCTGGAGGTGCTGTGGCTTCCTGCTGCCCCGCTCCCTCCCGCCTCC |
| | | TCCCTGCAGGGCTCCTCCTGGGGAGGCCTGTCCAGCTGCCAGGCCCCGCCCCGCCA |
| | | CAGCCCCCGCTGTCCTCCTCCCTCCCTCAGCCGTGCCAGCAGCGGCACAGAACTGG |
| | | AATTGCCCTGGACGGCCACAGCTCTGCATATCCCCCAGGAGTGTGGACAAGAAAAA |
| | | ATAAACACAATTAGAGTTCA |
| 38 | BC3M_253 | AAGAGAAGCCTGTCAGTCCAGCTCGGGCTACACACTGGGTGAGCCATGCACCACCC |
| | | AGGAATTTCCAGGGCACGTGCCACGTAAGGGGCACACCCGACAGAGTCCAATGGGG |
| | | TTCCCCACTGGGCCTCCCACTGAGTTGCTCAGCCTGGGCCGGAAAAGGGTGAGTCA |
| | | CCCTGGGGGTGGGGCTCTCCAGGGTAGAGGCCAAAGGAGTGACTACCATGACAATT |
| | | CTCCGGAGGGCCTGAGGCGGCGGTGGACAGCCCCGGCAACAGTGGGCCCTCCCCGC |
| | | AGAACTGTGGTTCCAATCCC |
| 39 | BC3M_255 | gcgtgcttgtgtgtgggtgtgtggtggggtatgtgtgtgtCCGGGGCTGCCGATTC |
| | | AACTGAAAAACAAAAGCGGCTCTGAGTCTGAAGCTAAGGTTTAACAAGTGACCAAG |
| | | ATGACTCATGCTGCTTGGCTGCAAAGGCCACAGGGCTGCCACCCCCAGCGGGGCGG |
| | | GGCCTGGGTGGGAAGAGTCACAGGTACAGAGGCTCCTGTGACATTCACACTCTGCC |
| | | CCTGCATCGGCTGCCTTTGGGGCCAAATACTTTTGTGAAAATTAAGACAGAAggcc |
| | | gggtgcggtggttcacgcgt |
| 40 | BC3M_257 | AAACCTGCGGGCCCCGGTCCAGGCGTGGTCCCGCTCGCACGAGGGAGCGGTCGCCC |
| | | AGGGTGCCGGGAAGTCGGGGACCGGCCAGCCGCCGACCGGCCGCACCCCTCCCCGC |
| | | CGAGCTCGCGCGCCCGCCTCGTCAGCACCTTTCCCGCAGCGCAGCCCCACAGTGGT |
| | | CACGAGGCGGGCGCGGCCCGGTCAGCCCTGGCTAGACTAGGCATCGGCACCACCCA |
| | | CCTCGCCCCTCCCCGTCCCGCTGGTTTcccctccccctccttcccctccccctctc |
| | | tgttctccttcccctcccGATCCCCGGGCGGGCCGCAGCGCGCCACGTACCTGGCC |
| | | CCGCCCCTGCGAGCCACGCAGGGAACCCCGGTGACGTCACCACCCTCCGGCGCTCT |
| | | CATTCCCG |
| 41 | BC3M_260 | cctgattagccagaactataggtgcacaccaccacgcctggctaatttttgtattt |
| | | ttttgtagagacagggtttcaacatactgcccaagctggtcttgaactcctgggct |
| | | caaatgatccgctctccttggcctgccaaagtgcagggattagaggcgtgagtcac |
| | | cacgcccagcccattttccttttcctgtccataaattcctctctgaccacatggca |
| | | gcatcagagtccctctggttcagggagttaccggattcatgaatcattctttgctc |
| | | aattaaactctgttaacttt |
| 42 | BC3M_265 | TGTTTCTAGCTAGTTATAATTGGCAGGCAACCAGAAGCCTCATCTGCCAAGGGCGG |
| | | AAGTCATGTCTGGAACAGGTTTCCCTCTTAAGACTGTGGGCTAACCCAGCATCTTG |
| | | CCACTTTGTGTGGGACTTCCTCATTCTTAGTACATAACTGTGTTTGACCCTCAGGG |
| | | ATGACTAGTGTTTCCTGGCCTCGGTACAGTTGACTTCTCCAGAAACTATCTGGCTC |
| | | ACTCTCAATTTCCTGGAGCCGTATATCCTAATTACAAAAATGGGAAAATCATACCT |
| | | AGAGTCCCATAGAAAGAGAA |
| 43 | BC3M_266 | AAGGAGAGATGATGGAGGCAACACTTACAGGTCCTGAAAACTGCTCAAATAGGCAC |
| | | AAAGGAAACGAAGGATGCCTGAAAATAATGATGATGCAAAAACTAAGCTAGGTAGG |
| | | GCAGCAGGAAGAACCGGTTTGGTGGGAAGATGATGAATTTGGCTTGAGGTGCTTGG |
| | | CAAGACATGCAAGTCTGCTGCACAGGCAATGCAGGTCAGCAATTTGAGAGAAAGGT |
| | | AAACTTTCACAATCCTAATTTGAGAAGCAACAGCACGGAGATGATTATGGAGCCAT |
| | | GAGGGCTGAGACACTCAGCG |
| 44 | BC3M_267 | CCCATGCAACTGTGTGATGAAACAGCCCCACACATCCGGGAGCACAGCCAAGGCGT |
| | | CCTGTGCCACCTCCCTGGTAGAATCTGGCTTTTCAACTTGCTCACCCATGAGAGGA |
| | | AAGCGGTTTTAGACATCAGGCTTACCCCTCTCCTAAGCCACACCCTTTTCTCATTC |
| | | CCAGCTGAGGAACTGAGCCTGAGACACTGAGGTTCCCAGCTGCCTCCATGATTCGC |
| | | CAGCACCCAGCTTCAGTTTCACATCCTCCCAATCGTCATAGCCAGGACAGCATGCC |
| | | TCACTGACCACGAGGGAATG |
| 45 | BC3M_268 | AGGCGCCGCCGCTGAGGGCAGGCAGCCCGGCAGCCACTACACACGGACCCGTGACG |
| | | rCGGGCGTAGCGCGGCGCACGTCACGGCCGCTCGCTCGTGCGCGCGCACCCCTCCG |
| | | CCCGGCGGTAGCGGAAcccgccgcgggcgcgcgcccggcccAGGGGAGTGGGTCGG |
| | | CGCCTGCGCAGAGGCCCGCCACGCCCACACACAGGCCACCGCCCCCACCGGCCGGA |
| | | CGGCGCGGGGATTCCCAGTCCTGGCTCCGccccggcctcggccccgcccccgcccc |
| | | tgccccGGGGCAGCCTGTGCTGTTCCGTGTGCGCGGCGCATACGCACCTGGGTTGT |
| | | CTCGAGCCTGCGGTAGTGGCCAGATCCCAGACATCCGAGTAGATCCCGTGAAAAGG |
| | | TCTCCCAC |
| 46 | BC3M_269 | ccagccactgtgagtactggctgctcctgactcacagctgcaccctttgagggagt |
| | | gaggggcgttacccttggctgacaggatatgattagaaagcctggaaggcggctgg |
| | | tggtggcccatggccaatgagtcactgtgcgagtgtatactagcccagccctcttg |
| | | cctccaggcaggaaaacctctgtgtgaagtgctctacttgctccatgctctggcgc |
| | | tctctgtacctacgcaggctgaagctgagcctagacatctcctgaaaccacacctt |
| | | tgactcgcttcttccccttc |
| 47 | BC3M_27 | AACTTGAAACAAATAAAGCAGGTTGAAGATCACAGTGTGTGCTGCTGGGCCTGTGG |
| | | GGGCGCTGGGCAGCAGAAAGGCACACTCTGCCTGCAGCCTCGGGATCTGGTCGCCT |
| | | GTGTGGGAGTAGGGAGGAGTCCTGACGTACCCTCTCTAAGACTGGCTGCTCTGCAC |
| | | CTCCCTCCAAGCCAGGCTGGCCAGTAAAGAAATCTAGCTGTGGACAGGAAACGAGT |
| | | GGTTTTTGTGATCTGAGCAGAAAGGGCGTTTTAGGCCTGGAGCAGAGTGGAGGCCC |
| | | TGAGCCACGGCCCAGGAAGT |
| 48 | BC3M_275 | tttgtgtgcatgtgcgtgtgtgtCTGGGGGAAGGAGGTAGAGGAAGTGAGATGATG |
| | | GTGACAGTGACAGCAGCTTGGAGAAGACAGGGGGGTGGGTCTACTTCTGAGGAAGT |
| | | CCTTGGCTGAGGTAGGGCCGCAGAGAGGCAGGGTGAGGGTGGAGCCTGTGGTTTCA |
| | | GAGAGGAGTTTTAATGGCTGCCAAGAATGTGCACATGAAGCCGAAAGGGAGTGCGG |
| | | CCTGGAGCTGCAGTCAGCCCAGAGGGCGGGTGGAGCCTGTCCCAGGGCACTAGGAT |
| | | CGCAGAGAACGACAGGAGGG |
| 49 | BC3M_277 | AAGGTATTCGAATCGAATGAAATGGAATCGAATTGAAGGGGTATgaatggaatgga |
| | | atggaatggaatcgaatcgaatttaatggaattgaataggaaagaatcaaatggaa |
| | | tggaatcaacccgagtggaatggaatggaatggaaaggaatggaatggaatggaat |
| | | ggaatggaatggaatggaatggaatggactccagtggaaaagactggaatggaacg |
| | | gtttcgaatgaaattgaatcgaatgaaatggaatggaatgcaatggaatcaaatgg |
| | | aatggacttgaatggaatgg |
| 50 | BC3M_283 | CCCATCGTGTTGCGAAAGCATTCAGGTTGAACAGTGTTCAGGAAGAATACTCAAGC |
| | | AAAAACTGGTTTGCAGCCAAATAcagagactgcaaaccccagtggcttcaggggcc |
| | | aggcagggaaagtaaacatgtgaaacaatagggagtagtcctgcctgtggggaaca |
| | | ggggagttctcatgccccagcctaaTAAATGAAAAAATTATTTATACACCACAGTG |
| | | GAACCGGAGATGCACCTAAAGCCATTGGGATGTGGTTtctctttttcatctcactg |
| | | ctctgtctctgatgtggctt |
| 51 | BC3M_284 | AGCTTGGATGCTGCACCCAGGACTGAAAGGGGGACCTGTGGGCGGCCTCTGCCTCT |
| | | CCCCGCGCAGCGTCAGGACACAGGCCCACATTCCCTCCTGGCTTCTCCCTGAAGGG |
| | | AGAGAGAATAATAGTTGGTTCAAATGTCAGGCCTGCTCCGTGCTGGTGGGGAGACT |
| | | GGTTGAGCAGGTCCGCAGGAGGGACGGAGGGAGGAAATTATTAATAATTGCAAAGC |
| | | AACCAGCCACACTACAGGCCTTGAGTTGTGTCTGCGTTTGTCTTTGGAGGTGTGGA |
| | | GTTGGGGGTGCTGATCCTGG |
| 52 | BC3M_290 | CTGGGGGACTGTTGGGTCAGAAAGTGTTCAGGGAGCAGCTGTTgcgccctccctcg |
| | | gccccgccgctcggagacgccccgccccctgccttcaccggccgccccgccccctg |
| | | ccttcaccggccgcccggccacgccccacaccgccccggccccgccccagcgccca |
| | | cgtgactagcataggcgcgcccctgctccgccccccgccgccgactccgcctccgG |
| | | GACGGGAGCGAGCGGCGAGCGCGCGCACTCCCAGTTCTCGCTCGGCGACTCCCGCG |
| | | CACGCGCGCGCCGTGCCACC |
| 53 | BC3M_291 | CTTTTTCCTTTAAAGAATACACTTCTTATGTAATTTGTTTTGCATTTCTGGAATGA |
| | | GGAACTTTTCTGCTCATATTGTTGTTAAAATCTAGACAACACGCCCGTGTGATAGA |
| | | TCACCCTGAGCCTTGGAAGGAAATGATTCACCACAATACTGTAACTGAAAGTCGTC |
| | | TAACACCAGGGCTGGAAGGCAGGCTATGAACCGCTGCATTACCTGCGTGCAGCAGC |
| | | AATGGGAGGCAGCCAGAGGTTCCCTCGGCCTGCCTAGCTCACTTCAGCTTTGTTCC |
| | | TGTTCTGTTTCCTCCGTCCG |
| 54 | BC3M_292 | CATCAAGGGACCCAGAGATCACAGAATAGCCAGCCCTTCATTTTCAGGTGAGGGCC |
| | | TCTGTGGGAAGGTGCGTTCCAAGCCACACAGTTGGAAGTTGAGCGAACTGAACCAA |
| | | GGCTGGGCTTTTGTGTTTGCTGTTTAAACAGTGTGTGGTTTTACTCACCTACCATA |
| | | GTGCTCCTCCTACTGGTGGGCACCTTAGAGTAGGCTGAAAACAACGTGTCTCACTG |
| | | TCCTTTTTTGTTTGTCTCTGAGTATTTTTCCTTATGATCTTGAAGTAACATTTACT |
| | | TAATTTGCAATGAATGAAAA |
| 55 | BC3M_295 | TAGCAACATGAGGCAACCTTGTCTGCGAAAGAGGAGGTGACCGCAGCTCCTGGGGA |
| | | TGTGCCAACTCTGGGATGTGACGGGAAGACAAAGGGCTTCTGTCCCCTTCTGCCTG |
| | | GCGGTAAGAGAGCCGGCCGCCCGGCAGGCATGCCCCAGCCTGTGGTTCTGGAATGC |
| | | GGGCAAGCCACCGTCCCCAGAGACCTGTGTTGGTGGCCAGGCCAGCCCACACACCC |
| | | GATTGGCACATACTCTTGTGCTTGCCCAGGAGCGGAGTCAGACCATTCACGCTGCC |
| | | TTCATGGGAGTTGAACAGTT |
| 56 | BC3M_307 | TGCCCCCACATCGCCATCCTGCCTGTCCTTCTGGGCCTGCACGTTTGTTGTGTTTG |
| | | GAAGGAGCCACCAAGGAGGAGGATGTCAATGTGCAAGTTCTCAGGGAAGCAGGCCC |
| | | CGCAGCCTCCGTCAGTGTCTTCCGTCCGCAGGAAGAACCCAGGCCTGGGTGATTCA |
| | | TCGGGGCCTCAGGGCCGGGAGGCACTAAATCTTCTGCAGATGTGGTAAGATCCTAT |
| | | CACAGCAGAAAGGGAAGGGCTAGAGTCTCAGGGAAGGTTTTGCTAGGGAGACGGGC |
| | | TTGGAGGGGGCTGAGGCTCA |
| 57 | BC3M_321 | caaaaaatactgagcacaaataaatattcaCTGTAAGGCAGGAGGCagccgggacc |
| | | agactccagatcagatcgaagactggcggaaactgaggagaggcgcttaaagcccc |
| | | tctccataagacacgcccaccacctccatgacagtttaccattgccgtggcaacac |
| | | ccggaagttactgccccttgccgcggcaacaccggaagttcccgcccactttctag |
| | | ctaattctgaatgacccgcctcttaattagcatgtcttttaaagtggacctaaata |
| | | cgcctacgaaactgccccta |
| 58 | BC3M_323 | tggtctctatctcctgatcttgtgatacgccggccgcggcctcccaaagtgccggg |
| | | attacaggcatgagccaccaggcacggcTGAACAGGGTTTTTTTAAAGTTCCTGAA |
| | | CTGGGTGGCTGCCCACAAGAGGGCACTCATGCCTCTGCGTGTGAGTGTGGAACCTG |
| | | GTCGACTGCTGTGACACTCTTTGGGAAGACAGTCGGCATTTTCCACTTCCAGCAGC |
| | | AGGTGGCAGTATGGGCAAGAGTATCATCACCCATCTTTCATCTACCACCCATGTGC |
| | | TTACATCTGGGCTGCTGAGA |
| 59 | BC3M_326 | CTCACCCGTAACACACACACACACATGCGCGCCCTCTCCTCTTGCATGACTCCTCT |
| | | CTCAGGGCTGAGCTGTTTTTCTGAGGGTGCCACAATGAATCAGCTGCTTAGTCATC |
| | | TCTGGAGTGCGGGAGCTAGCAGAACAGCAAAGAGGCATTACAAACCCAATAGCGGG |
| | | TTTCACTTCCTTGAGCAGTATTTATTCTGCTCTCTACCTCATGCTGCCCAAACTGT |
| | | TGGAGAGGCCCTATCCACTCTCCCTGCCTTTTCAGCCCTTATTCTCCCAAATGCAG |
| | | CCACAGAGGAGGTAAGAGAG |
| 60 | BC3M_334 | TTTTTGTGGTTGAGTTCTGAATTAAAAAGTGTCGTACTATATATTTGTTTGGTCAT |
| | | TTCTATGACTTCAGCACTCTCAAAGACTTGGACAGAAGCATAAATAAGAGGCAGTG |
| | | TGAGCATTCTCCAAGTAATCATTCCAAGTTGGTGAGTTCATACTCCACCTAGACCT |
| | | CATGGCCTCGCCACTCTCAGTCAAACTGGTTTTTGTGGTTGTCAAAGTCCAACATG |
| | | GCAAATTTCCCACTGATACTAAGTGAGTTGAAAACTCAAGTTACAGTTGATTTTGC |
| | | CCTAGGGAATTTTACCAAGA |
| 61 | BC3M_353 | AGGGCATTTGCTGAGTTTTGCTTTAtgtgactggatgggactggccttggagacac |
| | | taataagcacgtgagggtttttggacaatgcgaagagttggtgccaagccacaagt |
| | | gggagatgttgaacttcctgcgaatctggtgtgttgtagcctgagtcggtttcaat |
| | | atgaaaaataagagtgacagtgccttccttgtatgctaatctggcgaagtggctca |
| | | tgctggCCATGTAACAACCTGGCAGCCTCCTACAGAAGCAAGTGGGGTGTGGCATT |
| | | CCTGCTGTCTGCATCTTCTG |
| 62 | BC3M_360 | TGAGTGAGCTGGCAAGGGAAGGAAGGTTGGTGAGAGTAAGTCGTAAGTATCTTTTT |
| | | AGAAAAAGAAAAAAAAAAAAAtagcagaggatggtttcgatccatcgacctctggg |
| | | ttatgggcccagcacgcttccgctgcgccactctgctCTATACGGTAGTGATATTT |
| | | GCAGTGAATTCTTTATGATGTTTTCCTCAAAACTTGGTGGGGATTCTGGTTTTTTG |
| | | GTATGGTTAAACAAATCTGATTTCCACACCCCACCAAGGGCCACTAGTTCTATTTA |
| | | TGCTGCAAACATGAGGATGA |
| 63 | BC3M_362 | agacactcgtgccctcaagaacttacaatttagGTTTGTTTGAAAGTTAACTGAGA |
| | | ATTCCAAGTCTAAGGGTGCTGGTGAGAGTGGCCTGGCAAAGCCAGCCCAGGAAGAG |
| | | CTGCTGAGCAGGTTGTA+GGAACGAGGATGCCCCACCCCCCCTCCTTGGCAAAGCA |
| | | GAGGATGGTATTCCAGACAGGTCACAAACAGCTCAAGCAAAGACGTGGTGACAGGG |
| | | ATGAGGAAGGCACGCTTGCGGATCGCTAGAATGGAGGTTGCCTGGGCACAGACACC |
| | | TTGGAGGATCCGATTAGCAA |
| 64 | BC3M_367 | GCAGCACCCAGTTCAGAACTTTGCAGATTGCTGGAATTGCTGGGGAGCTGCCAGAG |
| | | GGCTTTCAGAACTCAGCATGAGTGCAGTGAGTGCGGCAGCCAGCTCCCAAAGGGGA |
| | | TGGCCTCAGCATAGTTTCCAGCTCTCGGCTCTCTTAACAGGAaggcgttgcggtgt |
| | | cgcagacacaatctgaagtgggggttcaaacagacacaacttcacatactggtttt |
| | | gcaacttgctggcaaatgagtgaattttactcaatcccaatttttctcatctgtaa |
| | | aacagccataaaatcgaccc |
| 65 | BC3M_37 | CCTTCCCTCGACCTCCCTTCTACCCCTTCGCCTTAGATGGAGATTTTCTCTTTCTG |
| | | AACCCGGAACCGCTCCCTCCTCCCCGCCCGGCTATAGCTGGCAGGACAGGGATTGG |
| | | ATGCCACGGCCGGTGCGAGCCTTCGCTCTCCGCCGAGGGTAGTGACACAGGCGAGG |
| | | ACGGGCCCCGCAGGTCACATGAGGGCGGGGCCTGGCGGGCTCGTGACCTTCCCGTA |
| | | GGCGGGGTCCCTCCCCTCCCAGCTCGGGCCGACAGCGTCGTCACCAGCTTTTATGG |
| | | GGCACGTGGCGGCTGATGCA |
| 66 | BC3M_380 | TTCACTGTCTGCTGGGGCAGGAGGCAGGGCAGGGGCAGGAGGGAGGCAACCCCAGC |
| | | CTGTGCCCGGCTTCCCCGAGGCGTGTGCCTTGTGCGGCTGCTGAAGGAGTGACTCC |
| | | TGAGGAAACCAGCTTTTCCAGGGAGGCAAGGGATGGGAGAAGAGGGTGGAGAAGGA |
| | | AGTGGTCACACCACTTGCCTTCTGCCAATACTGTCCCTTTCTTACGCGTTAACCTT |
| | | CCACTCTGAGCTATGACACTTTCAGTACTAGTGTGGTAAGTTCTACAGGAAACAGG |
| | | AAACATGGTTTAACAGACAT |
| 67 | BC3M_39 | GCGGGGCTCACGAGTGACGAAGGGCAGAAGGGCGGGGCGGGACgagaggaggggag |
| | | gggcgagcggaggggagggacgagaggaggggcgggacgagaggggggcgggacga |
| | | gaggaggggcggggCTCACGAGTGACGCAGGGCAGAAGGGCGGGGCGCAAGAGAGA |
| | | CTGAGAGCACTACgcgggtgagaggaggggcggggcgtgggagtgacggggcgtgg |
| | | gagtgactgggcgcggagaggccggagccggaggcgaggcgaggcgTGAGAGTGAA |
| | | TGAGGGAGGAGGGCTGTGAG |
| 68 | BC3M_393 | agcaggcacttctgagcctgcagaggaaaggggacttcccggggcccccgagagca |
| | | cagggatgcccggtttgggagccttggctaggcagctgcagctgcgcaggagggtg |
| | | gggcttccgccccgccgactcagaagcgggcggggcttcggcctcttcccggctcc |
| | | cgccagctccgtggagcctggagccccagccgcgcctccctggctgcagctgctgt |
| | | attcacagcagccgcttcaggcgggccgccacggcgatcagtttttcatggcctcc |
| | | aggttctgatgaagcgtggg |
| 69 | BC3M_402 | TAGCATCAGGGTACCTGCTCTGGGCTTGGCTCCTCTTGGCCTTGGCTCCTCTGGGG |
| | | CATCATGGGAACAAGGAGGAGCAGACACCTCGCCAGCCGGGGTGTGTCTGAGCCCC |
| | | AGGAATCCTGCCTCGCAGGGAGGATTCTCTGAGTAGAGGTGATGTGTTATCACAGT |
| | | ATCAGCATTTCTCAGCCTGACTCATGGAGGGGAGTGACTTTACTGTTAGGGCCTGA |
| | | GGGGAAATAATGAGGAACTTCTAGACCAGTTTCATTTTTATTTTTAAACCCACAGT |
| | | TCACCCTTGGGCCTTTTGCC |
| 70 | BC3M_406 | acaagctctgacacagcgtatactcagtaaacatggagtgaatcagttcattcaat |
| | | gaatgaaCGAATGAATGAAACGCCAGAGCCCGCCACAGGGGTCCGCTGCCGCTCCA |
| | | CGCCCGGGCCTCTCACCGGCCAATCAACACTGTGACTCGTACGCCCTGCCCCCTGA |
| | | TGCCACGCCCATCACTCGCCCCTCTGGATTCCCTCCGGCTGCGTGGAAATCCCGGA |
| | | GCACTGGATTTCCCAGAGGCGCCTCCGGTAGCAGTGCGCATGCTCCAGCGCCGGTA |
| | | GCTGAGGCATCAATTTCCCG |
| 71 | BC3M_410 | GCGCCTGCGCCGTGGCGGCCGAACTGGCGCTCAACAGACGGGCGGGGCCGAGCGTG |
| | | AGGCGGAGTCTGCGCACTGCTGCTTTGCAAATGAAGGTGGGCGGGGTGGAGCGAGC |
| | | GTGAGAGACGTGCCCCCGACCAATAAGTGCAGAGATCGCTCGGGGGCGGGGACCTG |
| | | CTGCCGCGCTCCAGGCTGCGGGTGGCCAGAAGGCAGCGGGGGCGGGCTCGGCGCGC |
| | | GCGGCTCCGCCCACTCCGGGCCCCTGCTGGGCGGGAAGGCGGCGCCCCGGCCGAGG |
| | | TGGCGGCGGCTCCTCAGGTA |
| 72 | BC3M_414 | acagaagcaatctgacaaagtttttgtgatgtgtgcattcatctcgcagagtggaa |
| | | ccttaatttcgattgagcagttttgaaacactccttttgtagaatctgtaagtgga |
| | | catttggagcgctttgaggcctaaggtgaaaaaggaaatatcttcccataaaaact |
| | | agacagaagcattctcagaaacttgtttacgatgtgtgtactcaactaacagagtt |
| | | gaaactttcttttgatagagcaAAACAGTAAATTGAAGTTTAAAATAATTGTAACA |
| | | ATTGCATCTTATATATCAGG |
| 73 | BC3M_417 | ACCTGAGGACGCTCAGCGCTGGAGCTCCGAGCAGGAGTTAAAGTACCCGCAGTGGA |
| | | GCTGGCCCGCTGCCTTTCCAGACTGCAAGGCCCGCAGTGCACCGCGCGGGTGACGT |
| | | GTAACAGGGGCGGGCGGGACCGCTGGAGAGCCTATGAGCACAGCGCAAGCACCCCG |
| | | AGGGGCCGCCTTCCGGCCCTATTGGTGAATCCGATTAGGGGTGGGACCGAGCCGTG |
| | | GTGATTGGCGGCCGGAGGGATGGCAAAGCTGCCACGCGCACGGGGGTGCAGGCTGC |
| | | GGGACTGCGATCGCTGCCGG |
| 74 | BC3M_47 | CTGCTGAGGCTGCTCCTGCAGCAGGGGCCATCTTGTTGCTCGGcctcctcttcctc |
| | | ctcctcgtcctccGCCGCCCAGTCGCTCGTTGTCCTCGTCCCCTTCCTCTTCCTCA |
| | | GGCTCCGGCCCGCCCCGGAGACTGGGGCGGAGACGAGGGCGAGGATCCTCCCTCAG |
| | | GAGGCGGGGCGGGCGGAGGGGAGGGGCGGGCGCGGGAGCAAAGCTCTGAGTCACCG |
| | | GCCACCAACGCCCGGAGGGAGACCGGCGACGCTCTCCGCCGCGACCGAAAGTCTCA |
| | | CACGCCCTGAGCAGATGAAC |
| 75 | BC3M_48 | CTTCCTGGGAATGAGTGTCTCACAGCAGCCAGAGGTTGAGGCTTTGTCTTAAGGTG |
| | | GAGGTAATAAAAACCTGTTTGTTTTCCCAGAGCAAGACTTGCCTCAGGGCCCCTGC |
| | | TTGTTTGAGACAGGGCATTCAGTTTGCCTGAGTCAGGCTGGGGAGGTTCTTCTAGT |
| | | CTTTGGAATCCTGTTGGGCAGGGTGGCTGCAGGGGATCTGGAAGAGGTAAGGCCTG |
| | | TCCCAGGGGTGGGGGCTGAGGAGGTGGACATGAAGAACTCCCTGGATTAGGACAGT |
| | | GGCCCAGGAGGGGAAAAGAG |
| 76 | BC3M_49 | AAGTTGGGCAGGGCAGGGGCTAGTCTGCCTTCTTCTGGGCCCAACCCTCCCGGCCG |
| | | GCACCACAGGCATTACAGGTACTCTGTGCACTCAGGCTGCGCAGACCCGCAGCTTC |
| | | CTATCCTGTAGCTCACTTTCCTCTGAGGCGGGCTGGAGGCGGAGCTTGTCCGCTGG |
| | | GGGTGGGGCTCAAAGCTGGGGCGGGGATACGGAGCAAAACTTAAGAGGAAGATGAG |
| | | AAGCCTGGTTGGCCAGGAGGCTTATCTGTCAGGACAGGGGGCGGGGCCTGGGGGGC |
| | | CGTACCTTTGCTTACCGCGA |
| 77 | BC3M_52 | TCATTtttattattagaatctactatttgccaggtactctgaggcaccaggaatat |
| | | acaaataacaagtgcagaaactgaccagtctagttggacaggcagacgcataaatc |
| | | agcaatcacaaggcagtgtgactaatagaggaggtatggcagcacagagagaagtg |
| | | agcagttactcagcctgccttgtaggcagggcactcagagaagcttctcagaggtg |
| | | gtgacatgagagagagctgagccAGTGATACAGAAGCATGTAGCAAGAGTGGGGGT |
| | | ACACTGGCCTGGCAGTGTGA |
| 78 | BC3M_55 | ACCCACGTCCCTCAATCCCCACGAGCAGCTGACTGGGACCTGAAAGTGCCACCAGA |
| | | CGCCCTCACAAGTCTGCTTTCTTTGCTGGGAAACAGCAGCCGCGCCGCAGCCTCCG |
| | | CCCGCTCTGGGGAAGCCCCACCTTGGCAACAAGCCGCTGATTGGCTGGCTCGGGGG |
| | | CGGCGCGGGCCAATCCAAGCCCGCCCTGACGCCGCGGCGTTTGGCCGAGAACTATT |
| | | aagaaaaaaaaaaaaagaaaaaaagaaaGGTGGGGCCGGGCGCTAGGTGGCTTCCC |
| | | AACGGAGTTGCTCCCCCGGC |
| 79 | BC3M_58 | CAAGAGTGGAAAACCTGCCCTCACAGGCCCAGCTGGCCAGAGGGCTTGTCTCTTTC |
| | | AGTCGCCCTCCCCCAGAGGGAGCAGGAGCAGACAATGGCCACCATGACTCACCAGT |
| | | GAGCCATCTTCCCCTCCCCACCCCTCCAGCCTGGCCCATGACAGCTTAGCTTGTCC |
| | | TCCAAGGGAGCTGCAGCCCAGCCTCCCAGGGCCGCCAGCTTCCTCTCTCTTCACCC |
| | | AACCTGGCTCCCCCCCTGCTTGTGCAACACCACATCAGAGGGTTGTGAAGTGGAGA |
| | | GGGAGGAGTTTGACAGCTGC |
| 80 | BC3M_61 | GGGGCTAGCAGGAGAGCCAGAATAAGCAGATTTGGCTTCTAATCTGACTCACCCAA |
| | | CTGGTTCAGAATGCAGCCAAACCGGGGAAATTTGGGTGAGCTCCTCCTCTTCCCCT |
| | | CCCTCACTTGCTCTCGCAGTTGTCCTCTAGCACCTCTCTCTATCCCTCCCTCCCCG |
| | | TCCCCCCGCCCCACTCCCCCAGCTCTGGGAGCGCATGCGGGGGCGGGGTCCTAGGA |
| | | GGATGTGAGCCCATGGACACGCGGGCGGGATGTTTTTCTCCTCGTCATTGTTCTCC |
| | | CATGCCCATTGTGTGCGCTG |
| 81 | BC3M_66 | AGCCACTCACTGCAGAAGGGGCTGGTGAGAGACATGCTCGTCATCTCCGAGGGCCT |
| | | GGCTCTGCGCCAGCCACACACTTATCTGCCTGCTCCATCTCCGGAGTTTCTGTCTC |
| | | TGAGCTTTGGCAATGGAAGTTGTGCTTCCACTATTAGCCAACACCGAGCTGGACTC |
| | | TGGTAACTGACACAGCCGTGCATCTAGTGTAGCTCGGGTTGAGATGACTTGGCttt |
| | | tttttttttttttttttttttgagacggagtctcgctccgtcacccaggctggagt |
| | | gcagtggcgggatctcggct |
| 82 | BC3M_67 | CTATTGTTTGGGCTTTGCTTTTGACTTCACATCCTGAAATAAATGGTCGTTGCAGA |
| | | CCAGGCACGTGAGCAGGAAGTGGGCAGGGCTTAAAACACAGAGAAGTCATAACCTC |
| | | TGCGGTTTGGTTCATGTTGTAATATGAAAACCAGGAAGCTTATCTTGCAGGAGGCT |
| | | GATGTGTAAAAGTTCAGAATGGAGTGGAGCCCTCCCTCTTGGCACCCTATGCGCGG |
| | | AGTCACCCTTTGTCTGCCACAGGAAGCACCCAGGTCCTGGCAGCTAGAAAACTGTA |
| | | ACAACTTGGAAACATTTCCC |
| 83 | BC3M_69 | ACCAGATAAGCACCCACTGCACTCAAGGCCTCTCTGATCAAGTCCCACGACCAGGC |
| | | TCTCCAAGTCCTGACACCGCGGAGACCCCCAAAAGAGGAGGATGGAGCAGAGGGCA |
| | | AGGCTCTCAGCTCCGCGGACTCACACCCAGCTGCAGAGGCAGGGGGAGCCGCCCTT |
| | | TCTGTGGCCGGGGAAATTGAGGTCACTTCCTGTCTCGCTTCCCTCTCTCTGTGCTG |
| | | GCTGCATCCTTCAGAAGGGGGGTGGGTGGCTGCAGGGCAGCGCCAGGCAAGGCTGC |
| | | GGAGAAGCCGGTGCTCCCTG |
| 84 | BC3M_7 | tctgcctgcAAGCTCCAGGTCTTGCAAAGCCTGAGAACTGGTATGGCAAGGGCAGA |
| | | GTGAGAGCAGGGAAGAAATGGAGTCAAGCTGAACAGAGACTTCCGCATCATGAGGG |
| | | TGGTGGGAGGTGGGGAGGAAGTTCTGAAACCACACACATTTATCATTGTTATTGAG |
| | | TCAGACAGACAGTGCCTGCTGACATGTAACTGTCAGGCGTTGCCAAGGCACAGTAG |
| | | GGTTGCAAAGGCTGAGTGTCCACTTCCTCCCAATGAGTCAGGAAGAACCCTTGGAT |
| | | AATTCTCCAAAATAGTTTCA |
| 85 | BC3M_70 | CAGGCACAGTTCTAAGTAATTGaagtctactgaggtaggtatcaatattattccca |
| | | ttctctagatgacgaaactggtgcatgtagcagttaggaaatatgcccaaaggtac |
| | | actgctcgtaagcggcagagcaggaatatgaatccagccagtctggttccggagtc |
| | | tgcattcttgatcactgcactataccaactttcactttgttgtgagcacctgccta |
| | | tctcagacatcagtcagtaagtcccttgaaggcaagaactgtcctttgatccttat |
| | | tcctgagccctaggcattac |
| 86 | BC3M_71 | GGTGTATGTACTGATGTACTGAATGGGCGACCATTTCCTTCCAGAAAGGCTGGAGT |
| | | CAGCCCTCCGGGATGGCTGTCTCTGTGTGACTGTCTGCACACCACTGCCCTCCACT |
| | | GGACACTGAATCAAAGCTGCCCCAGACCCACGTTGGTGTCAGGACTCCCTCAGGTT |
| | | TCCTTCCCTCCCTATCTGGGACACAACCTCCTGGGCAAACCGGTTTCTTGGTTGGC |
| | | TTCTCTTACCAGGTTTGTTTTACCCTGTCTGCCTTGCATTGAATCCATGAAACTTG |
| | | GGAAGTACAAGAGGAACAAT |
| 87 | BC3M_74 | AGGGCATTTCTTGAGCCTGGCAGGAGGCCAGGGGTTTTACAGGGCAGGAAGGAACC |
| | | TGGAGGAACCGAGGAGCCACGTTGTTGGTTGGAAAGAAGGGTGGCCAGGTGGGGAG |
| | | GAGTCTGGCAAAGGGTCCCAGACAGCAGGAAGGGCACCTGTGAAGCCGCCCTGCCG |
| | | AGTGTGTGGTAGAGGCGGGGTGAAATGAGCACTGCTCATAAAAGTGACTGTTGTGA |
| | | ttttttatgagatggagtctcgctctgtcgcccaggctggagtgcaggggagcaac |
| | | ctcggctcactgcaacctcc |
| 88 | BC3M_76 | gatcgcggtgaatatcctgcaggtcatgctacgcccacttgctttgaggttgggaa |
| | | agcagcctcttgaccttcagccacttgagcccagcaggtggagctatttgccctca |
| | | ctggagcctgctttctcgctaaggggaaatctgctaaccattacacagatagcagg |
| | | taagtatttggagttgctcatgattttggaatgttgtggaaacaGGTTTCCTCACT |
| | | TTCAATAATGAACCTTATGATTTATTATATGCAATACAAATACCTGCTGCTGTGGC |
| | | CATGATAAAGGTTCCAGGCC |
| 89 | BC3M_80 | CTGAAGGAGTTAAAACAGTCCCCACCCCCACTCCCGATTTCTAGAACCCCACGATA |
| | | AATTGGGTAAATATGTATTCCATTCATTGGTGCATCTGACCTTGGTCTGTGACAGA |
| | | GGAAAGGCGTGTCTTCTCATACTGTTCCCTATGAACAAAAGGCAAGCAAATGAGGG |
| | | TGACTCAGGACTTCTCATGGCCTACACACAACTGAACATTTTTCTGAATGATTCCA |
| | | CGTATACACTTAGGAATCAGGAAGAGAAACATTTTACTCTTCACTAACCAAATAAA |
| | | ACCATCTATAAATCATATGC |
| 90 | BC3M_82 | AAACAAACACTGGGTTTAGGCATTCTGCTCTCCCAGCACCGCATGGCTGAGGGTGG |
| | | AAAAAAATAACATCTGAAACAGGCCGGGCTTTTGATGATACCTCCTTATGACAGAC |
| | | ACATCGAAAACCACCGACGGTGAGTCACCCACATTCTGTGCATACCCTCTCCGAGG |
| | | AGCAGGAAGTGTGGCTATTTTAAACCCTGAGGCAATGAGAAGTTTTCAGATGCGTC |
| | | CTAAGGCGCTCCGGCCAGCGCCCTGCATGCACACGAGGGCCTTCCTCAGTGTGGCC |
| | | CCAGCACATCTGTAGACCTG |
| 91 | BC3M_84 | ATTTTGACTCACAATGTTGAAACCAGATTATAAATGAGTCATCAGTGAATCGACCA |
| | | CAAAGAGCCTTTGCGGAGGTGATTTACAGGAGAGCTCTGATGTCTGCTGTCCCCTG |
| | | CACACGCTTCACAGAGATGCTGTCAGACGCAGAGCTGGTCTGGGGCATCTGTTGCC |
| | | GCGTCAGCTCAAAAGGATGCTGTGTTGTCACCAATGGGATTCCCCAGCCCAGGCGG |
| | | TGTTGCGGTCCCACCCACACAAGGAAGGCGGCCATCACTGAATAATGCTTGTGGTT |
| | | ACATCATCATTGCTGGTTTC |
| 92 | BC3M_86 | gggatttcctctgctttttcaactaaaatcagctctttcccaaaagcctgtgctgc |
| | | ctgttgtgttttctctgtgtgtgttttgaaatggccttgcgcaccctccagactct |
| | | ctgcctccggggcaagtctgccttttccctgtttccactttgcatactgcataact |
| | | tccttctctgccccacatggacacacgccctcttattcatgcatccgcggctcttg |
| | | ctgcattcgctcggcagcaaagccacaggctcccttgtggatgtcccttgtggaga |
| | | tttgtacttttttaccccac |
| 93 | BC3M_87 | GAGCACAGAAGACGACCCAGCTGAGGCTGGCAGGAGAGACGAAGGCCCCGCCAGAT |
| | | CCCGGAAGCCGCGCCCTTCTGTCCGGCTGCACGCCCGATTGGACGGTTCCTACGTC |
| | | AGCGCCCCTGATTGGATAGGGCTCCAGGCCCCGCCCCCTCAGTCCCTGAGTGACGG |
| | | AGGATGTGATCGGACGCTGGGCTGAGGGCGACAAAGTGACAGGTTCTTGGCTGCAG |
| | | CCTTTTCATGCAGGGCTTCCTGCTTGCGCTGGGCCTGGCCCAGCCCAGGGGGCATT |
| | | TTCATTTAACCTTTTGTATA |
| 94 | BC3M_9 | AGTTTGGATGTTCTCTGTGGAGAGGGAATAAAACCATTGCCTGTTCCCTGGAGGGA |
| | | ATTGGATGCTGAAGCTTCTACCTTTAACAGGGGCATGGGTGCAGTTCCAGCCTCTG |
| | | CCAGCAGGCTGGGCCCTGTGCCCACTTTTGAAAGACCTTCAGGGCTGTGGGGCATG |
| | | AGATGAGAGAGGGAGGGAAGATAATCTGGCTCACtgccgggcactttatgtgactt |
| | | acctccttaattcccccgggcacagccctgagaggaggttggcagtgtctgcattt |
| | | tacagatggggaacttgagg |
| 95 | BC3M_92 | aaacttcgtctcaaaaacaaaacaaaacaaagcgaaaaaacaaaAAAAGTTTCATT |
| | | GTTTCACCTCCACACAGCTCTGTCTGCATTTTGAGCAATGGCCACCAGAGGGCAGG |
| | | AAGAACCAATCTATAAAGCACACAAGGGTTTCACCAACTTTGAAGTCCTCCGTTAG |
| | | AAGGCAAGTTGTCCACTAATATGTAGGAACGATTAATGGCCACCAGAGGGCAGGAA |
| | | GAACCAATCTATAAAGCGCACAAGGGTTTCACCAACTTTGAAGTCCTCCGTTAGAA |
| | | GGCAAGTTGTCCACTAATAT |
| 96 | BC3M_96 | GAAGCagccagaagacctggttctcccaagcctgctacttgctggccatgtaacct |
| | | tgagcaagttatttcctcctctgcaaaaggaagacaataccctcctgcctacttca |
| | | ctcagacgttctgaagatcgatgtagcaatgtggtgtagacatgcttttgtaaCGT |
| | | GGACACACCCAGACAGGAATAAGTCTTGTCCAGGGAATATTTTTTGACAAACACTG |
| | | CTTAACTGGTTTGTCCTCTGAGTGTCACAACTTTTGGCAGAACTTGGTAGTTGGAG |
| | | GTCAGTGGTTGGCTGGTTCA |
| 97 | BC3M_23 | CAGCCCTTCCTCACCTCATCACTCCCCATCCCCCCAAGATATAGAAAGGCCGTGAC |
| | | AGCTGCCAGCCCTGCACATGCTCTTGTTTCAACAGCGGCGATTGCACATCACGTAG |
| | | TCCCCACGTGACCTGTCGGGCCTAGGGCAAGCGCAAAGCTTTCGGAAACCCGAATT |
| | | ATTGCAACCTTGACTTCCTGCCTGTCTCTGAGGCTCCCGGGctgtgctttaagctg |
| | | gacaggcacctgctttacagggaaaaggaccaaggtccggagaggaaaggggcttg |
| | | tcccaggatacgcagcaagt |
| 98 | BC3M_103 | CTAGGAGCTCTGTGCGGAACCGCGTCCAGCCGCCGACTCACTGACACATCACAATG |
| | | AGTCACGTGCTCTGTGCACCGGGCGGATTTGTCAGATCCGCTGCTGCATCACGGCT |
| | | CGGCAGGGCTCTCTGGGTTCTCAGTGCCCTCCTAGGTCTGCAATGCAGTGCGGGAG |
| | | AGGAGGAATATGGGCTTGTGGGGGCAGGGGCAGCGCCCGGACTCCTCCCGGGGCAG |
| | | GACTCCCAGAAACGCAGGAAGCGATGACGCTGCTCAGATAAACCCTGGCGCTCTGC |
| | | GCTGGCGTCCTGGTCAGGAG |
| 99 | BC3M_44 | CATGTGAGCTCAATTAATACAACATATGGTTACTGTACGCCCAAAGGCAACGCATT |
| | | CAAATTGCTTTGTACCATGTAAAACACACACTCTTGAAAAACAGACGCCTAGTGCG |
| | | GAATCCTGTGCACGCCTTTAACTCCTCCAAACGAGCAGGGGGCGTCATGGATTAGC |
| | | ATGTCCCGGGGTTCGGGAATCAGCATTTCCGAGGAAAGGGGCGCTCAGGAGATATC |
| | | CCCACCCCCGATGAGGGGCACTGTCGTGGATGAGTTTAAACCACGCCATAGGCAGC |
| | | CAAGAACTGAGCTCCCGATG |
| 100 | BC3M_219 | GGGACCAATCCAGAAGCAGCACCCAGACCGGTTTACCCGGTTCCAGGACCTTGGGC |
| | | GAAGTCCACCCGCCCGAGGGCAGGGACGACGCAGGCCACGCCGCGGCCCAGTTGCT |
| | | AGCCAGGCAGGGTGGGGATTTGATCTTGCCAAGGAAATGTGAGCGGGAGGCCGAGC |
| | | GTTGGAGGTGGGTAAGTCGTCACTATGCAGGGCGGAGCCATCCTGTGTCTATCACG |
| | | CCCAAGGGCGGTGCATGCAAATTGACTCCCGCATTTGGCTTTTCCCCGGGCTCCGT |
| | | CTCCGCGCGCTGCAACCCGC |

### Example 4: Verification of Carcinoma-Specific Open Chromatin

For verification of the open chromatin regions specific to breast cancer, the nucleic acid fragment obtained by the method described in Example 1 was amplified using the primers shown in Table 3 below.

TABLE 3
Primer sequences for nucleicacid amplification

| SEQ ID NO | Name | Sequence |
|---|---|---|
| 101 | BC3M_102F | GGGGCTCTCAAGGACTCTAC |
| 102 | BC3M_102R | CGAGGGCAGAAAGGAGAGAC |
| 103 | BC3M_11F | GTTTCCGTACGCAGCCTG |
| 104 | BC3M_11R | CAATGAGCAGGAAGATGGGC |
| 105 | BC3M_117F | AAGGCTCAGTGTGTGTATGC |
| 106 | BC3M_117R | GGTTACTGACTGCTCCCCAT |
| 107 | BC3M_119F | TAACCTTCCCTTGGCTTCCA |
| 108 | BC3M_119R | GAGAGAAGGAAAGGGAGGGG |
| 109 | BC3M_125F | ACCTCTAGACCAAGTGCCTG |
| 110 | BC3M_125R | GGTGGCTTCAGAGATGGAGT |
| 111 | BC3M_132F | CTGACGGCAAATTCCTCCAG |
| 112 | BC3M_132R | GCTTGTCTGTCATCTGAGGC |
| 113 | BC3M_137F | GACCAGCCAATCTCCCGG |
| 114 | BC3M_137R | GAGATCCATTGGTTGCGGC |
| 115 | BC3M_139F | GTGTGAGCCAAGTGTTGACC |
| 116 | BC3M_139R | TTCATCCTGCCTGCCTAGAG |
| 117 | BC3M_142F | ctttccactcacaccttgcc |
| 118 | BC3M_142R | aggcacaaaagaggcaaagg |
| 119 | BC3M_146F | GGATGAGTCACTGGATCCGT |
| 120 | BC3M_146R | GCCTCTGTCCCTTCTCCATT |
| 121 | BC3M_154F | AATCCAGTCCCAGTTCCCAG |
| 122 | BC3M_154R | ACTGGCCTCTCAACACCTAC |
| 123 | BC3M_168F | CCCAGAGCTGCAATGTGTAC |
| 124 | BC3M_168R | TACGGATGAGGAGGCTGGTA |
| 125 | BC3M_171F | CCTGTACCTCTGCAGTGCTA |
| 126 | BC3M_171R | TCCTGGGCAGAGTGTTTTCA |
| 127 | BC3M_172F | CCTCCTTCCCATttctgcct |
| 128 | BC3M_172R | ccttttccatttccagcccc |
| 129 | BC3M_173F | GAATCCGCAGATCCTCAAGC |
| 130 | BC3M_173R | AAGTTCTTCTTCCCGCCCTC |
| 131 | BC3M_178F | ACCATTCAGTGTAAGCCCCA |
| 132 | BC3M_178R | TCTTTCCACCATGCACGTTG |
| 133 | BC3M_179F | ACACCACCACCTCCTTCTTC |
| 134 | BC3M_179R | GGAAAGTGCAAATGAACCCCA |
| 135 | BC3M_182F | tggttccctcctcacatcag |
| 136 | BC3M_182R | TTGCAACCTCCGCTTGAAAA |
| 137 | BC3M_199F | TTGGGAAGGCAAGAGGATGA |
| 138 | BC3M_199R | GTGTTCAAGCCCTCCCTCTA |
| 139 | BC3M_2OF | CTCACACCGTCTCACTAGGG |
| 140 | BC3M_2OR | GAATTCCACAGACACCGCG |
| 141 | BC3M_203F | CCTCGTAGGGCTTGAAATGT |
| 142 | BC3M_203R | AGAAGAATTTGGCGAGGATTACC |
| 143 | BC3M_206F | TTGAGCGCAGTCTGGAAATG |
| 144 | BC3M_206R | GAGATGAGCCCTGTCACAGT |
| 145 | BC3M_212F | AAGTCCTAGAGCACCGGAAC |
| 146 | BC3M_212R | CTTTTCTCCGCAGCGATACC |
| 147 | BC3M_22F | ggaaaatcccttgaagccgg |
| 148 | BC3M_22R | TGGGCTGTTGTAATGGTGTG |
| 149 | BC3M_221F | tgatgagctactacgcctgg |
| 150 | BC3M_221R | TGGGAAGTTGAGGGATGTGT |
| 151 | BC3M_224F | CGGGGAGGAGATGAGCTAAA |
| 152 | BC3M_224R | AGTACGTCGAACAGGGGATC |
| 153 | BC3M_226F | TGGATTGATACGGGGCTCTT |
| 154 | BC3M_226R | CGACAGCTGGTTTCACAAGT |
| 155 | BC3M_230F | CTGAGAGGCCCGCAATGT |
| 156 | BC3M_230R | CACTCAGATCTCGCCGCG |
| 157 | BC3M_231F | GAGCGGTGCAAAGGTTCTTA |
| 158 | BC3M_231R | CCTACCTCGTGCTCTTGGAA |
| 159 | BC3M_232F | CTCTCCAAGCACCACTCCC |
| 160 | BC3M_232R | TGTGGAGACTGAACCTTGCA |
| 161 | BC3M_235F | CAGTCCCTACACCCCACAAT |
| 162 | BC3M_235R | CATTTCCCAGAAGACCACGC |
| 163 | BC3M_239F | TGGTTAAGGTGTAGGGGTGG |
| 164 | BC3M_239R | AGCACAGAGTTACCACCTCC |
| 165 | BC3M_241F | GTGTAAACAAACCCAGGCCC |
| 166 | BC3M_241R | GACTCCATCACCGTCCCAA |
| 167 | BC3M_245F | GCGCGCTTTTAAGGAGAGTT |
| 168 | BC3M_245R | GGCTCTGACGGCATTCATTC |
| 169 | BC3M_247F | GATGCAGATGTCAAAACGCG |
| 170 | BC3M_247R | gagggtctcgcttgtttgc |
| 171 | BC3M_250F | AGGAGTTCAGCATAGCACGA |
| 172 | BC3M_250R | CAAGCTGTGCCATAACCCAA |
| 173 | BC3M_252F | CTGTCCTCCTCCCTCCCTC |
| 174 | BC3M_252R | TTTCTTGTCCACACTCCTGG |
| 175 | BC3M_253F | ACACCCGACAGAGTCCAATG |
| 176 | BC3M_253R | GGTAGTCACTCCTTTGGCCT |
| 177 | BC3M_255F | CGGCTCTGAGTCTGAAGCTA |
| 178 | BC3M_255R | GGAGCCTCTGTACCTGTGAC |
| 179 | BC3M_257F | CCCCACAGTGGTCACGAG |
| 180 | BC3M_257R | ggggaaggagaacagagagg |
| 181 | BC3M_260F | gctcaaatgatccgctctcc |
| 182 | BC3M_260R | gactctgatgctgccatgtg |
| 183 | BC3M_265F | GCGGAAGTCATGTCTGGAAC |
| 184 | BC3M_265R | CGAGGCCAGGAAACACTAGT |
| 185 | BC3M_266F | GGCACAAAGGAAACGAAGGA |
| 186 | BC3M_266R | CCAAGCACCTCAAGCCAAAT |
| 187 | BC3M_267F | CCTCCCTGGTAGAATCTGGC |
| 188 | BC3M_267R | GTGTCTCAGGCTCAGTTCCT |
| 189 | BC3M_268F | CTGTGCTGTTCCGTGTGC |
| 190 | BC3M_268R | GAGACCTTTTCACGGGATCT |
| 191 | BC3M_269F | acctctgtgtgaagtgctct |
| 192 | BC3M_269R | gcgagtcaaaggtgtggttt |
| 193 | BC3M_27F | TCTAAGACTGGCTGCTCTGC |
| 194 | BC3M_27R | AAAACGCCCTTTCTGCTCAG |
| 195 | BC3M_275F | GGGTGGGTCTACTTCTGAGG |
| 196 | BC3M_275R | CTCCCTTTCGGCTTCATGTG |
| 197 | BC3M_277F | tggaatggaatcaacccgag |
| 198 | BC3M_277R | cgaaaccgttccattccagt |
| 199 | BC3M_283F | aacaggggagttctcatgcc |
| 200 | BC3M_283R | agccacatcagagacagagc |
| 201 | BC3M_284F | GTTCAAATGTCAGGCCTGCT |
| 202 | BC3M_284R | CACCTCCAAAGACAAACGCA |
| 203 | BC3M_290F | gcccacgtgactagcatagg |
| 204 | BC3M_290R | GAGCGAGAACTGGGAGTGC |
| 205 | BC3M_291F | ATCACCCTGAGCCTTGGAAG |
| 206 | BC3M_291R | CAGGTAATGCAGCGGTTCAT |
| 207 | BC3M_292F | CAAGGGACCCAGAGATCACA |
| 208 | BC3M_292R | ACAGCAAACACAAAAGCCCA |
| 209 | BC3M_295F | TCTGCGAAAGAGGAGGTGAC |
| 210 | BC3M_295R | CATTCCAGAACCACAGGCTG |
| 211 | BC3M_307F | AGCCTCCGTCAGTGTCTTC |
| 212 | BC3M_307R | TGAGACTCTAGCCCTTCCCT |
| 213 | BC3M_321F | ctctccataagacacgccca |
| 214 | BC3M_321R | aagaggcgggtcattcagaa |
| 215 | BC3M_323F | caaagtgccgggattacagg |
| 216 | BC3M_323R | TCCCAAAGAGTGTCACAGCA |
| 217 | BC3M_326F | CCCTCTCCTCTTGCATGACT |
| 218 | BC3M_326R | ATGCCTCTTTGCTGTTCTGC |
| 219 | BC3M_334F | TCGCCACTCTCAGTCAAACT |
| 220 | BC3M_334R | CCCTAGGGCAAAATCAACTGT |
| 221 | BC3M_353F | tgttgtagcctgagtcggtt |
| 222 | BC3M_353R | CCCCACTTGCTTCTGTAGGA |
| 223 | BC3M_360F | GGGAAGGAAGGTTGGTGAGA |
| 224 | BC3M_360R | ACCGTATAGagcagagtggc |
| 225 | BC3M_362F | TCCAAGTCTAAGGGTGCTGG |
| 226 | BC3M_362R | TACCATCCTCTGCTTTGCCA |
| 227 | BC3M_367F | GAGGGCTTTCAGAACTCAGC |
| 228 | BC3M_367R | gcaacgcctTCCTGTTAAGA |
| 229 | BC3M_37F | TTCCCTCGACCTCCCTTCTA |
| 230 | BC3M_37R | CCCTGTCCTGCCAGCTATAG |
| 231 | BC3M_380F | GACTCCTGAGGAAACCAGCT |
| 232 | BC3M_380R | CAGAGTGGAAGGTTAACGCG |
| 233 | BC3M_39F | GCAAGAGAGACTGAGAGCAC |
| 234 | BC3M_39R | CCCTCCTCCCTCATTCACTC |
| 235 | BC3M_393F | gctgcagctgctgtattcac |
| 236 | BC3M_393R | aggggtacagggcagaaaat |
| 237 | BC3M_402F | GGAACAAGGAGGAGCAGACA |
| 238 | BC3M_402R | CTCCATGAGTCAGGCTGAGA |
| 239 | BC3M_406F | GGCCAATCAACACTGTGACT |
| 240 | BC3M_406R | TCCAGTGCTCCGGGATTTC |
| 241 | BC3M_410F | CCGAACTGGCGCTCAACA |
| 242 | BC3M_410R | CTCTGCACTTATTGGTCGGG |
| 243 | BC3M_414F | tgtgtgcattcatctcgca |
| 244 | BC3M_414R | cctcaaagcgctccaaatgt |
| 245 | BC3M_417F | GCAGGAGTTAAAGTACCCGC |
| 246 | BC3M_417R | GCTGTGCTCATAGGCTCTCC |
| 247 | BC3M_47F | CTTCCTCTTCCTCAGGCTCC |
| 248 | BC3M_47R | CGGTGACTCAGAGCTTTGC |
| 249 | BC3M_48F | GGCTGGGGAGGTTCTTCTAG |
| 250 | BC3M_48R | TTCATGTCCACCTCCTCAGC |
| 251 | BC3M_49F | CCGCAGCTTCCTATCCTGTA |
| 252 | BC3M_49R | ACCAGGCTTCTCATCTTCCT |
| 253 | BC3M_52F | atggcagcacagagagaagt |
| 254 | BC3M_52R | Tggctcagctctctctcatg |
| 255 | BC3M_55F | AGCTGACTGGGACCTGAAAG |
| 256 | BC3M_55R | CCCGAGCCAGCCAATCAG |
| 257 | BC3M_58F | CAAGAGTGGAAAACCTGCCC |
| 258 | BC3M_58R | GAGGGGAAGATGGCTCACTG |
| 259 | BC3M_61F | CTCTTCCCCTCCCTCACTTG |
| 260 | BC3M_61R | CATGGGCTCACATCCTCCTA |
| 261 | BC3M_66F | AGCCACACACTTATCTGCCT |
| 262 | BC3M_66R | CCCGAGCTACACTAGATGCA |
| 263 | BC3M_67F | AAGTGGGCAGGGCTTAAAAC |
| 264 | BC3M_67R | GGGCTCCACTCCATTCTGAA |
| 265 | BC3M_69F | AAGAGGAGGATGGAGCAGAG |
| 266 | BC3M_69R | GAGAGAGGGAAGCGAGACAG |
| 267 | BC3M_7F | GGTGGGGAGGAAGTTCTGAA |
| 268 | BC3M_7R | CTTTGCAACCCTACTGTGCC |
| 269 | BC3M_7OF | atgacgaaactggtgcatgt |
| 270 | BC3M_7OR | tcaagaatgcagactccgga |
| 271 | BC3M_71F | CCCTCCACTGGACACTGAAT |
| 272 | BC3M_71R | AGAAGCCAACCAAGAAACCG |
| 273 | BC3M_74F | TTGGAAAGAAGGGTGGCCA |
| 274 | BC3M_74R | CTCATTTCACCCCGCCTCTA |
| 275 | BC3M_76F | tttgaggttgggaaagcagc |
| 276 | BC3M_76R | agcagatttccccttagcga |
| 277 | BC3M_8OF | TGCATCTGACCTTGGTCTGT |
| 278 | BC3M_8OR | GGCCATGAGAAGTCCTGAGT |
| 279 | BC3M_82F | AGACACATCGAAAACCACCG |
| 280 | BC3M_82R | GCCTTAGGACGCATCTGAAA |
| 281 | BC3M_84F | AGGAGAGCTCTGATGTCTGC |
| 282 | BC3M_84R | GCATCCTTTTGAGCTGACGC |
| 283 | BC3M_86F | tgtgctgcctgttgtgtttt |
| 284 | BC3M_86R | atgtggggcagagaaggaag |
| 285 | BC3M_87F | CAGGAGAGACGAAGGCCC |
| 286 | BC3M_87R | TCACATCCTCCGTCACTCAG |
| 287 | BC3M_9F | CTTTAACAGGGGCATGGGTG |
| 288 | BC3M_9R | TCTCTCATCTCATGCCCCAC |
| 289 | BC3M_92F | CAGCTCTGTCTGCATTTTGAG |
| 290 | BC3M_92R | TGGTGGCCATTAATCGTTCC |
| 291 | BC3M_96F | ctggccatgtaaccttgagc |
| 292 | BC3M_96R | TGTGTCCACGttacaaaagca |
| 293 | BC3M_23F | ATAGAAAGGCCGTGACAGCT |
| 294 | BC3M_23R | GCAGGAAGTCAAGGTTGCAA |
| 295 | BC3M_103F | GGGAGAGGAGGAATATGGGC |
| 296 | BC3M_103R | AGGGTTTATCTGAGCAGCGT |
| 297 | BC3M_44F | GGGCGTCATGGATTAGCATG |
| 298 | BC3M_44R | CAGTTCTTGGCTGCCTATGG |
| 299 | BC3M_219F | GACCAATCCAGAAGCAGCAC |
| 300 | BC3M_219R | GCAAGATCAAATCCCCACCC |

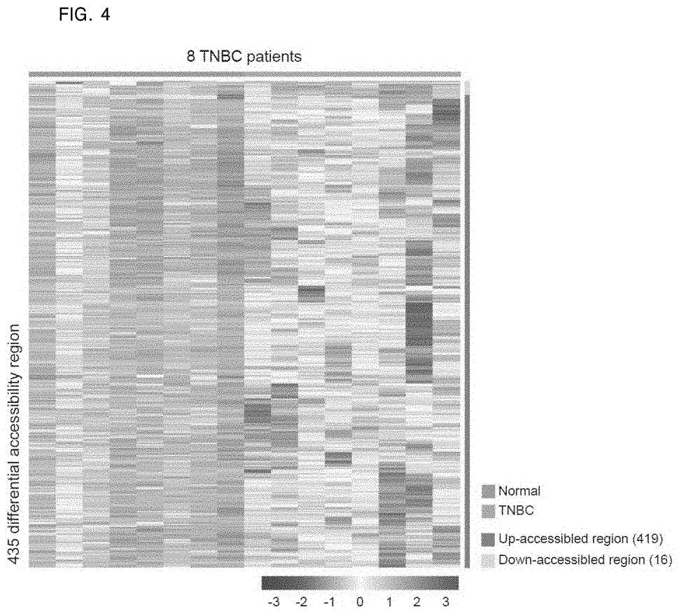

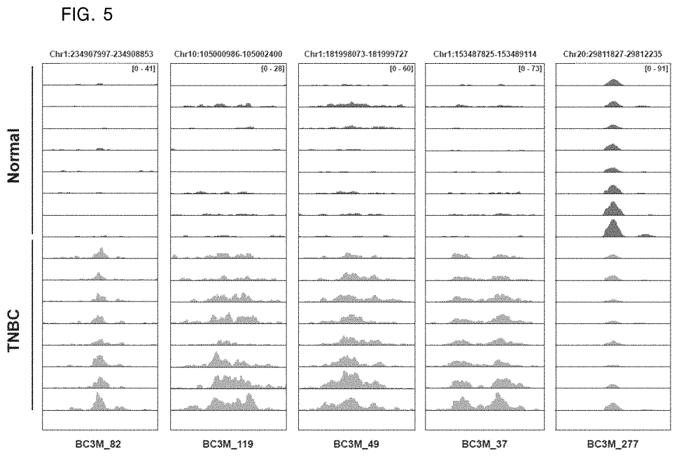

As a result, like the results shown in FIGS. 4 and 5, it was confirmed that an amplification product was detected in the marker sequences having an open chromatin structure in the cancer patients, and no amplification product was detected in the marker sequences having a closed chromatin structure.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The open chromatin structural variation marker according to the present invention is useful as a cancer diagnostic marker because it can confirm the structural variation of chromatin with high accuracy. In addition, the open chromatin structural variation marker may be used as a new cancer diagnostic marker when detecting chromatin structural variation using the composition for detecting the marker.

### SEQUENCE LISTING FREE TEXT

Electronic file is attached.

1. A composition for diagnosing breast cancer containing:
transposase; and
a primer pair specific to any one nucleic acid selected from the group consisting of SEQ ID NOs: 1 to 100.

2. The composition of claim 1, wherein the transposase is Tn5 transposase.

3. The composition of claim 1, wherein the nucleic acid comprises a primer pair specific to each of the nucleic acids represented by SEQ ID NOs: 1 to 20.

4. The composition of claim 3, wherein the nucleic acid comprises a primer pair specific to each of the nucleic acids represented by SEQ ID NOs: 21 to 40.

5. The composition of claim 4, wherein the nucleic acid comprises a primer pair specific to each of the nucleic acids represented by SEQ ID NOs: 41 to 60.

6. The composition of claim 5, wherein the nucleic acid comprises a primer pair specific to each of the nucleic acids represented by SEQ ID NOs: 61 to 80.

7. The composition of claim 6, wherein the nucleic acid comprises a primer pair specific to each of the nucleic acids represented by SEQ ID NOs: 81 to 100.

8. The composition of claim 1, wherein the primer pair is any one or more primer pairs selected from the group consisting of SEQ ID NOs: 101 to 300.

9. The composition of claim 3, wherein the primer pairs are primer pairs represented by SEQ ID NOs: 101 to 140.

10. The composition of claim 4, wherein the primer pairs are primer pairs represented by SEQ ID NOs: 141 to 180.

11. The composition of claim 5, wherein the primer pairs further comprise primer pairs represented by SEQ ID NOs: 181 to 220.

12. The composition of claim 6, wherein the primer pairs are primer pairs represented by SEQ ID NOs: 221 to 260.

13. The composition of claim 7, wherein the primer pairs are primer pairs represented by SEQ ID NOs: 261 to 300.

14. A method for diagnosing breast cancer comprising steps of:
obtaining a nucleic acid fragment by treating a nucleic acid, isolated from a biological sample, with transposase; and
detecting a chromatin structure of the nucleic acid by amplifying the obtained nucleic acid fragment using primer pairs specific to any one or more nucleic acids selected from the group consisting of SEQ ID NOs: 1 to 100.

15. The method of claim 14, wherein a method for detecting the chromatin structure of the nucleic acid comprises detecting the presence of an amplification product.

16. The method of claim 14, wherein the primer pairs are primer pairs represented by SEQ ID NOs: 101 to 140.

17. The method of claim 16, wherein the primer pairs further comprise primer pairs represented by SEQ ID NOs: 141 to 180.

18. The method of claim 17, wherein the primer pairs further comprise primer pairs represented by SEQ ID NOs: 181 to 220.

19. The method of claim 18, wherein the primer pairs further comprise primer pairs represented by SEQ ID NOs: 221 to 260.

20. The method of claim 19, wherein the primer pairs further comprise primer pairs represented by SEQ ID NOs: 261 to 300.


SEQUENCE LISTING (324 sequences)

SEQ ID NO: 1 - DNA, 300 bp, Artificial Sequence
  Synthetic construct
gggatccctcagcagctccggacctcatctgccccacttcggcatcccgcgcgggaatatgaccatgtaggagtaacccggggctctcaaggactctacggtttgtcacggtttgaacgcaagcgcagggcctggggcgggtgcaggtggagggtcggcctctttctgcccttgggaacgcccctttctggatgtggaccggcgaggcggtctctcctttctgccctcgcctggtgaaatgtgggcactgctgccaggagaaaaaaaactgaagctgtgaattcagttcatcacccttcc

SEQ ID NO: 2 - DNA, 300 bp, Artificial Sequence
  Synthetic construct
agcggggctagacggagtcaggggcggaccgccacagcctgcaccaatcaggacccggttgataggcagagcctggcgacttcgaagactcgcccccagtcaaagagccccggggattcgtttccgtacgcagcctggaaaccagcctgggcctatcctgcgcgccgctgcgggctactattggctgccaagaaaccccgcccatcttcctgctcattggccggtgcggtttacgtaagaggagcctgttgctgagcgaaaagtctgttctgcaattttcgctaaggagttgttaacgct

SEQ ID NO: 3 - DNA, 300 bp, Artificial Sequence
  Synthetic construct
gtacactgactttggaacaaatgccacaggccctaattgcaggctccaaggagttgagattccatactggggttgctggaggcagaagccttcccactttcaggacccggacctgcccttcccccacgcggtcccgcccagccagctacaccctggccacagagcgctcacaaaggctcagtgtgtgtatgccgggctgactcacagtggttctgggcccaggcgaggaccttctcagaggggcggaaggggccctctccctcctggccattttccatggggagcagtcagtaaccagga

SEQ ID NO: 4 - DNA, 300 bp, Artificial Sequence
  Synthetic construct
ctaggagacaagtaccctgctgagcagacaaatagcctggactttgtaacagccaaagtggcccacatggcactcgcggggctgtgcagcatccaggcaggggacactgcctggcattctaaaggcctgtgctgagtcatctttcacaggaaccagcttctcaagtctctgggatcctgttttacaggctgttactaaccttcccttggcttccaggccaaggaagaagaaagaataaatattaaccaaaggtacggctgtggcagggtgcccagggcccctccctttccttctctcccc

SEQ ID NO: 5 - DNA, 300 bp, Artificial Sequence
  Synthetic construct
ccacctctagaccaagtgcctgcctggaatgtcctgtccaacttatccaccagctcatccttccgggcctaattaaggccccactccatctctgaagccaccccatgctcatgactctccctgacgcaggttcccgacacaccgggtgactcagctgcagtgtttttcacagtccgtgatgcgtcacagctatttataggtgtgcttaactccctgtgaggaagcacttcaacccccaaacgcaagttccagaaatatgctcataaagataaagatagagaaaagctctggaaaaataca

SEQ ID NO: 6 - DNA, 300 bp, Artificial Sequence
  Synthetic construct
tctggaagcaagttacccacaggtttagtttgcctggagagaaacaggccggagagagactgcggcctccctagggtcttctgacggcaaattcctccagctcagtggctgctgggcagcagcacagccggtttctctcaagggcacaccccacacaccgcgtcactgtgcactagcctcagatgacagacaagcctttcacaagacttttgtggcactgttcatttctgagaccttctctatgatgagctcaaactgcttacctcagagaagaaactgcgtgcacagaaagctgctgag

SEQ ID NO: 7 - DNA, 300 bp, Artificial Sequence
  Synthetic construct
taatttctccgaggccagccagagcaggtttgttggcagcagtacccctccagcagtcacgcgaccagccaatctcccggcggcgctcggggaggcggcgcgctcgggaacgaggggaggtggcggaaccgcgccggggccaccttaaggccgcgctcgccagcctcggcggggcggctcccgccgccgcaaccaatggatctcctcctctgtttaaatagactcgccgtgtcaatcattttcttcttcgtcagcctcccttccaccgccatattgggccactaaaaaaagggggctcgt

SEQ ID NO: 8 - DNA, 300 bp, Artificial Sequence
  Synthetic construct
cctgactgtgaaagccaggccccagcccaagaaggcttcacagacccctaggtgtgccctctgtgtgagccaagtgttgaccctggcgatgatgccaacagcccgactctgcccagctttcagccgcatgagtgtgaaccagctgagcggcaccagctcagggcaaggcagaaggccaggtgcactgtctctaggcaggcaggatgaacagcagcacctgatgtcacagcggccggggaaccaccctggttggggcatgctaacccaccctgctaatatgctttgggtcctaatttcctt

SEQ ID NO: 9 - DNA, 300 bp, Artificial Sequence
  Synthetic construct
ggctcacaccctccaggggctaccctggtcactcagggtaaaagccacagcccttccagtggccttcaaggccctggtgatctgctcgcccctcccctttccactcacaccttgcccccccactcctggcaacccgtctctgctccagccacacacttgcttcattgctgttcctggaaaatactgggcatgttctggcctcggggcctttgcctcttttgtgcctgctgccaggacatctgttcctccggaaagcagcctggatcattcccttctctccttcagggctttattcaaaaa

SEQ ID NO: 10 - DNA, 300 bp, Artificial Sequence
  Synthetic construct
ggatgagtcactggatccgttttcagttcgttccacccacagatccgtcctttgcaggcgccccagaaaagattgcttcagagctggcaccaatggagaagggacagaggcccagcaacagggcgggattggcaggcggaagggagcgtgtgatgagctgagctcacaaagggccggggtgctgggctgcagctggggagggcggggttggatcagcgcctgctcctccgccttcgtttttcccctccccctaaggattcagttcccccttctgaaattcaccaccttgtatgtgactta

SEQ ID NO: 11 - DNA, 300 bp, Artificial Sequence
  Synthetic construct
cacctttcccaagatgacgacatacctaattttgcatagcacctgagattgtaactaaggtggtgggaacccttggtgacttgctgtgttgtgttggccagtgttaacactcacttccccttaacagccctccaaacccaaaaggctatgtcaaatccagtcccagttcccagttccttgtgactgagcccctcaccccgctggacattcctctccaagcaggcagtgcttccttataccctccccacacgggtaggtgttgagaggccagtactgaggtaaatttctttcttatgggca

SEQ ID NO: 12 - DNA, 300 bp, Artificial Sequence
  Synthetic construct
gaactcatgagtcagggtcagtcagcccagagctgcaatgtgtacgtgcttcccggccctgctcttctggcccgcccccaagccttcacgcatgcacccctgcaggcacttaccagcctcctcatccgtatatcctggaaagggtgcaagcatgcctggcttagtcatccatccacaggaagtttgcacagccctacctgagtgctaagatcaggctgtaaactgccagaatgaaacaaaagagggaaaataaatatcagcactctcccataaattttgcaatagtcagctgtagtctag

SEQ ID NO: 13 - DNA, 300 bp, Artificial Sequence
  Synthetic construct
cagacagaggccgctgaattaacccgtggaggcgtctctctgagcagagcccgcaatgcgcctgcttggggctccctgcagcctctgggggaggcagggcggcccagagcaggcctgtgctggaaaggaacgcgaagccctgtaaccaagcctgtacctctgcagtgctagtcccaaggggcctccgagctgtttgtcaccatgtgattggctcaggagaggggtggagaaatgaaaacactctgcccaggatatatttagttgaagtgcagctggggaagtgcttaaacaagggagctt

SEQ ID NO: 14 - DNA, 300 bp, Artificial Sequence
  Synthetic construct
tcttggatttctgaatatgcagttctgttcctaaccagtgtgtcccaaccagaaagtcactgtaatttttggttttgttcccaatctccctccaaatgtcattagtcatatcctccttcccatttctgccttgaataggcagtcattatgatgaagccaggcttgtttcagaattccatgagaaccacagtgtcaggctgtgacaactctggggctggaaatggaaaaggctgtgatctggggttggctggcaccgtccccgtgagtcattatggaaacactgtccccggattctgctga

SEQ ID NO: 15 - DNA, 300 bp, Artificial Sequence
  Synthetic construct
attggtgaggcgccgcgcctcggtgtcgcagcgaatccgcagatcctcaagccaggtgggggcgcccactgcgcgtgtgcagcgcctgatagccaggctagctgagggcggggagcagctgcggcacctgggacacagcgattggctgggaccaggagagggcgggaagaagaacttggcggagcgcgctcatatctctgattggctgccaagggtagcccttgacagctgccgggtgggacccgtagaccgcgagcgcactggcccgtgattggttggggtgcggcggcgagcatctgc

SEQ ID NO: 16 - DNA, 300 bp, Artificial Sequence
  Synthetic construct
agcacttcccgggcgccccgcctcagtttccccatctataaagtggagatgataatagcattcagagtcactgatctaagggctcagggacaccattcagtgtaagccccatacactccctgcaagaggaagctggttctgactcagccttgaggctggcgtctgaggcaaccacaagcccaacgtgcatggtggaaagatgactgtaagtgggggcaacctcagctggccttgggtttgaccatggaatgcgaggcacaaaggggcccattttgcatactttctcagaggctgtagggc

SEQ ID NO: 17 - DNA, 300 bp, Artificial Sequence
  Synthetic construct
cccccgacaccaccacctccttcttcgccttgcatcggtacgataaggcacttgcttgacgggaaagagaaactcagctgccagctggggttcatttgcactttcccccgcctggtctgcggtctggctgtgcagctagccgctctgacggggaggaggggcccaaagccactgcctgccgcctgggcaggggagaggggcacgtgaggctcatggcagaggcacagccagcttcttgcatgtgccctccccggggaatgtctgcagagcccaagactgccacgccgtgggcacagccct

SEQ ID NO: 18 - DNA, 300 bp, Artificial Sequence
  Synthetic construct
gggaaaccttgcagactgtggggtcctgcacacctagacttgctccttttagaagccatggaggaggttgataatgggaataacatttattgtagcttatctctatgccttgagcaatgtgctcacactggctggttccctcctcacatcagcctgatgagtcagatcctgttattacttctcactttacagatgaggaagtagcagtaaatccattacccttttcaagcggaggttgcaagaggttgcaagcggaggcagaataaacacttgaaacagtgagtcagatcctgttatcac

SEQ ID NO: 19 - DNA, 300 bp, Artificial Sequence
  Synthetic construct
ggaaccctaggatctgatttaggacatttggaatctttaaggcacattcgatctagaaagtggaactgaattgctttgggaaggcaagaggatgattttacagtatagggtttgtgtggaaatccccttcagcagtaatcaacccaggtgtccaacctgtttgttaaccatttccaaatgactcagaggacctagagggagggcttgaacacactccagcactgtttctacaatttagcctttatttgcattggaaaccacattcctgaattcttgagggggcaggctctggcttattct

SEQ ID NO: 20 - DNA, 300 bp, Artificial Sequence
  Synthetic construct
tcgtgtgggcctgggccgcttgctattactaataaaacagcagcaaccacaggacagcttcacttccggaaactccctctgtcacgtgctttgcatgaatcctcacaccgtctcactaggggcgctctccccgtttcaccagtgacttggtgacaaccagccttgctcacgaagcgtcagccgtatcctttctgtgtgcagtggggtgtgggttgtgtggagccgcggtgtctgtggaattcacaggctggggccggaatccatggcccccgtcgccgctgccaccccccaggtgctggg

SEQ ID NO: 21 - DNA, 300 bp, Artificial Sequence
  Synthetic construct
aggtggtgcgccggcggttcgcagctgctgtgcccgctggcctgggcgcagccggggacagcgacgcgtttcctgcccgggaagggcccgagcgcagggccggctatagcggtcccgcagctgcctgcttcgattttagcactgctgctccctagagggagcaacgcggccctctgtccctcgtagggcttgaaatgtaaattattcatatcaggggaatgtgtgcttcaaaaagcaagctggacaagaaccgacgggtaatcctcgccaaattcttctatttaaccctcaccattaaaa

SEQ ID NO: 22 - DNA, 300 bp, Artificial Sequence
  Synthetic construct
tgaatgtcatgagtcaggaaaaaagaatttgagcgcagtctggaaatgaaatttcctgcctgtggtttgactcacgtctgtctgtctcgaaatctaccccaaggacatttattccactgtgacagggctcatctctgaggagcaccagactcctgcggtggggagggaagattatccgcgctgcagagactagctggcctccggaagccgcctcctgaccccgcgtcaagcaccgcggtggatggcgcaacccagctttgggaattaattacccaaggcgcgtttccgtgcagtctggcc

SEQ ID NO: 23 - DNA, 300 bp, Artificial Sequence
  Synthetic construct
aatgccctgcccgatccagttccggcctcccatctccccttcccgcgtctccacgctctttccttccccggttctgccgtgaatgctcccaagtcctagagcaccggaactccccgcgcgccttggctcctgggccccagctccgtgcagtcctggactggggctccaggtccaccagggggcgcccgctgcccaagctgggtatcgctgcggagaaaaggggcccagagtgattgttcctcaggggagggagggggaggtccccagagggaagggcctgagtttcctcttgggggatgg

SEQ ID NO: 24 - DNA, 300 bp, Artificial Sequence
  Synthetic construct
aaactaacagggaatggtgttgccacctgtagccccagctacttgagagactgaagcaggaaaatcccttgaagccggcaggcaaagattgctcactacagtctagtctaaaaccccacttccaaaaaaataaaaaacgcacactcacaccattacaacagcccaaaataaatgttcaaacaaaatgttgtctcacacctcgcaacaaacacacaactttctatctgatttttaaacaccgttgatgaaccccaccaacatagggcttcaaaaaatttgcttgaaactcaaaacggtttc

SEQ ID NO: 25 - DNA, 300 bp, Artificial Sequence
  Synthetic construct
cccaaagtactgtgatgagctactacgcctggtcattgtccctctttctcatgactctctggacatccctggggtggagggtggggcaggcacacacatccctcaacttcccagtggttccacgatgactaagccagccctgtccctgaggctgggagtctggagctaggatccacccccatggcctcatatcccaaccttgagcctgggtttctggtcagactggacgggctagctcggtctccttaactctcagagttgccttgtccaggcccagcgggtcccacacagccaggcaca

SEQ ID NO: 26 - DNA, 300 bp, Artificial Sequence
  Synthetic construct
ttacccaagatcattcggtgcggcctcagcgctggcgctgagtcctcttctgccccacccctcaggctcccagtcctggtctagatccctagccacgtagcgtagaagggggcgtcgacgggggttgggctagagttggagcggggaggagatgagctaaagcggggctggctgtgcgagaggcagtagcagcggcgtgtgtcctggggcgccccccggtggcctgtgctggggtcgtcggccgggatcccctgttcgacgtactccggggctgaatgggaaacagacagtcccagaccc

SEQ ID NO: 27 - DNA, 300 bp, Artificial Sequence
  Synthetic construct
aaaaaagactaagtggagatgagggttcagtgcacccccatctcctggccctgctgcccatgagccagaccctgagctgacagattggtgcccatttcctcttatggattgatacggggctcttacctctgggtttgctcagcccagcagcaggcagtcagagccagaagttgtttgcaaaccgaaaccggtctgcggcttgggccacctacttgtgaaaccagctgtcgctgtttttcctccctgtgagaaagtcccccagtaaagctgcgcgggggaggagaaggagggtggaggagg

SEQ ID NO: 28 - DNA, 300 bp, Artificial Sequence
  Synthetic construct
aggggcagggccagggcggttggtggactgggcctggctgtacgtaggtgctctgagaagcccccggcgagaggggcggggccagagcaacagtgggcggggacaggctgtgcgtcggagctccgcggggcctgcggcggggtgggtggggccagggcggcggtgggcgggccgtgctgtgcgtaggggcgctgagaggcccgcaatgtgagaggggcggggccggaacagcggtggacggggtctgtagttcaactgtgccgtggcgtcttcttcgcggcgagatctgagtgcctcgca

SEQ ID NO: 29 - DNA, 300 bp, Artificial Sequence
  Synthetic construct
ggagcggtgcaaaggttcttatcctatttatcggagccagtgtccagaaaaggaagcttgtggtttgagacattctgtaaatccggttccaagagcacgaggtaggactctgaatccgatgtggtttctgttctcggtgatggtgcagagctgtgagccagtggtagggtgtcctttaaattccagctcagtacactagttaatgaacttggctgactgataaaaatgttttcaggtttagctcatgaacatatcaacatagacctaaatataattccagtttgtcatgaatgttgattt

SEQ ID NO: 30 - DNA, 300 bp, Artificial Sequence
  Synthetic construct
ggcctcttgggggcgcggtgagtaggtggcctctccaagcaccactcccgatgtgcgcatgagcgcagccgcccctacgcagcgcgtgcgcacgtgcactcaccacgtccatcccagacgtgcggacccgggtgtctgcaaggttcagtctccacaccccagcgcccgaccctgcgcggggacatgcgcacaagcgcgcgtcctgaccacccggacgtgctggcccacacgcacacgcgtgcgcattacccccgccccatccgcgcctgcgctcaaccccgcctacacctgctccgtggc

SEQ ID NO: 31 - DNA, 300 bp, Artificial Sequence
  Synthetic construct
ggtcctggaccgggacttaggtccacacccacgtgctgacgtcgggcaggctcagcggcctcccgcgcctgcgcagcaccgcccttttcgggcgcggcgcccagtccctacaccccacaatcccccgcgccgttccggaggcgcgctaggagtgggtgtggcctctgcctccacattggaacaaggtgaggcagagggtgtcgcgtggtcttctgggaaatgtagttcgtctgccaggccggaaccaccgctcaaccggctcgcgagactatgcaccccacaatgcgccgcgcgcgcagc

SEQ ID NO: 32 - DNA, 300 bp, Artificial Sequence
  Synthetic construct
tctaagtctgtgcatgcatttgtggtcagagtctggggagctgggggcgtgaatgggctgcttcagacactgctttgagggtgtgaccaggacctgagggtgtggttaaggtgtaggggtggggctaggcccttgggggtgggaccacagtcccagaggcgtggccagggcctcgaaggtatggccatagtttgaggcgtggccgagaaactccgttcccaagggaggtggtaactctgtgctcagagcgccctcttgtggctatcctcaggtctccactttttattcaatagctttatt

SEQ ID NO: 33 - DNA, 300 bp, Artificial Sequence
  Synthetic construct
gtggcccgctgtagccccgccccgtggcccgcccgcagtaggcccgattcaaatctggccaatgatagtgtgtaaacaaacccaggccccgcctcccgacgaataatcccccgaccggcgagaggcccatttaacccgatggggtttggggttgggacggtgatggagtcgtggctccgcccccagacctgggccaataggcggctgggctccgcccccggcacttgccgcgctgaggacccgaggcagggctgggcgcgcagttgcctgatttcgtggcggctcgcagtctgggcgctc

SEQ ID NO: 34 - DNA, 300 bp, Artificial Sequence
  Synthetic construct
cctccaaaagtgctgggattactggcgtgagccaccgcgcccggcctcagggcgcgcttttaaggagagttcctgacatgacggtgggcttttcctgcagatgcacctctgggtagcgccctctttacagccttgaaacctggtcaactacattactcagaaagctctgcgttgaatgaatgccgtcagagccaatgagggctcggaaagaagcatttccgtgtgtgcgcctaatgtagggccgagacttccggggtcctcttgtagcggccacgttgatctgcgatacgcgtgtttgcc

SEQ ID NO: 35 - DNA, 300 bp, Artificial Sequence
  Synthetic construct
acagccttttggaagtcgcgctaaccttggcctgagacctgcaaacttgcccaggctggggcgtgtgaaccggcgagcgcgcagcggaaacggggcggggcacctgaggctgggaatgcagaggagccttccggggggcggggcggggcctcccgtgcagaccaatggtggagtagatgcagatgtcaaaacgcgcgctcaagtggcttccgccaggaatcccgacgcttagggaggcggagggaggatcgcttgagaccagcctgggcaaacaagcgagaccctcgtctgtttacttaa

SEQ ID NO: 36 - DNA, 300 bp, Artificial Sequence
  Synthetic construct
aggctccaaggagttcagcatagcacgagcttttaatttgcgtgcagacaagcacaaaaggcacaaccggatatacctgttatttcccaatgacctgagagcccgaagtttatgttaagccttgggttatggcacagcttgcacgcaaggccctgcagctcctgcaggcaattgagaggtggtggtgtacaggacagaggaacaactctgaagtgacagcacataatttaattccccctaagctttccaagcatgcagactgttccttttttgtcagcgtataacctaagtgatttgttc

SEQ ID NO: 37 - DNA, 300 bp, Artificial Sequence
  Synthetic construct
aacacacacaacacacacacacacactctttcaaggtctagcaaaacccatcaggagaggttgggccctggaggtgctgtggcttcctgctgccccgctccctcccgcctcctccctgcagggctcctcctggggaggcctgtccagctgccaggccccgccccgccacagcccccgctgtcctcctccctccctcagccgtgccagcagcggcacagaactggaattgccctggacggccacagctctgcatatcccccaggagtgtggacaagaaaaaataaacacaattagagttca

SEQ ID NO: 38 - DNA, 300 bp, Artificial Sequence
  Synthetic construct
aagagaagcctgtcagtccagctcgggctacacactgggtgagccatgcaccacccaggaatttccagggcacgtgccacgtaaggggcacacccgacagagtccaatggggttccccactgggcctcccactgagttgctcagcctgggccggaaaagggtgagtcaccctgggggtggggctctccagggtagaggccaaaggagtgactaccatgacaattctccggagggcctgaggcggcggtggacagccccggcaacagtgggccctccccgcagaactgtggttccaatccc

SEQ ID NO: 39 - DNA, 300 bp, Artificial Sequence
  Synthetic construct
gcgtgcttgtgtgtgggtgtgtggtggggtatgtgtgtgtccggggctgccgattcaactgaaaaacaaaagcggctctgagtctgaagctaaggtttaacaagtgaccaagatgactcatgctgcttggctgcaaaggccacagggctgccacccccagcggggcggggcctgggtgggaagagtcacaggtacagaggctcctgtgacattcacactctgcccctgcatcggctgcctttggggccaaatacttttgtgaaaattaagacagaaggccgggtgcggtggttcacgcgt

SEQ ID NO: 40 - DNA, 400 bp, Artificial Sequence
  Synthetic construct
aaacctgcgggccccggtccaggcgtggtcccgctcgcacgagggagcggtcgcccagggtgccgggaagtcggggaccggccagccgccgaccggccgcacccctccccgccgagctcgcgcgcccgcctcgtcagcacctttcccgcagcgcagccccacagtggtcacgaggcgggcgcggcccggtcagccctggctagactaggcatcggcaccacccacctcgcccctccccgtcccgctggtttcccctccccctccttcccctccccctctctgttctccttcccctcccgatccccgggcgggccgcagcgcgccacgtacctggccccgcccctgcgagccacgcagggaaccccggtgacgtcaccaccctccggcgctctcattcccg

SEQ ID NO: 41 - DNA, 300 bp, Artificial Sequence
  Synthetic construct
cctgattagccagaactataggtgcacaccaccacgcctggctaatttttgtatttttttgtagagacagggtttcaacatactgcccaagctggtcttgaactcctgggctcaaatgatccgctctccttggcctgccaaagtgcagggattagaggcgtgagtcaccacgcccagcccattttccttttcctgtccataaattcctctctgaccacatggcagcatcagagtccctctggttcagggagttaccggattcatgaatcattctttgctcaattaaactctgttaacttt

SEQ ID NO: 42 - DNA, 300 bp, Artificial Sequence
  Synthetic construct
tgtttctagctagttataattggcaggcaaccagaagcctcatctgccaagggcggaagtcatgtctggaacaggtttccctcttaagactgtgggctaacccagcatcttgccactttgtgtgggacttcctcattcttagtacataactgtgtttgaccctcagggatgactagtgtttcctggcctcggtacagttgacttctccagaaactatctggctcactctcaatttcctggagccgtatatcctaattacaaaaatgggaaaatcatacctagagtcccatagaaagagaa

SEQ ID NO: 43 - DNA, 300 bp, Artificial Sequence
  Synthetic construct
aaggagagatgatggaggcaacacttacaggtcctgaaaactgctcaaataggcacaaaggaaacgaaggatgcctgaaaataatgatgatgcaaaaactaagctaggtagggcagcaggaagaaccggtttggtgggaagatgatgaatttggcttgaggtgcttggcaagacatgcaagtctgctgcacaggcaatgcaggtcagcaatttgagagaaaggtaaactttcacaatcctaatttgagaagcaacagcacggagatgattatggagccatgagggctgagacactcagcg

SEQ ID NO: 44 - DNA, 300 bp, Artificial Sequence
  Synthetic construct
cccatgcaactgtgtgatgaaacagccccacacatccgggagcacagccaaggcgtcctgtgccacctccctggtagaatctggcttttcaacttgctcacccatgagaggaaagcggttttagacatcaggcttacccctctcctaagccacacccttttctcattcccagctgaggaactgagcctgagacactgaggttcccagctgcctccatgattcgccagcacccagcttcagtttcacatcctcccaatcgtcatagccaggacagcatgcctcactgaccacgagggaatg

SEQ ID NO: 45 - DNA, 400 bp, Artificial Sequence
  Synthetic construct
aggcgccgccgctgagggcaggcagcccggcagccactacacacggacccgtgacgtcgggcgtagcgcggcgcacgtcacggccgctcgctcgtgcgcgcgcacccctccgcccggcggtagcggaacccgccgcgggcgcgcgcccggcccaggggagtgggtcggcgcctgcgcagaggcccgccacgcccacacacaggccaccgcccccaccggccggacggcgcggggattcccagtcctggctccgccccggcctcggccccgcccccgcccctgccccggggcagcctgtgctgttccgtgtgcgcggcgcatacgcacctgggttgtctcgagcctgcggtagtggccagatcccagacatccgagtagatcccgtgaaaaggtctcccac

SEQ ID NO: 46 - DNA, 300 bp, Artificial Sequence
  Synthetic construct
ccagccactgtgagtactggctgctcctgactcacagctgcaccctttgagggagtgaggggcgttacccttggctgacaggatatgattagaaagcctggaaggcggctggtggtggcccatggccaatgagtcactgtgcgagtgtatactagcccagccctcttgcctccaggcaggaaaacctctgtgtgaagtgctctacttgctccatgctctggcgctctctgtacctacgcaggctgaagctgagcctagacatctcctgaaaccacacctttgactcgcttcttccccttc

SEQ ID NO: 47 - DNA, 300 bp, Artificial Sequence
  Synthetic construct
aacttgaaacaaataaagcaggttgaagatcacagtgtgtgctgctgggcctgtgggggcgctgggcagcagaaaggcacactctgcctgcagcctcgggatctggtcgcctgtgtgggagtagggaggagtcctgacgtaccctctctaagactggctgctctgcacctccctccaagccaggctggccagtaaagaaatctagctgtggacaggaaacgagtggtttttgtgatctgagcagaaagggcgttttaggcctggagcagagtggaggccctgagccacggcccaggaagt

SEQ ID NO: 48 - DNA, 300 bp, Artificial Sequence
  Synthetic construct
tttgtgtgcatgtgcgtgtgtgtctgggggaaggaggtagaggaagtgagatgatggtgacagtgacagcagcttggagaagacaggggggtgggtctacttctgaggaagtccttggctgaggtagggccgcagagaggcagggtgagggtggagcctgtggtttcagagaggagttttaatggctgccaagaatgtgcacatgaagccgaaagggagtgcggcctggagctgcagtcagcccagagggcgggtggagcctgtcccagggcactaggatcgcagagaacgacaggaggg

SEQ ID NO: 49 - DNA, 300 bp, Artificial Sequence
  Synthetic construct
aaggtattcgaatcgaatgaaatggaatcgaattgaaggggtatgaatggaatggaatggaatggaatcgaatcgaatttaatggaattgaataggaaagaatcaaatggaatggaatcaacccgagtggaatggaatggaatggaaaggaatggaatggaatggaatggaatggaatggaatggaatggaatggactccagtggaaaagactggaatggaacggtttcgaatgaaattgaatcgaatgaaatggaatggaatgcaatggaatcaaatggaatggacttgaatggaatgg

SEQ ID NO: 50 - DNA, 300 bp, Artificial Sequence
  Synthetic construct
cccatcgtgttgcgaaagcattcaggttgaacagtgttcaggaagaatactcaagcaaaaactggtttgcagccaaatacagagactgcaaaccccagtggcttcaggggccaggcagggaaagtaaacatgtgaaacaatagggagtagtcctgcctgtggggaacaggggagttctcatgccccagcctaataaatgaaaaaattatttatacaccacagtggaaccggagatgcacctaaagccattgggatgtggtttctctttttcatctcactgctctgtctctgatgtggctt

SEQ ID NO: 51 - DNA, 300 bp, Artificial Sequence
  Synthetic construct
agcttggatgctgcacccaggactgaaagggggacctgtgggcggcctctgcctctccccgcgcagcgtcaggacacaggcccacattccctcctggcttctccctgaagggagagagaataatagttggttcaaatgtcaggcctgctccgtgctggtggggagactggttgagcaggtccgcaggagggacggagggaggaaattattaataattgcaaagcaaccagccacactacaggccttgagttgtgtctgcgtttgtctttggaggtgtggagttgggggtgctgatcctgg

SEQ ID NO: 52 - DNA, 300 bp, Artificial Sequence
  Synthetic construct
ctgggggactgttgggtcagaaagtgttcagggagcagctgttgcgccctccctcggccccgccgctcggagacgccccgccccctgccttcaccggccgccccgccccctgccttcaccggccgcccggccacgccccacaccgccccggccccgccccagcgcccacgtgactagcataggcgcgcccctgctccgccccccgccgccgactccgcctccgggacgggagcgagcggcgagcgcgcgcactcccagttctcgctcggcgactcccgcgcacgcgcgcgccgtgccacc

SEQ ID NO: 53 - DNA, 300 bp, Artificial Sequence
  Synthetic construct
ctttttcctttaaagaatacacttcttatgtaatttgttttgcatttctggaatgaggaacttttctgctcatattgttgttaaaatctagacaacacgcccgtgtgatagatcaccctgagccttggaaggaaatgattcaccacaatactgtaactgaaagtcgtctaacaccagggctggaaggcaggctatgaaccgctgcattacctgcgtgcagcagcaatgggaggcagccagaggttccctcggcctgcctagctcacttcagctttgttcctgttctgtttcctccgtccg

SEQ ID NO: 54 - DNA, 300 bp, Artificial Sequence
  Synthetic construct
catcaagggacccagagatcacagaatagccagcccttcattttcaggtgagggcctctgtgggaaggtgcgttccaagccacacagttggaagttgagcgaactgaaccaaggctgggcttttgtgtttgctgtttaaacagtgtgtggttttactcacctaccatagtgctcctcctactggtgggcaccttagagtaggctgaaaacaacgtgtctcactgtccttttttgtttgtctctgagtatttttccttatgatcttgaagtaacatttacttaatttgcaatgaatgaaaa

SEQ ID NO: 55 - DNA, 300 bp, Artificial Sequence
  Synthetic construct
tagcaacatgaggcaaccttgtctgcgaaagaggaggtgaccgcagctcctggggatgtgccaactctgggatgtgacgggaagacaaagggcttctgtccccttctgcctggcggtaagagagccggccgcccggcaggcatgccccagcctgtggttctggaatgcgggcaagccaccgtccccagagacctgtgttggtggccaggccagcccacacacccgattggcacatactcttgtgcttgcccaggagcggagtcagaccattcacgctgccttcatgggagttgaacagtt

SEQ ID NO: 56 - DNA, 300 bp, Artificial Sequence
  Synthetic construct
tgcccccacatcgccatcctgcctgtccttctgggcctgcacgtttgttgtgtttggaaggagccaccaaggaggaggatgtcaatgtgcaagttctcagggaagcaggccccgcagcctccgtcagtgtcttccgtccgcaggaagaacccaggcctgggtgattcatcggggcctcagggccgggaggcactaaatcttctgcagatgtggtaagatcctatcacagcagaaagggaagggctagagtctcagggaaggttttgctagggagacgggcttggagggggctgaggctca

SEQ ID NO: 57 - DNA, 300 bp, Artificial Sequence
  Synthetic construct
caaaaaatactgagcacaaataaatattcactgtaaggcaggaggcagccgggaccagactccagatcagatcgaagactggcggaaactgaggagaggcgcttaaagcccctctccataagacacgcccaccacctccatgacagtttaccattgccgtggcaacacccggaagttactgccccttgccgcggcaacaccggaagttcccgcccactttctagctaattctgaatgacccgcctcttaattagcatgtcttttaaagtggacctaaatacgcctacgaaactgccccta

SEQ ID NO: 58 - DNA, 300 bp, Artificial Sequence
  Synthetic construct
tggtctctatctcctgatcttgtgatacgccggccgcggcctcccaaagtgccgggattacaggcatgagccaccaggcacggctgaacagggtttttttaaagttcctgaactgggtggctgcccacaagagggcactcatgcctctgcgtgtgagtgtggaacctggtcgactgctgtgacactctttgggaagacagtcggcattttccacttccagcagcaggtggcagtatgggcaagagtatcatcacccatctttcatctaccacccatgtgcttacatctgggctgctgaga

SEQ ID NO: 59 - DNA, 300 bp, Artificial Sequence
  Synthetic construct
ctcacccgtaacacacacacacacatgcgcgccctctcctcttgcatgactcctctctcagggctgagctgtttttctgagggtgccacaatgaatcagctgcttagtcatctctggagtgcgggagctagcagaacagcaaagaggcattacaaacccaatagcgggtttcacttccttgagcagtatttattctgctctctacctcatgctgcccaaactgttggagaggccctatccactctccctgccttttcagcccttattctcccaaatgcagccacagaggaggtaagagag

SEQ ID NO: 60 - DNA, 300 bp, Artificial Sequence
  Synthetic construct
tttttgtggttgagttctgaattaaaaagtgtcgtactatatatttgtttggtcatttctatgacttcagcactctcaaagacttggacagaagcataaataagaggcagtgtgagcattctccaagtaatcattccaagttggtgagttcatactccacctagacctcatggcctcgccactctcagtcaaactggtttttgtggttgtcaaagtccaacatggcaaatttcccactgatactaagtgagttgaaaactcaagttacagttgattttgccctagggaattttaccaaga

SEQ ID NO: 61 - DNA, 300 bp, Artificial Sequence
  Synthetic construct
agggcatttgctgagttttgctttatgtgactggatgggactggccttggagacactaataagcacgtgagggtttttggacaatgcgaagagttggtgccaagccacaagtgggagatgttgaacttcctgcgaatctggtgtgttgtagcctgagtcggtttcaatatgaaaaataagagtgacagtgccttccttgtatgctaatctggcgaagtggctcatgctggccatgtaacaacctggcagcctcctacagaagcaagtggggtgtggcattcctgctgtctgcatcttctg

SEQ ID NO: 62 - DNA, 300 bp, Artificial Sequence
  Synthetic construct
tgagtgagctggcaagggaaggaaggttggtgagagtaagtcgtaagtatctttttagaaaaagaaaaaaaaaaaaatagcagaggatggtttcgatccatcgacctctgggttatgggcccagcacgcttccgctgcgccactctgctctatacggtagtgatatttgcagtgaattctttatgatgttttcctcaaaacttggtggggattctggttttttggtatggttaaacaaatctgatttccacaccccaccaagggccactagttctatttatgctgcaaacatgaggatga

SEQ ID NO: 63 - DNA, 300 bp, Artificial Sequence
  Synthetic construct
agacactcgtgccctcaagaacttacaatttaggtttgtttgaaagttaactgagaattccaagtctaagggtgctggtgagagtggcctggcaaagccagcccaggaagagctgctgagcaggttgtaaggaacgaggatgccccaccccccctccttggcaaagcagaggatggtattccagacaggtcacaaacagctcaagcaaagacgtggtgacagggatgaggaaggcacgcttgcggatcgctagaatggaggttgcctgggcacagacaccttggaggatccgattagcaa

SEQ ID NO: 64 - DNA, 300 bp, Artificial Sequence
  Synthetic construct
gcagcacccagttcagaactttgcagattgctggaattgctggggagctgccagagggctttcagaactcagcatgagtgcagtgagtgcggcagccagctcccaaaggggatggcctcagcatagtttccagctctcggctctcttaacaggaaggcgttgcggtgtcgcagacacaatctgaagtgggggttcaaacagacacaacttcacatactggttttgcaacttgctggcaaatgagtgaattttactcaatcccaatttttctcatctgtaaaacagccataaaatcgaccc

SEQ ID NO: 65 - DNA, 300 bp, Artificial Sequence
  Synthetic construct
ccttccctcgacctcccttctaccccttcgccttagatggagattttctctttctgaacccggaaccgctccctcctccccgcccggctatagctggcaggacagggattggatgccacggccggtgcgagccttcgctctccgccgagggtagtgacacaggcgaggacgggccccgcaggtcacatgagggcggggcctggcgggctcgtgaccttcccgtaggcggggtccctcccctcccagctcgggccgacagcgtcgtcaccagcttttatggggcacgtggcggctgatgca

SEQ ID NO: 66 - DNA, 300 bp, Artificial Sequence
  Synthetic construct
ttcactgtctgctggggcaggaggcagggcaggggcaggagggaggcaaccccagcctgtgcccggcttccccgaggcgtgtgccttgtgcggctgctgaaggagtgactcctgaggaaaccagcttttccagggaggcaagggatgggagaagagggtggagaaggaagtggtcacaccacttgccttctgccaatactgtccctttcttacgcgttaaccttccactctgagctatgacactttcagtactagtgtggtaagttctacaggaaacaggaaacatggtttaacagacat

SEQ ID NO: 67 - DNA, 300 bp, Artificial Sequence
  Synthetic construct
gcggggctcacgagtgacgaagggcagaagggcggggcgggacgagaggaggggaggggcgagcggaggggagggacgagaggaggggcgggacgagaggggggcgggacgagaggaggggcggggctcacgagtgacgcagggcagaagggcggggcgcaagagagactgagagcactacgcgggtgagaggaggggcggggcgtgggagtgacggggcgtgggagtgactgggcgcggagaggccggagccggaggcgaggcgaggcgtgagagtgaatgagggaggagggctgtgag

SEQ ID NO: 68 - DNA, 300 bp, Artificial Sequence
  Synthetic construct
agcaggcacttctgagcctgcagaggaaaggggacttcccggggcccccgagagcacagggatgcccggtttgggagccttggctaggcagctgcagctgcgcaggagggtggggcttccgccccgccgactcagaagcgggcggggcttcggcctcttcccggctcccgccagctccgtggagcctggagccccagccgcgcctccctggctgcagctgctgtattcacagcagccgcttcaggcgggccgccacggcgatcagtttttcatggcctccaggttctgatgaagcgtggg

SEQ ID NO: 69 - DNA, 300 bp, Artificial Sequence
  Synthetic construct
tagcatcagggtacctgctctgggcttggctcctcttggccttggctcctctggggcatcatgggaacaaggaggagcagacacctcgccagccggggtgtgtctgagccccaggaatcctgcctcgcagggaggattctctgagtagaggtgatgtgttatcacagtatcagcatttctcagcctgactcatggaggggagtgactttactgttagggcctgaggggaaataatgaggaacttctagaccagtttcatttttatttttaaacccacagttcacccttgggccttttgcc

SEQ ID NO: 70 - DNA, 300 bp, Artificial Sequence
  Synthetic construct
acaagctctgacacagcgtatactcagtaaacatggagtgaatcagttcattcaatgaatgaacgaatgaatgaaacgccagagcccgccacaggggtccgctgccgctccacgcccgggcctctcaccggccaatcaacactgtgactcgtacgccctgccccctgatgccacgcccatcactcgcccctctggattccctccggctgcgtggaaatcccggagcactggatttcccagaggcgcctccggtagcagtgcgcatgctccagcgccggtagctgaggcatcaatttcccg

SEQ ID NO: 71 - DNA, 300 bp, Artificial Sequence
  Synthetic construct
gcgcctgcgccgtggcggccgaactggcgctcaacagacgggcggggccgagcgtgaggcggagtctgcgcactgctgctttgcaaatgaaggtgggcggggtggagcgagcgtgagagacgtgcccccgaccaataagtgcagagatcgctcgggggcggggacctgctgccgcgctccaggctgcgggtggccagaaggcagcgggggcgggctcggcgcgcgcggctccgcccactccgggcccctgctgggcgggaaggcggcgccccggccgaggtggcggcggctcctcaggta

SEQ ID NO: 72 - DNA, 300 bp, Artificial Sequence
  Synthetic construct
acagaagcaatctgacaaagtttttgtgatgtgtgcattcatctcgcagagtggaaccttaatttcgattgagcagttttgaaacactccttttgtagaatctgtaagtggacatttggagcgctttgaggcctaaggtgaaaaaggaaatatcttcccataaaaactagacagaagcattctcagaaacttgtttacgatgtgtgtactcaactaacagagttgaaactttcttttgatagagcaaaacagtaaattgaagtttaaaataattgtaacaattgcatcttatatatcagg

SEQ ID NO: 73 - DNA, 300 bp, Artificial Sequence
  Synthetic construct
acctgaggacgctcagcgctggagctccgagcaggagttaaagtacccgcagtggagctggcccgctgcctttccagactgcaaggcccgcagtgcaccgcgcgggtgacgtgtaacaggggcgggcgggaccgctggagagcctatgagcacagcgcaagcaccccgaggggccgccttccggccctattggtgaatccgattaggggtgggaccgagccgtggtgattggcggccggagggatggcaaagctgccacgcgcacgggggtgcaggctgcgggactgcgatcgctgccgg

SEQ ID NO: 74 - DNA, 300 bp, Artificial Sequence
  Synthetic construct
ctgctgaggctgctcctgcagcaggggccatcttgttgctcggcctcctcttcctcctcctcgtcctccgccgcccagtcgctcgttgtcctcgtccccttcctcttcctcaggctccggcccgccccggagactggggcggagacgagggcgaggatcctccctcaggaggcggggcgggcggaggggaggggcgggcgcgggagcaaagctctgagtcaccggccaccaacgcccggagggagaccggcgacgctctccgccgcgaccgaaagtctcacacgccctgagcagatgaac

SEQ ID NO: 75 - DNA, 300 bp, Artificial Sequence
  Synthetic construct
cttcctgggaatgagtgtctcacagcagccagaggttgaggctttgtcttaaggtggaggtaataaaaacctgtttgttttcccagagcaagacttgcctcagggcccctgcttgtttgagacagggcattcagtttgcctgagtcaggctggggaggttcttctagtctttggaatcctgttgggcagggtggctgcaggggatctggaagaggtaaggcctgtcccaggggtgggggctgaggaggtggacatgaagaactccctggattaggacagtggcccaggaggggaaaagag

SEQ ID NO: 76 - DNA, 300 bp, Artificial Sequence
  Synthetic construct
aagttgggcagggcaggggctagtctgccttcttctgggcccaaccctcccggccggcaccacaggcattacaggtactctgtgcactcaggctgcgcagacccgcagcttcctatcctgtagctcactttcctctgaggcgggctggaggcggagcttgtccgctgggggtggggctcaaagctggggcggggatacggagcaaaacttaagaggaagatgagaagcctggttggccaggaggcttatctgtcaggacagggggcggggcctggggggccgtacctttgcttaccgcga

SEQ ID NO: 77 - DNA, 300 bp, Artificial Sequence
  Synthetic construct
tcatttttattattagaatctactatttgccaggtactctgaggcaccaggaatatacaaataacaagtgcagaaactgaccagtctagttggacaggcagacgcataaatcagcaatcacaaggcagtgtgactaatagaggaggtatggcagcacagagagaagtgagcagttactcagcctgccttgtaggcagggcactcagagaagcttctcagaggtggtgacatgagagagagctgagccagtgatacagaagcatgtagcaagagtgggggtacactggcctggcagtgtga

SEQ ID NO: 78 - DNA, 300 bp, Artificial Sequence
  Synthetic construct
acccacgtccctcaatccccacgagcagctgactgggacctgaaagtgccaccagacgccctcacaagtctgctttctttgctgggaaacagcagccgcgccgcagcctccgcccgctctggggaagccccaccttggcaacaagccgctgattggctggctcgggggcggcgcgggccaatccaagcccgccctgacgccgcggcgtttggccgagaactattaagaaaaaaaaaaaaagaaaaaaagaaaggtggggccgggcgctaggtggcttcccaacggagttgctcccccggc

SEQ ID NO: 79 - DNA, 300 bp, Artificial Sequence
  Synthetic construct
caagagtggaaaacctgccctcacaggcccagctggccagagggcttgtctctttcagtcgccctcccccagagggagcaggagcagacaatggccaccatgactcaccagtgagccatcttcccctccccacccctccagcctggcccatgacagcttagcttgtcctccaagggagctgcagcccagcctcccagggccgccagcttcctctctcttcacccaacctggctccccccctgcttgtgcaacaccacatcagagggttgtgaagtggagagggaggagtttgacagctgc

SEQ ID NO: 80 - DNA, 300 bp, Artificial Sequence
  Synthetic construct
ggggctagcaggagagccagaataagcagatttggcttctaatctgactcacccaactggttcagaatgcagccaaaccggggaaatttgggtgagctcctcctcttcccctccctcacttgctctcgcagttgtcctctagcacctctctctatccctccctccccgtccccccgccccactcccccagctctgggagcgcatgcgggggcggggtcctaggaggatgtgagcccatggacacgcgggcgggatgtttttctcctcgtcattgttctcccatgcccattgtgtgcgctg

SEQ ID NO: 81 - DNA, 300 bp, Artificial Sequence
  Synthetic construct
agccactcactgcagaaggggctggtgagagacatgctcgtcatctccgagggcctggctctgcgccagccacacacttatctgcctgctccatctccggagtttctgtctctgagctttggcaatggaagttgtgcttccactattagccaacaccgagctggactctggtaactgacacagccgtgcatctagtgtagctcgggttgagatgacttggcttttttttttttttttttttttttgagacggagtctcgctccgtcacccaggctggagtgcagtggcgggatctcggct

SEQ ID NO: 82 - DNA, 300 bp, Artificial Sequence
  Synthetic construct
ctattgtttgggctttgcttttgacttcacatcctgaaataaatggtcgttgcagaccaggcacgtgagcaggaagtgggcagggcttaaaacacagagaagtcataacctctgcggtttggttcatgttgtaatatgaaaaccaggaagcttatcttgcaggaggctgatgtgtaaaagttcagaatggagtggagccctccctcttggcaccctatgcgcggagtcaccctttgtctgccacaggaagcacccaggtcctggcagctagaaaactgtaacaacttggaaacatttccc

SEQ ID NO: 83 - DNA, 300 bp, Artificial Sequence
  Synthetic construct
accagataagcacccactgcactcaaggcctctctgatcaagtcccacgaccaggctctccaagtcctgacaccgcggagacccccaaaagaggaggatggagcagagggcaaggctctcagctccgcggactcacacccagctgcagaggcagggggagccgccctttctgtggccggggaaattgaggtcacttcctgtctcgcttccctctctctgtgctggctgcatccttcagaaggggggtgggtggctgcagggcagcgccaggcaaggctgcggagaagccggtgctccctg

SEQ ID NO: 84 - DNA, 300 bp, Artificial Sequence
  Synthetic construct
tctgcctgcaagctccaggtcttgcaaagcctgagaactggtatggcaagggcagagtgagagcagggaagaaatggagtcaagctgaacagagacttccgcatcatgagggtggtgggaggtggggaggaagttctgaaaccacacacatttatcattgttattgagtcagacagacagtgcctgctgacatgtaactgtcaggcgttgccaaggcacagtagggttgcaaaggctgagtgtccacttcctcccaatgagtcaggaagaacccttggataattctccaaaatagtttca

SEQ ID NO: 85 - DNA, 300 bp, Artificial Sequence
  Synthetic construct
caggcacagttctaagtaattgaagtctactgaggtaggtatcaatattattcccattctctagatgacgaaactggtgcatgtagcagttaggaaatatgcccaaaggtacactgctcgtaagcggcagagcaggaatatgaatccagccagtctggttccggagtctgcattcttgatcactgcactataccaactttcactttgttgtgagcacctgcctatctcagacatcagtcagtaagtcccttgaaggcaagaactgtcctttgatccttattcctgagccctaggcattac

SEQ ID NO: 86 - DNA, 300 bp, Artificial Sequence
  Synthetic construct
ggtgtatgtactgatgtactgaatgggcgaccatttccttccagaaaggctggagtcagccctccgggatggctgtctctgtgtgactgtctgcacaccactgccctccactggacactgaatcaaagctgccccagacccacgttggtgtcaggactccctcaggtttccttccctccctatctgggacacaacctcctgggcaaaccggtttcttggttggcttctcttaccaggtttgttttaccctgtctgccttgcattgaatccatgaaacttgggaagtacaagaggaacaat

SEQ ID NO: 87 - DNA, 300 bp, Artificial Sequence
  Synthetic construct
agggcatttcttgagcctggcaggaggccaggggttttacagggcaggaaggaacctggaggaaccgaggagccacgttgttggttggaaagaagggtggccaggtggggaggagtctggcaaagggtcccagacagcaggaagggcacctgtgaagccgccctgccgagtgtgtggtagaggcggggtgaaatgagcactgctcataaaagtgactgttgtgattttttatgagatggagtctcgctctgtcgcccaggctggagtgcaggggagcaacctcggctcactgcaacctcc

SEQ ID NO: 88 - DNA, 300 bp, Artificial Sequence
  Synthetic construct
gatcgcggtgaatatcctgcaggtcatgctacgcccacttgctttgaggttgggaaagcagcctcttgaccttcagccacttgagcccagcaggtggagctatttgccctcactggagcctgctttctcgctaaggggaaatctgctaaccattacacagatagcaggtaagtatttggagttgctcatgattttggaatgttgtggaaacaggtttcctcactttcaataatgaaccttatgatttattatatgcaatacaaatacctgctgctgtggccatgataaaggttccaggcc

SEQ ID NO: 89 - DNA, 300 bp, Artificial Sequence
  Synthetic construct
ctgaaggagttaaaacagtccccacccccactcccgatttctagaaccccacgataaattgggtaaatatgtattccattcattggtgcatctgaccttggtctgtgacagaggaaaggcgtgtcttctcatactgttccctatgaacaaaaggcaagcaaatgagggtgactcaggacttctcatggcctacacacaactgaacatttttctgaatgattccacgtatacacttaggaatcaggaagagaaacattttactcttcactaaccaaataaaaccatctataaatcatatgc

SEQ ID NO: 90 - DNA, 300 bp, Artificial Sequence
  Synthetic construct
aaacaaacactgggtttaggcattctgctctcccagcaccgcatggctgagggtggaaaaaaataacatctgaaacaggccgggcttttgatgatacctccttatgacagacacatcgaaaaccaccgacggtgagtcacccacattctgtgcataccctctccgaggagcaggaagtgtggctattttaaaccctgaggcaatgagaagttttcagatgcgtcctaaggcgctccggccagcgccctgcatgcacacgagggccttcctcagtgtggccccagcacatctgtagacctg

SEQ ID NO: 91 - DNA, 300 bp, Artificial Sequence
  Synthetic construct
attttgactcacaatgttgaaaccagattataaatgagtcatcagtgaatcgaccacaaagagcctttgcggaggtgatttacaggagagctctgatgtctgctgtcccctgcacacgcttcacagagatgctgtcagacgcagagctggtctggggcatctgttgccgcgtcagctcaaaaggatgctgtgttgtcaccaatgggattccccagcccaggcggtgttgcggtcccacccacacaaggaaggcggccatcactgaataatgcttgtggttacatcatcattgctggtttc

SEQ ID NO: 92 - DNA, 300 bp, Artificial Sequence
  Synthetic construct
gggatttcctctgctttttcaactaaaatcagctctttcccaaaagcctgtgctgcctgttgtgttttctctgtgtgtgttttgaaatggccttgcgcaccctccagactctctgcctccggggcaagtctgccttttccctgtttccactttgcatactgcataacttccttctctgccccacatggacacacgccctcttattcatgcatccgcggctcttgctgcattcgctcggcagcaaagccacaggctcccttgtggatgtcccttgtggagatttgtacttttttaccccac

SEQ ID NO: 93 - DNA, 300 bp, Artificial Sequence
  Synthetic construct
gagcacagaagacgacccagctgaggctggcaggagagacgaaggccccgccagatcccggaagccgcgcccttctgtccggctgcacgcccgattggacggttcctacgtcagcgcccctgattggatagggctccaggccccgccccctcagtccctgagtgacggaggatgtgatcggacgctgggctgagggcgacaaagtgacaggttcttggctgcagccttttcatgcagggcttcctgcttgcgctgggcctggcccagcccagggggcattttcatttaaccttttgtata

SEQ ID NO: 94 - DNA, 300 bp, Artificial Sequence
  Synthetic construct
agtttggatgttctctgtggagagggaataaaaccattgcctgttccctggagggaattggatgctgaagcttctacctttaacaggggcatgggtgcagttccagcctctgccagcaggctgggccctgtgcccacttttgaaagaccttcagggctgtggggcatgagatgagagagggagggaagataatctggctcactgccgggcactttatgtgacttacctccttaattcccccgggcacagccctgagaggaggttggcagtgtctgcattttacagatggggaacttgagg

SEQ ID NO: 95 - DNA, 300 bp, Artificial Sequence
  Synthetic construct
aaacttcgtctcaaaaacaaaacaaaacaaagcgaaaaaacaaaaaaagtttcattgtttcacctccacacagctctgtctgcattttgagcaatggccaccagagggcaggaagaaccaatctataaagcacacaagggtttcaccaactttgaagtcctccgttagaaggcaagttgtccactaatatgtaggaacgattaatggccaccagagggcaggaagaaccaatctataaagcgcacaagggtttcaccaactttgaagtcctccgttagaaggcaagttgtccactaatat

SEQ ID NO: 96 - DNA, 300 bp, Artificial Sequence
  Synthetic construct
gaagcagccagaagacctggttctcccaagcctgctacttgctggccatgtaaccttgagcaagttatttcctcctctgcaaaaggaagacaataccctcctgcctacttcactcagacgttctgaagatcgatgtagcaatgtggtgtagacatgcttttgtaacgtggacacacccagacaggaataagtcttgtccagggaatattttttgacaaacactgcttaactggtttgtcctctgagtgtcacaacttttggcagaacttggtagttggaggtcagtggttggctggttca

SEQ ID NO: 97 - DNA, 300 bp, Artificial Sequence
  Synthetic construct
cagcccttcctcacctcatcactccccatccccccaagatatagaaaggccgtgacagctgccagccctgcacatgctcttgtttcaacagcggcgattgcacatcacgtagtccccacgtgacctgtcgggcctagggcaagcgcaaagctttcggaaacccgaattattgcaaccttgacttcctgcctgtctctgaggctcccgggctgtgctttaagctggacaggcacctgctttacagggaaaaggaccaaggtccggagaggaaaggggcttgtcccaggatacgcagcaagt

SEQ ID NO: 98 - DNA, 300 bp, Artificial Sequence
  Synthetic construct
ctaggagctctgtgcggaaccgcgtccagccgccgactcactgacacatcacaatgagtcacgtgctctgtgcaccgggcggatttgtcagatccgctgctgcatcacggctcggcagggctctctgggttctcagtgccctcctaggtctgcaatgcagtgcgggagaggaggaatatgggcttgtgggggcaggggcagcgcccggactcctcccggggcaggactcccagaaacgcaggaagcgatgacgctgctcagataaaccctggcgctctgcgctggcgtcctggtcaggag

SEQ ID NO: 99 - DNA, 300 bp, Artificial Sequence
  Synthetic construct
catgtgagctcaattaatacaacatatggttactgtacgcccaaaggcaacgcattcaaattgctttgtaccatgtaaaacacacactcttgaaaaacagacgcctagtgcggaatcctgtgcacgcctttaactcctccaaacgagcagggggcgtcatggattagcatgtcccggggttcgggaatcagcatttccgaggaaaggggcgctcaggagatatccccacccccgatgaggggcactgtcgtggatgagtttaaaccacgccataggcagccaagaactgagctcccgatg

SEQ ID NO: 100 - DNA, 300 bp, Artificial Sequence
  Synthetic construct
gggaccaatccagaagcagcacccagaccggtttacccggttccaggaccttgggcgaagtccacccgcccgagggcagggacgacgcaggccacgccgcggcccagttgctagccaggcagggtggggatttgatcttgccaaggaaatgtgagcgggaggccgagcgttggaggtgggtaagtcgtcactatgcagggcggagccatcctgtgtctatcacgcccaagggcggtgcatgcaaattgactcccgcatttggcttttccccgggctccgtctccgcgcgctgcaacccgc

SEQ ID NO: 101 - DNA, 20 bp, Artificial Sequence
  Synthetic construct
ggggctctcaaggactctac

SEQ ID NO: 102 - DNA, 20 bp, Artificial Sequence
  Synthetic construct
cgagggcagaaaggagagac

SEQ ID NO: 103 - DNA, 18 bp, Artificial Sequence
  Synthetic construct
gtttccgtacgcagcctg

SEQ ID NO: 104 - DNA, 20 bp, Artificial Sequence
  Synthetic construct
caatgagcaggaagatgggc

SEQ ID NO: 105 - DNA, 20 bp, Artificial Sequence
  Synthetic construct
aaggctcagtgtgtgtatgc

SEQ ID NO: 106 - DNA, 20 bp, Artificial Sequence
  Synthetic construct
ggttactgactgctccccat

SEQ ID NO: 107 - DNA, 20 bp, Artificial Sequence
  Synthetic construct
taaccttcccttggcttcca

SEQ ID NO: 108 - DNA, 20 bp, Artificial Sequence
  Synthetic construct
gagagaaggaaagggagggg

SEQ ID NO: 109 - DNA, 20 bp, Artificial Sequence
  Synthetic construct
acctctagaccaagtgcctg

SEQ ID NO: 110 - DNA, 20 bp, Artificial Sequence
  Synthetic construct
ggtggcttcagagatggagt

SEQ ID NO: 111 - DNA, 20 bp, Artificial Sequence
  Synthetic construct
ctgacggcaaattcctccag

SEQ ID NO: 112 - DNA, 20 bp, Artificial Sequence
  Synthetic construct
gcttgtctgtcatctgaggc

SEQ ID NO: 113 - DNA, 18 bp, Artificial Sequence
  Synthetic construct
gaccagccaatctcccgg

SEQ ID NO: 114 - DNA, 19 bp, Artificial Sequence
  Synthetic construct
gagatccattggttgcggc

SEQ ID NO: 115 - DNA, 20 bp, Artificial Sequence
  Synthetic construct
gtgtgagccaagtgttgacc

SEQ ID NO: 116 - DNA, 20 bp, Artificial Sequence
  Synthetic construct
ttcatcctgcctgcctagag

SEQ ID NO: 117 - DNA, 20 bp, Artificial Sequence
  Synthetic construct
ctttccactcacaccttgcc

SEQ ID NO: 118 - DNA, 20 bp, Artificial Sequence
  Synthetic construct
aggcacaaaagaggcaaagg

SEQ ID NO: 119 - DNA, 20 bp, Artificial Sequence
  Synthetic construct
ggatgagtcactggatccgt

SEQ ID NO: 120 - DNA, 20 bp, Artificial Sequence
  Synthetic construct
gcctctgtcccttctccatt

SEQ ID NO: 121 - DNA, 20 bp, Artificial Sequence
  Synthetic construct
aatccagtcccagttcccag

SEQ ID NO: 122 - DNA, 20 bp, Artificial Sequence
  Synthetic construct
actggcctctcaacacctac

SEQ ID NO: 123 - DNA, 20 bp, Artificial Sequence
  Synthetic construct
cccagagctgcaatgtgtac

SEQ ID NO: 124 - DNA, 20 bp, Artificial Sequence
  Synthetic construct
tacggatgaggaggctggta

SEQ ID NO: 125 - DNA, 20 bp, Artificial Sequence
  Synthetic construct
cctgtacctctgcagtgcta

SEQ ID NO: 126 - DNA, 20 bp, Artificial Sequence
  Synthetic construct
tcctgggcagagtgttttca

SEQ ID NO: 127 - DNA, 20 bp, Artificial Sequence
  Synthetic construct
cctccttcccatttctgcct

SEQ ID NO: 128 - DNA, 20 bp, Artificial Sequence
  Synthetic construct
ccttttccatttccagcccc

SEQ ID NO: 129 - DNA, 20 bp, Artificial Sequence
  Synthetic construct
gaatccgcagatcctcaagc

SEQ ID NO: 130 - DNA, 20 bp, Artificial Sequence
  Synthetic construct
aagttcttcttcccgccctc

SEQ ID NO: 131 - DNA, 20 bp, Artificial Sequence
  Synthetic construct
accattcagtgtaagcccca

SEQ ID NO: 132 - DNA, 20 bp, Artificial Sequence
  Synthetic construct
tctttccaccatgcacgttg

SEQ ID NO: 133 - DNA, 20 bp, Artificial Sequence
  Synthetic construct
acaccaccacctccttcttc

SEQ ID NO: 134 - DNA, 21 bp, Artificial Sequence
  Synthetic construct
ggaaagtgcaaatgaacccca

SEQ ID NO: 135 - DNA, 20 bp, Artificial Sequence
  Synthetic construct
tggttccctcctcacatcag

SEQ ID NO: 136 - DNA, 20 bp, Artificial Sequence
  Synthetic construct
ttgcaacctccgcttgaaaa

SEQ ID NO: 137 - DNA, 20 bp, Artificial Sequence
  Synthetic construct
ttgggaaggcaagaggatga

SEQ ID NO: 138 - DNA, 20 bp, Artificial Sequence
  Synthetic construct
gtgttcaagccctccctcta

SEQ ID NO: 139 - DNA, 20 bp, Artificial Sequence
  Synthetic construct
ctcacaccgtctcactaggg

SEQ ID NO: 140 - DNA, 19 bp, Artificial Sequence
  Synthetic construct
gaattccacagacaccgcg

SEQ ID NO: 141 - DNA, 20 bp, Artificial Sequence
  Synthetic construct
cctcgtagggcttgaaatgt

SEQ ID NO: 142 - DNA, 23 bp, Artificial Sequence
  Synthetic construct
agaagaatttggcgaggattacc

SEQ ID NO: 143 - DNA, 20 bp, Artificial Sequence
  Synthetic construct
ttgagcgcagtctggaaatg

SEQ ID NO: 144 - DNA, 20 bp, Artificial Sequence
  Synthetic construct
gagatgagccctgtcacagt

SEQ ID NO: 145 - DNA, 20 bp, Artificial Sequence
  Synthetic construct
aagtcctagagcaccggaac

SEQ ID NO: 146 - DNA, 20 bp, Artificial Sequence
  Synthetic construct
cttttctccgcagcgatacc

SEQ ID NO: 147 - DNA, 20 bp, Artificial Sequence
  Synthetic construct
ggaaaatcccttgaagccgg

SEQ ID NO: 148 - DNA, 20 bp, Artificial Sequence
  Synthetic construct
tgggctgttgtaatggtgtg

SEQ ID NO: 149 - DNA, 20 bp, Artificial Sequence
  Synthetic construct
tgatgagctactacgcctgg

SEQ ID NO: 150 - DNA, 20 bp, Artificial Sequence
  Synthetic construct
tgggaagttgagggatgtgt

SEQ ID NO: 151 - DNA, 20 bp, Artificial Sequence
  Synthetic construct
cggggaggagatgagctaaa

SEQ ID NO: 152 - DNA, 20 bp, Artificial Sequence
  Synthetic construct
agtacgtcgaacaggggatc

SEQ ID NO: 153 - DNA, 20 bp, Artificial Sequence
  Synthetic construct
tggattgatacggggctctt

SEQ ID NO: 154 - DNA, 20 bp, Artificial Sequence
  Synthetic construct
cgacagctggtttcacaagt

SEQ ID NO: 155 - DNA, 18 bp, Artificial Sequence
  Synthetic construct
ctgagaggcccgcaatgt

SEQ ID NO: 156 - DNA, 18 bp, Artificial Sequence
  Synthetic construct
cactcagatctcgccgcg

SEQ ID NO: 157 - DNA, 20 bp, Artificial Sequence
  Synthetic construct
gagcggtgcaaaggttctta

SEQ ID NO: 158 - DNA, 20 bp, Artificial Sequence
  Synthetic construct
cctacctcgtgctcttggaa

SEQ ID NO: 159 - DNA, 19 bp, Artificial Sequence
  Synthetic construct
ctctccaagcaccactccc

SEQ ID NO: 160 - DNA, 20 bp, Artificial Sequence
  Synthetic construct
tgtggagactgaaccttgca

SEQ ID NO: 161 - DNA, 20 bp, Artificial Sequence
  Synthetic construct
cagtccctacaccccacaat

SEQ ID NO: 162 - DNA, 20 bp, Artificial Sequence
  Synthetic construct
catttcccagaagaccacgc

SEQ ID NO: 163 - DNA, 20 bp, Artificial Sequence
  Synthetic construct
tggttaaggtgtaggggtgg

SEQ ID NO: 164 - DNA, 20 bp, Artificial Sequence
  Synthetic construct
agcacagagttaccacctcc

SEQ ID NO: 165 - DNA, 20 bp, Artificial Sequence
  Synthetic construct
gtgtaaacaaacccaggccc

SEQ ID NO: 166 - DNA, 19 bp, Artificial Sequence
  Synthetic construct
gactccatcaccgtcccaa

SEQ ID NO: 167 - DNA, 20 bp, Artificial Sequence
  Synthetic construct
gcgcgcttttaaggagagtt

SEQ ID NO: 168 - DNA, 20 bp, Artificial Sequence
  Synthetic construct
ggctctgacggcattcattc

SEQ ID NO: 169 - DNA, 20 bp, Artificial Sequence
  Synthetic construct
gatgcagatgtcaaaacgcg

SEQ ID NO: 170 - DNA, 19 bp, Artificial Sequence
  Synthetic construct
gagggtctcgcttgtttgc

SEQ ID NO: 171 - DNA, 20 bp, Artificial Sequence
  Synthetic construct
aggagttcagcatagcacga

SEQ ID NO: 172 - DNA, 20 bp, Artificial Sequence
  Synthetic construct
caagctgtgccataacccaa

SEQ ID NO: 173 - DNA, 19 bp, Artificial Sequence
  Synthetic construct
ctgtcctcctccctccctc

SEQ ID NO: 174 - DNA, 20 bp, Artificial Sequence
  Synthetic construct
tttcttgtccacactcctgg

SEQ ID NO: 175 - DNA, 20 bp, Artificial Sequence
  Synthetic construct
acacccgacagagtccaatg

SEQ ID NO: 176 - DNA, 20 bp, Artificial Sequence
  Synthetic construct
ggtagtcactcctttggcct

SEQ ID NO: 177 - DNA, 20 bp, Artificial Sequence
  Synthetic construct
cggctctgagtctgaagcta

SEQ ID NO: 178 - DNA, 20 bp, Artificial Sequence
  Synthetic construct
ggagcctctgtacctgtgac

SEQ ID NO: 179 - DNA, 18 bp, Artificial Sequence
  Synthetic construct
ccccacagtggtcacgag

SEQ ID NO: 180 - DNA, 20 bp, Artificial Sequence
  Synthetic construct
ggggaaggagaacagagagg

SEQ ID NO: 181 - DNA, 20 bp, Artificial Sequence
  Synthetic construct
gctcaaatgatccgctctcc

SEQ ID NO: 182 - DNA, 20 bp, Artificial Sequence
  Synthetic construct
gactctgatgctgccatgtg

SEQ ID NO: 183 - DNA, 20 bp, Artificial Sequence
  Synthetic construct
gcggaagtcatgtctggaac

SEQ ID NO: 184 - DNA, 20 bp, Artificial Sequence
  Synthetic construct
cgaggccaggaaacactagt

SEQ ID NO: 185 - DNA, 20 bp, Artificial Sequence
  Synthetic construct
ggcacaaaggaaacgaagga

SEQ ID NO: 186 - DNA, 20 bp, Artificial Sequence
  Synthetic construct
ccaagcacctcaagccaaat

SEQ ID NO: 187 - DNA, 20 bp, Artificial Sequence
  Synthetic construct
cctccctggtagaatctggc

SEQ ID NO: 188 - DNA, 20 bp, Artificial Sequence
  Synthetic construct
gtgtctcaggctcagttcct

SEQ ID NO: 189 - DNA, 18 bp, Artificial Sequence
  Synthetic construct
ctgtgctgttccgtgtgc

SEQ ID NO: 190 - DNA, 20 bp, Artificial Sequence
  Synthetic construct
gagaccttttcacgggatct

SEQ ID NO: 191 - DNA, 20 bp, Artificial Sequence
  Synthetic construct
acctctgtgtgaagtgctct

SEQ ID NO: 192 - DNA, 20 bp, Artificial Sequence
  Synthetic construct
gcgagtcaaaggtgtggttt

SEQ ID NO: 193 - DNA, 20 bp, Artificial Sequence
  Synthetic construct
tctaagactggctgctctgc

SEQ ID NO: 194 - DNA, 20 bp, Artificial Sequence
  Synthetic construct
aaaacgccctttctgctcag

SEQ ID NO: 195 - DNA, 20 bp, Artificial Sequence
  Synthetic construct
gggtgggtctacttctgagg

SEQ ID NO: 196 - DNA, 20 bp, Artificial Sequence
  Synthetic construct
ctccctttcggcttcatgtg

SEQ ID NO: 197 - DNA, 20 bp, Artificial Sequence
  Synthetic construct
tggaatggaatcaacccgag

SEQ ID NO: 198 - DNA, 20 bp, Artificial Sequence
  Synthetic construct
cgaaaccgttccattccagt

SEQ ID NO: 199 - DNA, 20 bp, Artificial Sequence
  Synthetic construct
aacaggggagttctcatgcc

SEQ ID NO: 200 - DNA, 20 bp, Artificial Sequence
  Synthetic construct
agccacatcagagacagagc

SEQ ID NO: 201 - DNA, 20 bp, Artificial Sequence
  Synthetic construct
gttcaaatgtcaggcctgct

SEQ ID NO: 202 - DNA, 20 bp, Artificial Sequence
  Synthetic construct
cacctccaaagacaaacgca

SEQ ID NO: 203 - DNA, 20 bp, Artificial Sequence
  Synthetic construct
gcccacgtgactagcatagg

SEQ ID NO: 204 - DNA, 19 bp, Artificial Sequence
  Synthetic construct
gagcgagaactgggagtgc

SEQ ID NO: 205 - DNA, 20 bp, Artificial Sequence
  Synthetic construct
atcaccctgagccttggaag

SEQ ID NO: 206 - DNA, 20 bp, Artificial Sequence
  Synthetic construct
caggtaatgcagcggttcat

SEQ ID NO: 207 - DNA, 20 bp, Artificial Sequence
  Synthetic construct
caagggacccagagatcaca

SEQ ID NO: 208 - DNA, 20 bp, Artificial Sequence
  Synthetic construct
acagcaaacacaaaagccca

SEQ ID NO: 209 - DNA, 20 bp, Artificial Sequence
  Synthetic construct
tctgcgaaagaggaggtgac

SEQ ID NO: 210 - DNA, 20 bp, Artificial Sequence
  Synthetic construct
cattccagaaccacaggctg

SEQ ID NO: 211 - DNA, 19 bp, Artificial Sequence
  Synthetic construct
agcctccgtcagtgtcttc

SEQ ID NO: 212 - DNA, 20 bp, Artificial Sequence
  Synthetic construct
tgagactctagcccttccct

SEQ ID NO: 213 - DNA, 20 bp, Artificial Sequence
  Synthetic construct
ctctccataagacacgccca

SEQ ID NO: 214 - DNA, 20 bp, Artificial Sequence
  Synthetic construct
aagaggcgggtcattcagaa

SEQ ID NO: 215 - DNA, 20 bp, Artificial Sequence
  Synthetic construct
caaagtgccgggattacagg

SEQ ID NO: 216 - DNA, 20 bp, Artificial Sequence
  Synthetic construct
tcccaaagagtgtcacagca

SEQ ID NO: 217 - DNA, 20 bp, Artificial Sequence
  Synthetic construct
ccctctcctcttgcatgact

SEQ ID NO: 218 - DNA, 20 bp, Artificial Sequence
  Synthetic construct
atgcctctttgctgttctgc

SEQ ID NO: 219 - DNA, 20 bp, Artificial Sequence
  Synthetic construct
tcgccactctcagtcaaact

SEQ ID NO: 220 - DNA, 21 bp, Artificial Sequence
  Synthetic construct
ccctagggcaaaatcaactgt

SEQ ID NO: 221 - DNA, 20 bp, Artificial Sequence
  Synthetic construct
tgttgtagcctgagtcggtt

SEQ ID NO: 222 - DNA, 20 bp, Artificial Sequence
  Synthetic construct
ccccacttgcttctgtagga

SEQ ID NO: 223 - DNA, 20 bp, Artificial Sequence
  Synthetic construct
gggaaggaaggttggtgaga

SEQ ID NO: 224 - DNA, 20 bp, Artificial Sequence
  Synthetic construct
accgtatagagcagagtggc

SEQ ID NO: 225 - DNA, 20 bp, Artificial Sequence
  Synthetic construct
tccaagtctaagggtgctgg

SEQ ID NO: 226 - DNA, 20 bp, Artificial Sequence
  Synthetic construct
taccatcctctgctttgcca

SEQ ID NO: 227 - DNA, 20 bp, Artificial Sequence
  Synthetic construct
gagggctttcagaactcagc

SEQ ID NO: 228 - DNA, 20 bp, Artificial Sequence
  Synthetic construct
gcaacgccttcctgttaaga

SEQ ID NO: 229 - DNA, 20 bp, Artificial Sequence
  Synthetic construct
ttccctcgacctcccttcta

SEQ ID NO: 230 - DNA, 20 bp, Artificial Sequence
  Synthetic construct
ccctgtcctgccagctatag

SEQ ID NO: 231 - DNA, 20 bp, Artificial Sequence
  Synthetic construct
gactcctgaggaaaccagct

SEQ ID NO: 232 - DNA, 20 bp, Artificial Sequence
  Synthetic construct
cagagtggaaggttaacgcg

SEQ ID NO: 233 - DNA, 20 bp, Artificial Sequence
  Synthetic construct
gcaagagagactgagagcac

SEQ ID NO: 234 - DNA, 20 bp, Artificial Sequence
  Synthetic construct
ccctcctccctcattcactc

SEQ ID NO: 235 - DNA, 20 bp, Artificial Sequence
  Synthetic construct
gctgcagctgctgtattcac

SEQ ID NO: 236 - DNA, 20 bp, Artificial Sequence
  Synthetic construct
aggggtacagggcagaaaat

SEQ ID NO: 237 - DNA, 20 bp, Artificial Sequence
  Synthetic construct
ggaacaaggaggagcagaca

SEQ ID NO: 238 - DNA, 20 bp, Artificial Sequence
  Synthetic construct
ctccatgagtcaggctgaga

SEQ ID NO: 239 - DNA, 20 bp, Artificial Sequence
  Synthetic construct
ggccaatcaacactgtgact

SEQ ID NO: 240 - DNA, 19 bp, Artificial Sequence
  Synthetic construct
tccagtgctccgggatttc

SEQ ID NO: 241 - DNA, 18 bp, Artificial Sequence
  Synthetic construct
ccgaactggcgctcaaca

SEQ ID NO: 242 - DNA, 20 bp, Artificial Sequence
  Synthetic construct
ctctgcacttattggtcggg

SEQ ID NO: 243 - DNA, 19 bp, Artificial Sequence
  Synthetic construct
tgtgtgcattcatctcgca

SEQ ID NO: 244 - DNA, 20 bp, Artificial Sequence
  Synthetic construct
cctcaaagcgctccaaatgt

SEQ ID NO: 245 - DNA, 20 bp, Artificial Sequence
  Synthetic construct
gcaggagttaaagtacccgc

SEQ ID NO: 246 - DNA, 20 bp, Artificial Sequence
  Synthetic construct
gctgtgctcataggctctcc

SEQ ID NO: 247 - DNA, 20 bp, Artificial Sequence
  Synthetic construct
cttcctcttcctcaggctcc

SEQ ID NO: 248 - DNA, 19 bp, Artificial Sequence
  Synthetic construct
cggtgactcagagctttgc

SEQ ID NO: 249 - DNA, 20 bp, Artificial Sequence
  Synthetic construct
ggctggggaggttcttctag

SEQ ID NO: 250 - DNA, 20 bp, Artificial Sequence
  Synthetic construct
ttcatgtccacctcctcagc

SEQ ID NO: 251 - DNA, 20 bp, Artificial Sequence
  Synthetic construct
ccgcagcttcctatcctgta

SEQ ID NO: 252 - DNA, 20 bp, Artificial Sequence
  Synthetic construct
accaggcttctcatcttcct

SEQ ID NO: 253 - DNA, 20 bp, Artificial Sequence
  Synthetic construct
atggcagcacagagagaagt

SEQ ID NO: 254 - DNA, 20 bp, Artificial Sequence
  Synthetic construct
tggctcagctctctctcatg

SEQ ID NO: 255 - DNA, 20 bp, Artificial Sequence
  Synthetic construct
agctgactgggacctgaaag

SEQ ID NO: 256 - DNA, 18 bp, Artificial Sequence
  Synthetic construct
cccgagccagccaatcag

SEQ ID NO: 257 - DNA, 20 bp, Artificial Sequence
  Synthetic construct
caagagtggaaaacctgccc

SEQ ID NO: 258 - DNA, 20 bp, Artificial Sequence
  Synthetic construct
gaggggaagatggctcactg

SEQ ID NO: 259 - DNA, 20 bp, Artificial Sequence
  Synthetic construct
ctcttcccctccctcacttg

SEQ ID NO: 260 - DNA, 20 bp, Artificial Sequence
  Synthetic construct
catgggctcacatcctccta

SEQ ID NO: 261 - DNA, 20 bp, Artificial Sequence
  Synthetic construct
agccacacacttatctgcct

SEQ ID NO: 262 - DNA, 20 bp, Artificial Sequence
  Synthetic construct
cccgagctacactagatgca

SEQ ID NO: 263 - DNA, 20 bp, Artificial Sequence
  Synthetic construct
aagtgggcagggcttaaaac

SEQ ID NO: 264 - DNA, 20 bp, Artificial Sequence
  Synthetic construct
gggctccactccattctgaa

SEQ ID NO: 265 - DNA, 20 bp, Artificial Sequence
  Synthetic construct
aagaggaggatggagcagag

SEQ ID NO: 266 - DNA, 20 bp, Artificial Sequence
  Synthetic construct
gagagagggaagcgagacag

SEQ ID NO: 267 - DNA, 20 bp, Artificial Sequence
  Synthetic construct
ggtggggaggaagttctgaa

SEQ ID NO: 268 - DNA, 20 bp, Artificial Sequence
  Synthetic construct
ctttgcaaccctactgtgcc

SEQ ID NO: 269 - DNA, 20 bp, Artificial Sequence
  Synthetic construct
atgacgaaactggtgcatgt

SEQ ID NO: 270 - DNA, 20 bp, Artificial Sequence
  Synthetic construct
tcaagaatgcagactccgga

SEQ ID NO: 271 - DNA, 20 bp, Artificial Sequence
  Synthetic construct
ccctccactggacactgaat

SEQ ID NO: 272 - DNA, 20 bp, Artificial Sequence
  Synthetic construct
agaagccaaccaagaaaccg

SEQ ID NO: 273 - DNA, 19 bp, Artificial Sequence
  Synthetic construct
ttggaaagaagggtggcca

SEQ ID NO: 274 - DNA, 20 bp, Artificial Sequence
  Synthetic construct
ctcatttcaccccgcctcta

SEQ ID NO: 275 - DNA, 20 bp, Artificial Sequence
  Synthetic construct
tttgaggttgggaaagcagc

SEQ ID NO: 276 - DNA, 20 bp, Artificial Sequence
  Synthetic construct
agcagatttccccttagcga

SEQ ID NO: 277 - DNA, 20 bp, Artificial Sequence
  Synthetic construct
tgcatctgaccttggtctgt

SEQ ID NO: 278 - DNA, 20 bp, Artificial Sequence
  Synthetic construct
ggccatgagaagtcctgagt

SEQ ID NO: 279 - DNA, 20 bp, Artificial Sequence
  Synthetic construct
agacacatcgaaaaccaccg

SEQ ID NO: 280 - DNA, 20 bp, Artificial Sequence
  Synthetic construct
gccttaggacgcatctgaaa

SEQ ID NO: 281 - DNA, 20 bp, Artificial Sequence
  Synthetic construct
aggagagctctgatgtctgc

SEQ ID NO: 282 - DNA, 20 bp, Artificial Sequence
  Synthetic construct
gcatccttttgagctgacgc

SEQ ID NO: 283 - DNA, 20 bp, Artificial Sequence
  Synthetic construct
tgtgctgcctgttgtgtttt

SEQ ID NO: 284 - DNA, 20 bp, Artificial Sequence
  Synthetic construct
atgtggggcagagaaggaag

SEQ ID NO: 285 - DNA, 18 bp, Artificial Sequence
  Synthetic construct
caggagagacgaaggccc

SEQ ID NO: 286 - DNA, 20 bp, Artificial Sequence
  Synthetic construct
tcacatcctccgtcactcag

SEQ ID NO: 287 - DNA, 20 bp, Artificial Sequence
  Synthetic construct
ctttaacaggggcatgggtg

SEQ ID NO: 288 - DNA, 20 bp, Artificial Sequence
  Synthetic construct
tctctcatctcatgccccac

SEQ ID NO: 289 - DNA, 21 bp, Artificial Sequence
  Synthetic construct
cagctctgtctgcattttgag

SEQ ID NO: 290 - DNA, 20 bp, Artificial Sequence
  Synthetic construct
tggtggccattaatcgttcc

SEQ ID NO: 291 - DNA, 20 bp, Artificial Sequence
  Synthetic construct
ctggccatgtaaccttgagc

SEQ ID NO: 292 - DNA, 21 bp, Artificial Sequence
  Synthetic construct
tgtgtccacgttacaaaagca

SEQ ID NO: 293 - DNA, 20 bp, Artificial Sequence
  Synthetic construct
atagaaaggccgtgacagct

SEQ ID NO: 294 - DNA, 20 bp, Artificial Sequence
  Synthetic construct
gcaggaagtcaaggttgcaa

SEQ ID NO: 295 - DNA, 20 bp, Artificial Sequence
  Synthetic construct
gggagaggaggaatatgggc

SEQ ID NO: 296 - DNA, 20 bp, Artificial Sequence
  Synthetic construct
agggtttatctgagcagcgt

SEQ ID NO: 297 - DNA, 20 bp, Artificial Sequence
  Synthetic construct
gggcgtcatggattagcatg

SEQ ID NO: 298 - DNA, 20 bp, Artificial Sequence
  Synthetic construct
cagttcttggctgcctatgg

SEQ ID NO: 299 - DNA, 20 bp, Artificial Sequence
  Synthetic construct
gaccaatccagaagcagcac

SEQ ID NO: 300 - DNA, 20 bp, Artificial Sequence
  Synthetic construct
gcaagatcaaatccccaccc

SEQ ID NO: 301 - DNA, 8 bp, Artificial Sequence
  Synthetic construct
taaggcga

SEQ ID NO: 302 - DNA, 8 bp, Artificial Sequence
  Synthetic construct
cgtactag

SEQ ID NO: 303 - DNA, 8 bp, Artificial Sequence
  Synthetic construct
aggcagaa

SEQ ID NO: 304 - DNA, 8 bp, Artificial Sequence
  Synthetic construct
tcctgagc

SEQ ID NO: 305 - DNA, 8 bp, Artificial Sequence
  Synthetic construct
ggactcct

SEQ ID NO: 306 - DNA, 8 bp, Artificial Sequence
  Synthetic construct
taggcatg

SEQ ID NO: 307 - DNA, 8 bp, Artificial Sequence
  Synthetic construct
ctctctac

SEQ ID NO: 308 - DNA, 8 bp, Artificial Sequence
  Synthetic construct
cagagagg

SEQ ID NO: 309 - DNA, 8 bp, Artificial Sequence
  Synthetic construct
gctacgct

SEQ ID NO: 310 - DNA, 8 bp, Artificial Sequence
  Synthetic construct
cgaggctg

SEQ ID NO: 311 - DNA, 8 bp, Artificial Sequence
  Synthetic construct
aagaggca

SEQ ID NO: 312 - DNA, 8 bp, Artificial Sequence
  Synthetic construct
gtagagga

SEQ ID NO: 313 - DNA, 8 bp, Artificial Sequence
  Synthetic construct
gtcgtgat

SEQ ID NO: 314 - DNA, 8 bp, Artificial Sequence
  Synthetic construct
accactgt

SEQ ID NO: 315 - DNA, 8 bp, Artificial Sequence
  Synthetic construct
tggatctg

SEQ ID NO: 316 - DNA, 8 bp, Artificial Sequence
  Synthetic construct
ccgtttgt

SEQ ID NO: 317 - DNA, 8 bp, Artificial Sequence
  Synthetic construct
tgctgggt

SEQ ID NO: 318 - DNA, 8 bp, Artificial Sequence
  Synthetic construct
gaggggtt

SEQ ID NO: 319 - DNA, 8 bp, Artificial Sequence
  Synthetic construct
aggttggg

SEQ ID NO: 320 - DNA, 8 bp, Artificial Sequence
  Synthetic construct
gtgtggtg

SEQ ID NO: 321 - DNA, 8 bp, Artificial Sequence
  Synthetic construct
tgggtttc

SEQ ID NO: 322 - DNA, 8 bp, Artificial Sequence
  Synthetic construct
tggtcaca

SEQ ID NO: 323 - DNA, 8 bp, Artificial Sequence
  Synthetic construct
ttgaccct

SEQ ID NO: 324 - DNA, 8 bp, Artificial Sequence
  Synthetic construct
ccactcct